SEQUENCE LISTING (9 sequences)

SEQ ID NO: 1 - DNA, 80 bp, synthetic construct
  source 1..80: mol_type = other DNA
aaaaaaaaaaaaaaaaaaacatcacctacttccacctctcgtcggcagcgtcagatgtgtataagagacagcgaccgtaa

SEQ ID NO: 2 - DNA, 52 bp, synthetic construct
  source 1..52: mol_type = other DNA
aaaaaaaaaaaaaaaaaaccttcacattacctccacctctactcggatcttt

SEQ ID NO: 3 - DNA, 52 bp, synthetic construct
  source 1..52: mol_type = other DNA
aaaaaaaaaaaaaaaaaatctctacttccaacttacctccacccgggaatag

SEQ ID NO: 4 - DNA, 137 bp, synthetic construct
  source 1..137: mol_type = other DNA
  modified_base 1: mod_base = OTHER; note = 5'-Phosphate modified nucleotide
  misc_difference 114: note = a, c, t, g, unknown or other
  misc_difference 115: note = a, c, t, g, unknown or other
  misc_difference 116: note = a, c, t, g, unknown or other
  misc_difference 117: note = a, c, t, g, unknown or other
  misc_difference 118: note = a, c, t, g, unknown or other
  misc_difference 119: note = a, c, t, g, unknown or other
  misc_difference 120: note = a, c, t, g, unknown or other
ctgtctcttatacacatctgacgctgccgacgagaggtggaagtaggtgatgtgatatcgagggtgaaggtgaataggggtctcgtgggctcggagatgtgtataagagacagnnnnnnngcacagttaaagatccg

SEQ ID NO: 5 - DNA, 54 bp, synthetic construct
  source 1..54: mol_type = other DNA
  modified_base 1: mod_base = OTHER; note = 5'-Phosphate modified nucleotide
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
agtagaggtggaggtaatgtgaaggnnnnnnnnnnnnttgctcctctattcccg

SEQ ID NO: 6 - DNA, 54 bp, synthetic construct
  source 1..54: mol_type = other DNA
  modified_base 1: mod_base = OTHER; note = 5'-Phosphate modified nucleotide
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
ggtggaggtaagttggaagtagagannnnnnnnnnnnaggtcaagttacggtcg

SEQ ID NO: 7 - DNA, 134 bp, synthetic construct
  source 1..134: mol_type = other DNA
  modified_base 1: mod_base = OTHER; note = 5'-Phosphate modified nucleotide
  misc_difference 114: note = a, c, t, g, unknown or other
  misc_difference 115: note = a, c, t, g, unknown or other
  misc_difference 116: note = a, c, t, g, unknown or other
  misc_difference 117: note = a, c, t, g, unknown or other
  misc_difference 118: note = a, c, t, g, unknown or other
  misc_difference 119: note = a, c, t, g, unknown or other
  misc_difference 120: note = a, c, t, g, unknown or other
ctgtctcttatacacatctgacgctgccgacgagaggtggaagtaggtgatgtgatatcgagggtgaaggtgaataggggtctcgtgggctcggagatgtgtataagagacagnnnnnnngcacagttgatccg

SEQ ID NO: 8 - DNA, 51 bp, synthetic construct
  source 1..51: mol_type = other DNA
  modified_base 1: mod_base = OTHER; note = 5'-Phosphate modified nucleotide
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
agtagaggtggaggtaatgtgaaggnnnnnnnnnnnnttgctcctttcccg

SEQ ID NO: 9 - DNA, 68 bp, synthetic construct
  source 1..68: mol_type = other DNA
  modified_base 1: mod_base = OTHER; note = 5'-Phosphate modified nucleotide
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
ggtggaggtaagttggaagtagagannnnnnnnnnnnaggtcaagttacggtcgaggtcaagcggtcg

(19) United States
(12) Patent Application Publication
(10) Pub. No.: US 2026/0023072 A1
(43) Pub. Date: Jan. 22, 2026

(54) COMPOSITIONS AND METHODS FOR DETECTION OF PROTEIN ANALYTES

(71) Applicant: Range Biotechnologies, Inc., San Carlos, CA (US)

(72) Inventors: Brandon WILSON, Redwood City, CA (US); Nicolò MAGANZINI, Sunnyvale, CA (US)

(21) Appl. No.: 19/204,281

(22) Filed: May 9, 2025

Related U.S. Application Data
(63) Continuation of application No. PCT/US2023/080415, filed on Nov. 17, 2023.
(60) Provisional application No. 63/384,892, filed on Nov. 23, 2022.
(60) Provisional application No. 63/588,579, filed on Oct. 6, 2023.

(51) Int. Cl. G01N 33/53; G01N 33/68

(52) U.S. Cl. CPC G01N 33/5308; G01N 33/6803

(57) ABSTRACT

Described herein are methods and compositions for detecting an antigen using at least two antigen binders each comprising partially double-stranded nucleic acids wherein the partially double-stranded nucleic acids can template production of a linear or circular nucleic acid product.

### CROSS-REFERENCE

This application is a continuation of International Application No. PCT/US2023/080415, filed Nov. 17, 2023, which claims priority to U.S. Provisional Patent Application No. 63/384,892, filed Nov. 23, 2022 and U.S. Provisional Patent Application No. 63/588,579, filed Oct. 6, 2023, which applications are herein incorporated by reference in their entireties for all purposes.

### SEQUENCE LISTING

The instant application contains a Sequence Listing which has been submitted electronically in XML format and is hereby incorporated by reference in its entirety. Said XML copy, created on Aug. 20, 2025, is named 63490-701_301_SL.xml and is 37,206 bytes in size.

### SUMMARY

Described herein are methods, compositions, and systems for multiplex detection of protein analytes by sequencing (e.g., DNA sequencing). In some cases, such methods involve the use of coincidence-based detection of protein analytes involving at least two antigen binder-nucleic acid conjugates (e.g., two or more or three or more). Methods and compositions as described herein can allow for detection of protein analytes at higher multiplicity, with better dynamic range, improved throughput, or higher accuracy. In some cases, the methods, compositions, and systems described herein detect molecules of protein analyte down to single-molecule resolution.

Such methods can involve the use of multiple antigen-binders to template formation of a reporter structure (e.g. the annealed DNA structure shown in FIG. 1A) for which assembly is favored when its components are in high concentration. In the unbound-to-antigen state, antigen binders linked to the antigen binders are dilute, so that the reporter structure does not form by itself, as each component is in low local concentration. When the multiple antigen binders all bind to a target protein at the same time, this dramatically increases the effective concentrations of the reporter structure components locally around the target molecule, catalyzing the formation of the reporter structure (e.g. by base pair annealing). The reporter structure can then be locked into place using covalent linking (e.g. enzymatically by polymerase or ligase, or chemical cross linking). The reporter structure can then be detected. Alongside detection, any non-assembled reporter structure components can be eliminated (e.g., with exonuclease if the reporter structure is circular). Additionally, primers may be designed such that any non-assembled reporter structure is not exponentially amplified.

In some aspects, the present disclosure provides a method of detecting an antigen, comprising: (a) contacting an antigen with a plurality of antigen binders to form a complex comprising said antigen bound to antigen binders of said plurality of antigen binders, wherein said antigen binders are collectively capable of forming a circular nucleic acid product upon binding of said antigen binders to said antigen, wherein, during said contacting, said antigen binders comprise at least: (i) a first antigen binder comprising: (1) a first antigen-binding moiety capable of binding said antigen; and (2) first partially double-stranded nucleic acid linked to said first antigen-binding moiety; and (ii) a second antigen-binder comprising: (1) a second antigen-binding moiety capable of binding said antigen; and (2) a second partially double-stranded nucleic acid linked to said second antigen-binding moiety, wherein said first partially double-stranded nucleic acid and said second partially double-stranded nucleic acid are configured to template production of said circular nucleic acid product via at least said first partially double-stranded nucleic acid and said second double-stranded nucleic acid; (b) producing said circular nucleic acid product from at least said first partially double-stranded nucleic acid and said second-double-stranded nucleic acid. In some embodiments, the method further comprises incubating said complex with a ligase under conditions sufficient to produce said circular nucleic acid product via at least said first partially double-stranded nucleic acid and said second-double-stranded nucleic acid. In some embodiments, said first-partially double-stranded nucleic acid comprises: (i) a first proximal nucleic acid linked to said first antigen binder comprising a common hybridization region and an unhybridized overhanging 5′ end; and (ii) a first distal nucleic acid comprising a common hybridization region and an unhybridized overhanging 5′ end, wherein said first distal nucleic acid is hybridized to said first proximal nucleic acid via said hybridization region having said unhybridized overhanging 5′ end; and said second-partially double-stranded nucleic acid comprises: (i) said second proximal nucleic acid linked to said second antigen binder comprising a hybridization region and an unhybridized overhanging 5′ end; and (ii) said second distal nucleic acid comprising a common hybridization region an said unhybridized overhanging 5′ end, wherein said second distal nucleic acid is hybridized to said second proximal nucleic acid via said common hybridization region having said unhybridized overhanging 5′ end; wherein said free unhybridized 5′ end of said first proximal nucleic acid is configured to bind to said unhybridized overhanging 5′ end of said second distal nucleic acid, and said unhybridized overhanging 5′ end of said second proximal nucleic acid is configured to bind to said unhybridized overhanging 5′ end of said first distal nucleic acid; wherein said unhybridized overhanging 5′ end of said first proximal nucleic acid is configured to bind to said unhybridized overhanging 5′ end of said second distal nucleic acid, and said unhybridized overhanging 5′ end of said second proximal nucleic acid is configured to bind to said unhybridized overhanging 5′ end of said first distal nucleic acid. In some embodiments, said first-partially double-stranded nucleic acid comprises: (i) a first proximal nucleic acid linked to said first antigen binder comprising a common hybridization region and an unhybridized overhanging 3′ end; and (ii) a first distal nucleic acid comprising a common hybridization region and an unhybridized overhanging 3′ end, wherein said first distal nucleic acid is hybridized to said first proximal nucleic acid via said common hybridization region having said unhybridized overhanging 3′ end; and said second-partially double-stranded nucleic acid comprises: (i) a second proximal nucleic acid linked to said second antigen binder comprising a common hybridization region and an unhybridized overhanging 3′ end; and (ii) a second distal nucleic acid comprising a common hybridization region and an unhybridized overhanging 3′ end, wherein said second distal nucleic acid is hybridized to said second proximal nucleic acid via said common hybridization region having said unhybridized overhanging 3′ end; wherein said unhybridized overhanging 3′ end of said first proximal nucleic acid is configured to bind to said unhybridized overhanging 3′ end of said second distal nucleic acid, and said unhybridized overhanging 3′ end of said second proximal nucleic acid is configured to bind to said unhybridized overhanging 3′ end of said first distal nucleic acid. In some embodiments, the method further comprises (c) detecting said circular nucleic acid product comprising at least said first distal nucleic acid and said second distal nucleic acid, thereby detecting said antigen. In some embodiments, a length of said unhybridized 3′ or 5′ overhanging end of said first proximal nucleic acid and a length of said unhybridized 3′ or 5′ overhanging end of said second proximal nucleic acid are not equal in length. In some embodiments, the method further comprises contacting said antigen and said plurality of antigen binders with a tunable partially double-stranded nucleic acid, wherein said tunable partially double-stranded nucleic acid is configured to form said circular nucleic acid when said tunable partially double-stranded nucleic acid contacts at least said first partially double-stranded nucleic acid and said second partially double-stranded nucleic acid. In some embodiments, said plurality of antigen binders further comprises (iii) a third antigen binder comprising: (1) a third antigen-binding moiety capable of binding said antigen; and (2) a third partially double-stranded nucleic acid linked to the third antigen-binding moiety, wherein said third partially-double-stranded nucleic acid is configured to form said circular nucleic acid product when said first antigen binder, said second antigen binder, and said third antigen binder form a complex with said antigen; or (iv) a fourth antigen binder comprising (1) a fourth antigen-binding moiety capable of binding said antigen; and (2) a fourth partially double-stranded nucleic acid linked to the fourth antigen-binding moiety, wherein said fourth partially-double-stranded nucleic acid is configured to form said circular nucleic acid product when said first antigen binder, said second antigen binder, said third antigen binder, and said fourth antigen binder form a complex with said antigen. In some embodiments, the third-partially double-stranded nucleic acid comprises: a third proximal nucleic acid linked to the third antigen binding moiety and a third distal nucleic acid hybridized to the third proximal nucleic acid, wherein the third proximal nucleic acid comprises an unhybridized 3′ or 5′ overhanging end configured to hybridize to an unhybridized 3′ or 5′ overhanging end of said first distal nucleic acid of said first antigen binder said third distal nucleic acid comprises an unhybridized 3′ or 5′ overhanging end configured to hybridize to an unhybridized 3′ or 5′ overhanging end of said proximal nucleic acid of said second antigen binder; said second proximal nucleic acid comprises an unhybridized 3′ or 5′ overhanging end configured to hybridize to an unhybridized 3′ or 5′ overhanging end of said third distal nucleic acid, and said second distal nucleic acid comprises an unhybridized 3′ or 5′ overhanging end configured to hybridize to an unhybridized 3′ or 5′ overhanging end of said first proximal nucleic acid; or (2) the fourth partially double-stranded nucleic acid comprises: a fourth proximal nucleic acid linked to the third antigen binding moiety and a fourth distal nucleic acid hybridized to the third proximal nucleic acid, wherein said fourth proximal nucleic acid comprises an unhybridized 3′ or 5′ overhanging end configured to hybridize to an unhybridized 3′ or 5′ overhanging end of said first proximal nucleic acid of said first antigen binder and said fourth distal nucleic acid comprises an unhybridized 3′ or 5′ overhanging end configured to hybridize to an unhybridized 3′ or 5′ overhanging end of said proximal nucleic acid of said third antigen binder, wherein said third partially double-stranded nucleic acid of said third antigen binder comprises a third proximal nucleic acid linked to the third antigen binding moiety and a third distal nucleic acid hybridized to the third proximal nucleic acid, wherein the third proximal nucleic acid comprises an unhybridized 3′ or 5′ overhanging end configured to hybridize to an unhybridized 3′ or 5′ overhanging end of said fourth distal nucleic acid and said third distal nucleic acid comprises an unhybridized 3′ or 5′ overhanging end configured to hybridize to an unhybridized 3′ or 5′ overhanging end of said second proximal nucleic acid. In some embodiments, said plurality of antigen binders has a collectively higher affinity for said antigen compared to a plurality of antigen binders where each binds independently or compared to a plurality of antigen binders where each is not linked via hybridization during binding. In some embodiments, at least one antigen-binder of said plurality of antigen-binders is immobilized on a solid surface. In some embodiments, said solid surface comprises a bead. In some embodiments, said plurality of antigen binders comprises an antigen-binding moiety comprising an antigen-binding molecule. In some embodiments, the method further comprises, prior to (a), immobilizing said antigen on a solid surface. In some embodiments, said solid surface is a bead. In some embodiments, said antigen is immobilized on said solid surface via an antigen-binding biomolecule, wherein said antigen-binding biomolecule is optionally via hybridization to an oligonucleotide immobilized on said solid surface. In some embodiments, said antigen is not immobilized on a surface. In some embodiments, said plurality of antigen binders is not immobilized on a surface. In some embodiments, (a) is a homogenous binding procedure in solution. In some embodiments, the method further comprises detecting a second antigen, wherein (a) further comprises contacting said second antigen with a second plurality of antigen binders to form a second complex, wherein said second plurality of antigen binders is collectively capable of forming a second circular nucleic acid product upon simultaneous binding of said second plurality of antigen binders to an antigen. In some embodiments, said second plurality of antigen binders comprises at least: (i) a first antigen binder capable of binding said second antigen comprising: (1) a first antigen-binding moiety capable of binding said second antigen and (2) a first partially double-stranded nucleic acid linked to said first antigen-binding moiety capable of binding said second antigen; and (ii) a second antigen binder comprising: (1) a second antigen-binding moiety capable of binding said second antigen; and (2) a second partially double-stranded nucleic acid linked to said second antigen-binding moiety capable of binding said second antigen; wherein said first antigen binder and said second antigen binder are configured to template production of a second circular nucleic acid product from at least said first partially double-stranded nucleic acid linked to said first antigen-binding moiety and said second partially double-stranded nucleic acid linked to said second antigen-binding moiety. In some embodiments, said first partially double-stranded nucleic acid linked to said first antigen-binding moiety, said second partially double-stranded nucleic acid linked to the second antigen-binding moiety, or said second partially double-stranded nucleic acid linked to said second antigen-binding moiety capable of binding said second antigen comprises a sequence capable of uniquely identifying said antigen or said second antigen. In some embodiments, said first circular nucleic acid product uniquely identifies a molecule of said antigen or said second circular product uniquely identifies a molecule of said second antigen. In some embodiments, said first partially double-stranded nucleic acid linked to said first antigen-binding moiety, said second partially double-stranded nucleic acid linked to the second antigen-binding moiety, or said second partially double-stranded nucleic acid linked to said second antigen-binding moiety comprises a barcode, a sample index, or a unique molecular identifier (UMI). In some embodiments, said plurality of antigen binders or said second plurality of antigen binders comprises an antigen binding moiety comprising an antibody. In some embodiments, said antibody is a polyclonal antibody. In some embodiments, said antibody is a monoclonal antibody. In some embodiments, the method does not comprise contacting said antigen with a blocking oligonucleotide prior to said contacting to said plurality of antigen binders or said second plurality of antigen binders.

In some aspects, the present disclosure provides a composition comprising plurality of antigen-binders for detecting an antigen, wherein said plurality of antigen binders are collectively capable of forming a circular nucleic acid product upon binding of said plurality of antigen binders to said antigen, wherein said plurality of antigen binders comprise at least: (i) a first antigen binder comprising: (1) a first antigen-binding moiety capable of binding said antigen and (2) first partially double-stranded nucleic acid linked to said first antigen-binding moiety; and (ii) a second antigen-binder comprising: (1) a second antigen-binding moiety capable of binding said antigen; and (2) a second partially double-stranded nucleic acid linked to said second antigen-binding moiety wherein said first partially double-stranded nucleic acid and said second partially double-stranded nucleic acid are configured to template production of said circular nucleic acid product via at least said first partially double-stranded nucleic acid and said second double-stranded nucleic acid. In some embodiments, said first-partially double-stranded nucleic acid comprises: (i) a first proximal nucleic acid linked to said first antigen binder comprising a common hybridization region and an unhybridized overhanging 5′ end; and (ii) a first distal nucleic acid comprising a common hybridization region and an unhybridized overhanging 5′ end, wherein said first distal nucleic acid is hybridized to said first proximal nucleic acid via said common hybridization region having said free unhybridized 5′ end; and said second-partially double-stranded nucleic acid comprises: (i) said second proximal nucleic acid linked to said second antigen binder comprising a common hybridization region and an unhybridized overhanging 5′ end; and (ii) said second distal nucleic acid comprising a common hybridization region and an unhybridized overhanging 5′ end, wherein said second distal nucleic acid is hybridized to said second proximal nucleic acid via said common hybridization region having said unhybridized overhanging 5′ end; wherein said free unhybridized 5′ end of said first proximal nucleic acid is configured to bind to said unhybridized overhanging 5′ end of said second distal nucleic acid, and said unhybridized overhanging 5′ end of said second proximal nucleic acid is configured to bind to said unhybridized overhanging 5′ end of said first distal nucleic acid. In some embodiments, said first-partially double-stranded nucleic acid comprises: (i) a first proximal nucleic acid linked to said first antigen binder comprising a common hybridization region and an unhybridized overhanging 3′ end; and (ii) a first distal nucleic acid comprising a common hybridization region and an unhybridized overhanging 3′ end, wherein said first distal nucleic acid is hybridized to said first proximal nucleic acid via said common hybridization region having said unhybridized overhanging 3′ end; and said second-partially double-stranded nucleic acid comprises: (i) a second proximal nucleic acid linked to said second antigen binder comprising a common hybridization region and an unhybridized overhanging 3′ end; and (ii) a second distal nucleic acid comprising a common hybridization region and unhybridized overhanging end, wherein said second distal nucleic acid is hybridized to said second proximal nucleic acid via said common hybridization region having said unhybridized overhanging 3′ end; wherein said unhybridized overhanging 3′ end of said first proximal nucleic acid is configured to bind to said unhybridized overhanging 3′ end of said second distal nucleic acid, and said unhybridized overhanging 3′ end of said second proximal nucleic acid is configured to bind to said unhybridized overhanging 3′ end of said first distal nucleic acid. In some embodiments, wherein a length of said unhybridized 3′ or 5′ overhanging end of said first proximal nucleic acid and a length of said unhybridized 3′ or 5′ overhanging end of said second proximal nucleic acid are not equal. In some embodiments, the composition further comprises contacting said antigen and said plurality of antigen binders with a tunable partially double-stranded nucleic acid, wherein said tunable partially double-stranded nucleic acid is configured to form said circular nucleic acid when said tunable partially double-stranded nucleic acid contacts at least said first partially double-stranded nucleic acid and said second partially double-stranded nucleic acid. In some embodiments, said plurality of antigen binders further comprises (iii) a third antigen-binder comprising: (1) a third antigen-binding moiety capable of binding said antigen; and (2) a third partially double-stranded nucleic acid linked to said third antigen-binding moiety, wherein said third partially-double-stranded nucleic acid linked to said third antigen-binding moiety is configured to form said circular nucleic acid product when said first antigen binder and said second antigen binder form a complex with said antigen. In some embodiments, the third-partially double-stranded nucleic acid comprises: a third proximal nucleic acid linked to the third antigen binding moiety and a third distal nucleic acid hybridized to the third proximal nucleic acid, wherein the second proximal nucleic acid comprises an unhybridized 3′ or 5′ overhanging end configured to hybridize to an unhybridized 3′ or 5′ overhanging end of said first proximal nucleic acid of said first antigen binder. In some embodiments, said plurality of antigen binders has a collectively higher affinity for said antigen compared to a plurality of antigen binders comprising only two antigen binders. In some embodiments, at least one antigen-binder of said plurality of antigen-binders is immobilized on a solid surface. In some embodiments, said solid surface comprises a bead. In some embodiments, said plurality of antigen binders comprises an antigen-binding moiety comprising an antigen-binding molecule. In some embodiments, said antigen is immobilized on said solid surface via an antigen-binding biomolecule. In some embodiments, said antigen is not immobilized on a surface. In some embodiments, said plurality of antigen binders is not immobilized on a surface. In some embodiments, the composition further comprises a second plurality of antigen binders configured to form a second complex, wherein said second plurality of antigen binders is collectively capable of forming a second circular nucleic acid product upon simultaneous binding of said second plurality of antigen binders to an antigen. In some embodiments, said second plurality of antigen binders comprises at least: (i) a first antigen binder capable of binding said second antigen comprising: (1) a first antigen-binding moiety capable of binding said second antigen and (2) a first partially double-stranded nucleic acid linked to said first antigen-binding moiety capable of binding said second antigen; and (ii) a second antigen binder comprising: (1) a second antigen-binding moiety capable of binding said second antigen; and (2) a second partially double-stranded nucleic acid linked to said second antigen-binding moiety capable of binding said second antigen; wherein said first antigen binder and said second antigen binder are configured to template production of a second circular nucleic acid product from at least said first partially double-stranded nucleic acid linked to said first antigen-binding moiety and said second partially double-stranded nucleic acid linked to said second antigen-binding moiety. In some embodiments, said first partially double-stranded nucleic acid linked to said first antigen-binding moiety, said second partially double-stranded nucleic acid linked to the second antigen-binding moiety, or said second partially double-stranded nucleic acid linked to said second antigen-binding moiety capable of binding said second antigen comprises a sequence capable of binding said antigen or said second antigen. In some embodiments, said first circular nucleic acid product uniquely identifies a molecule of said antigen or said second circular product uniquely identifies a molecule of said second antigen. In some embodiments, said first partially double-stranded nucleic acid linked to said first antigen-binding moiety, said second partially double-stranded nucleic acid linked to the second antigen-binding moiety, or said second partially double-stranded nucleic acid linked to said second antigen-binding moiety capable of binding said second antigen comprises a first barcode or a unique molecular identifier (UMI). In some embodiments, said plurality of antigen binders or said second plurality of antigen binders comprises an antigen binding moiety comprising an antibody. In some embodiments, said antibody is a polyclonal antibody. In some embodiments, said antibody is a monoclonal antibody. In some embodiments, the composition does not comprise contacting said antigen with a blocking oligonucleotide prior to said contacting to said plurality of antigen binders or said second plurality of antigen binders.

In some aspects, the present disclosure provides a method of detecting an antigen, comprising: (a) contacting an antigen with a plurality of antigen binders to form a complex comprising said antigen bound to antigen binders of said plurality of antigen binders, wherein, during said contacting, said antigen binders comprise at least: (i) a first antigen binder comprising: (1) a first antigen-binding moiety capable of binding said antigen; and (2) first partially double-stranded nucleic acid linked to said first antigen-binding moiety; and (ii) a second antigen-binder comprising: (1) a second antigen-binding moiety capable of binding said antigen; and (2) a second partially double-stranded nucleic acid linked to said second antigen-binding moiety wherein said first partially double-stranded nucleic acid and said second partially double-stranded nucleic acid do not contain overhanging ends configured to hybridize to one another; wherein said first partially double-stranded nucleic acid and said second partially double-stranded nucleic acid are configured to template production of a linear nucleic acid product via at least said first partially double-stranded nucleic acid and said second double-stranded nucleic acid when in the presence of a single-stranded template nucleic acid configured to hybridize to an overhanging end of said first partially double-stranded nucleic acid and said second partially-double-stranded nucleic acid; (b) introducing said single-stranded template nucleic acid to said complex; and (c) producing said linear nucleic acid product from at least said first partially double-stranded nucleic acid and said second-double-stranded nucleic acid. In some embodiments, said first-partially double-stranded nucleic acid comprises: (i) a first proximal nucleic acid linked to said first antigen binder comprising a common hybridization region and an optional unhybridized overhanging end; and (ii) a first distal nucleic acid comprising a common hybridization region and an unhybridized overhanging end, wherein said first distal nucleic acid is hybridized to said first proximal nucleic acid via said common hybridization region; and said second-partially double-stranded nucleic acid comprises: (i) said second proximal nucleic acid linked to said second antigen binder comprising a hybridization region and an optional unhybridized overhanging end; and (ii) said second distal nucleic acid comprising a common hybridization region and an unhybridized overhanging end, wherein said second distal nucleic acid is hybridized to said second proximal nucleic acid via said common hybridization region. In some embodiments, said first-partially double-stranded nucleic acid comprises: (i) a first proximal nucleic acid linked to said first antigen binder comprising a common hybridization region and an optional unhybridized overhanging end; and (ii) a first distal nucleic acid comprising a common hybridization region and an unhybridized overhanging 5′ end, wherein said first distal nucleic acid is hybridized to said first proximal nucleic acid via said common hybridization region; and said second-partially double-stranded nucleic acid comprises: (i) said second proximal nucleic acid linked to said second antigen binder comprising a hybridization region and an optional unhybridized overhanging end; and (ii) said second distal nucleic acid comprising a common hybridization region and an unhybridized overhanging 5′ end, wherein said second distal nucleic acid is hybridized to said second proximal nucleic acid via said common hybridization region In some embodiments, said first-partially double-stranded nucleic acid comprises: (i) a first proximal nucleic acid linked to said first antigen binder comprising a common hybridization region and an optional unhybridized overhanging end; and (ii) a first distal nucleic acid comprising a common hybridization region and an unhybridized overhanging 3′ end, wherein said first distal nucleic acid is hybridized to said first proximal nucleic acid via said common hybridization region; and said second-partially double-stranded nucleic acid comprises: (i) a second proximal nucleic acid linked to said second antigen binder comprising a common hybridization region; and (ii) a second distal nucleic acid comprising a common hybridization region and an unhybridized overhanging 3′ end, wherein said second distal nucleic acid is hybridized to said second proximal nucleic acid via said common hybridization. In some embodiments, single-stranded template nucleic acid is configured to hybridize to an unhybridized overhanging end of said first partially double-stranded nucleic acid and said second partially double-stranded nucleic acid. In some embodiments, the method further comprises detecting said linear nucleic acid product comprising at least said first distal nucleic acid and said second distal nucleic acid, thereby detecting said antigen. In some embodiments, the method further comprises, prior to (a), immobilizing said antigen on a solid surface. In some embodiments, said solid surface is a bead. In some embodiments, said antigen is immobilized on said solid surface via an antigen-binding biomolecule or via hybridization to an oligonucleotide immobilized on said solid surface. In some embodiments, said antigen is not immobilized on a surface. In some embodiments, said plurality of antigen binders is not immobilized on a surface. In some embodiments, (a) is a homogenous binding procedure in solution. In some embodiments, the method further comprises detecting a second antigen, wherein (a) further comprises contacting said second antigen with a second plurality of antigen binders to form a second complex, wherein said second plurality of antigen binders is collectively capable of forming a linear nucleic acid product upon simultaneous binding of said second plurality of antigen binders to an antigen in the presence of a single-stranded template nucleic acid. In some embodiments, said first partially double-stranded nucleic acid linked to said first antigen-binding moiety, said second partially double-stranded nucleic acid linked to the second antigen-binding moiety, or said second partially double-stranded nucleic acid linked to said second antigen-binding moiety capable of binding said second antigen comprises a barcode or a unique molecular identifier (UMI). In some embodiments, said plurality of antigen binders or said second plurality of antigen binders comprises an antigen binding moiety comprising an antibody. In some embodiments, the method does not comprise contacting said antigen with a blocking oligonucleotide prior to said contacting to said plurality of antigen binders or said second plurality of antigen binders. In some embodiments, said plurality of antigen binders further comprises (iii) a third antigen binder comprising: (1) a third antigen-binding moiety capable of binding said antigen; and (2) a third partially double-stranded nucleic acid linked to the third antigen-binding moiety, wherein said third partially-double-stranded nucleic acid is configured to form said linear nucleic acid product when said first antigen binder, said second antigen binder, and said third antigen binder form a complex with said antigen in the presence of a second single-stranded template nucleic acid that binds to said partially double-stranded nucleic acid of said second antigen binder; or (iv) a fourth antigen binder comprising (1) a fourth antigen-binding moiety capable of binding said antigen; and (2) a fourth partially double-stranded nucleic acid linked to the fourth antigen-binding moiety, wherein said fourth partially-double-stranded nucleic acid is configured to form said linear nucleic acid product when said first antigen binder, said second antigen binder, said third antigen binder, and said fourth antigen binder form a complex with said antigen in the presence of a third single-stranded template nucleic acid that binds to said partially double-stranded nucleic acids of said third antigen binder.

In some aspects, the present disclosure provides a composition of detecting an antigen, comprising: (a) an antigen with a plurality of antigen binders to form a complex comprising said antigen bound to antigen binders of said plurality of antigen binders, said antigen binders comprise at least: (i) a first antigen binder comprising: (1) a first antigen-binding moiety capable of binding said antigen; and (2) first partially double-stranded nucleic acid linked to said first antigen-binding moiety; and (ii) a second antigen-binder comprising: (1) a second antigen-binding moiety capable of binding said antigen; and (2) a second partially double-stranded nucleic acid linked to said second antigen-binding moiety wherein said first partially double-stranded nucleic acid and said second partially double-stranded nucleic acid do not contain overhanging ends configured to hybridize to one another; wherein said first partially double-stranded nucleic acid and said second partially double-stranded nucleic acid are configured to template production of a linear nucleic acid product via at least said first partially double-stranded nucleic acid and said second double-stranded nucleic acid when in the presence of a single-stranded template nucleic acid configured to hybridize to an overhanging end of said first partially double-stranded nucleic acid and said second partially-double-stranded nucleic acid; (b) introducing or contacting said single-stranded template nucleic acid to said complex. In some embodiments, said first-partially double-stranded nucleic acid comprises: (i) a first proximal nucleic acid linked to said first antigen binder comprising a common hybridization region and an optional unhybridized overhanging end; and (ii) a first distal nucleic acid comprising a common hybridization region and an unhybridized overhanging end, wherein said first distal nucleic acid is hybridized to said first proximal nucleic acid via said common hybridization region; and said second-partially double-stranded nucleic acid comprises: (i) said second proximal nucleic acid linked to said second antigen binder comprising a hybridization region and an optional unhybridized overhanging end; and (ii) said second distal nucleic acid comprising a common hybridization region and an unhybridized overhanging end, wherein said second distal nucleic acid is configured to hybridize to said second proximal nucleic acid via said common hybridization region. In some embodiments, said first-partially double-stranded nucleic acid comprises: (i) a first proximal nucleic acid linked to said first antigen binder comprising a common hybridization region and an optional unhybridized overhanging end; and (ii) a first distal nucleic acid comprising a common hybridization region and an unhybridized overhanging 5′ end, wherein said first distal nucleic acid is configured to hybridize to said first proximal nucleic acid via said common hybridization region; and said second-partially double-stranded nucleic acid comprises: (i) said second proximal nucleic acid linked to said second antigen binder comprising a hybridization region and an optional unhybridized overhanging end; and (ii) said second distal nucleic acid comprising a common hybridization region and an unhybridized overhanging 5′ end, wherein said second distal nucleic acid is configured to hybridize to said second proximal nucleic acid via said common hybridization region. In some embodiments, said first-partially double-stranded nucleic acid comprises: (i) a first proximal nucleic acid linked to said first antigen binder comprising a common hybridization region and an optional unhybridized overhanging end; and (ii) a first distal nucleic acid comprising a common hybridization region and an unhybridized overhanging 3′ end, wherein said first distal nucleic acid is configured to hybridize to said first proximal nucleic acid via said common hybridization region; and said second-partially double-stranded nucleic acid comprises: (i) a second proximal nucleic acid linked to said second antigen binder comprising a common hybridization region; and (ii) a second distal nucleic acid comprising a common hybridization region and an unhybridized overhanging 3′ end, wherein said second distal nucleic acid is configured to hybridize to said second proximal nucleic acid via said common hybridization. In some embodiments, single-stranded template nucleic acid is configured to hybridize to an unhybridized overhanging end of said first partially double-stranded nucleic acid and said second partially double-stranded nucleic acid. In some embodiments, the composition further comprises a solid surface on which said antigen can be immobilized. In some embodiments, said solid surface is a bead. In some embodiments, said solid surface further comprises an antigen-binding biomolecule immobilized on said solid surface. In some embodiments, said composition does not comprise a solid surface. In some embodiments, said solid surface does not comprise said plurality of antigen binders immobilized thereon. In some embodiments, said composition is provided in solution. In some embodiments, the composition further comprises a second plurality of antigen binders configured to form a second complex, wherein said second plurality of antigen binders is collectively capable of forming a linear nucleic acid product upon simultaneous binding of said second plurality of antigen binders to an antigen in the presence of a single-stranded template nucleic acid. In some embodiments, said first partially double-stranded nucleic acid linked to said first antigen-binding moiety, said second partially double-stranded nucleic acid linked to the second antigen-binding moiety, or said second partially double-stranded nucleic acid linked to said second antigen-binding moiety capable of binding said second antigen comprises a barcode or a unique molecular identifier (UMI). In some embodiments, said plurality of antigen binders or said second plurality of antigen binders comprises an antigen binding moiety comprising an antibody. In some embodiments, the composition does not a blocking oligonucleotide. In some embodiments, said plurality of antigen binders further comprises (iii) a third antigen binder comprising: (1) a third antigen-binding moiety capable of binding said antigen; and (2) a third partially double-stranded nucleic acid linked to the third antigen-binding moiety, wherein said third partially-double-stranded nucleic acid is configured to form said linear nucleic acid product when said first antigen binder, said second antigen binder, and said third antigen binder form a complex with said antigen in the presence of a second single-stranded template nucleic acid that binds to said partially double-stranded nucleic acid of said second antigen binder; or (iv) a fourth antigen binder comprising (1) a fourth antigen-binding moiety capable of binding said antigen; and (2) a fourth partially double-stranded nucleic acid linked to the fourth antigen-binding moiety, wherein said fourth partially-double-stranded nucleic acid is configured to form said linear nucleic acid product when said first antigen binder, said second antigen binder, said third antigen binder, and said fourth antigen binder form a complex with said antigen in the presence of a third single-stranded template nucleic acid that binds to said partially double-stranded nucleic acids of said third antigen binder.

In some aspects, the present disclosure provides a method of detecting an antigen, comprising: (a) contacting an antigen with a plurality of antigen binders and a tunable nucleic acid to form a complex comprising said antigen bound to antigen binders of said plurality of antigen binders, wherein, during said contacting, said antigen binders and said tunable nucleic acid comprise at least: (i) a first antigen binder comprising: (1) a first antigen-binding moiety capable of binding said antigen; and (2) first partially double-stranded nucleic acid linked to said first antigen-binding moiety; and (ii) a second antigen-binder comprising: (1) a second antigen-binding moiety capable of binding said antigen; and (2) a second partially double-stranded nucleic acid linked to said second antigen-binding moiety; and (iii) a tunable nucleic acid, comprising a third partially double-stranded nucleic acid, which is not conjugated to an antigen-binding moiety; wherein said first partially double-stranded nucleic acid, said second partially double-stranded nucleic acid, and said tunable nucleic acid are configured to template production of a linear nucleic acid product via said tunable nucleic acid when said first and second antigen binder bind to said antigen; and (b) producing said linear nucleic acid product from at least said first partially double-stranded nucleic acid and said second-double-stranded nucleic acid. In some embodiments, the method further comprises contacting said antigen with: (iii) a third antigen binder comprising: (1) a third antigen-binding moiety capable of binding said antigen; and (2) a third partially double-stranded nucleic acid linked to the third antigen-binding moiety, wherein said third partially-double-stranded nucleic acid is configured to form said linear nucleic acid product when said first antigen binder, said second antigen binder, wherein said tunable nucleic acid is configured to bridge said partially double-stranded nucleic acids of said first and said second antigen binder, or said second and said third antigen binder.

In some aspects, the present disclosure provides a composition for detecting an antigen, comprising: (a) a plurality of antigen binders and a tunable nucleic acid configured to form a complex comprising said antigen bound to antigen binders of said plurality of antigen binders, wherein said antigen binders and said tunable nucleic acid comprise at least: (i) a first antigen binder comprising: (1) a first antigen-binding moiety capable of binding said antigen; and (2) first partially double-stranded nucleic acid linked to said first antigen-binding moiety; and (ii) a second antigen-binder comprising: (1) a second antigen-binding moiety capable of binding said antigen; and (2) a second partially double-stranded nucleic acid linked to said second antigen-binding moiety; and (iii) a tunable nucleic acid, comprising a third partially double-stranded nucleic acid, which is not conjugated to an antigen-binding moiety; wherein said first partially double-stranded nucleic acid, said second partially double-stranded nucleic acid, and said tunable nucleic acid are configured to template production of a linear nucleic acid product via said tunable nucleic acid when said first and second antigen binder bind to said antigen; and In some embodiments, the composition further comprises said antigen with: (iii) a third antigen binder comprising: (1) a third antigen-binding moiety capable of binding said antigen; and (2) a third partially double-stranded nucleic acid linked to the third antigen-binding moiety, wherein said third partially-double-stranded nucleic acid is configured to form said linear nucleic acid product when said first antigen binder, said second antigen binder, wherein said tunable nucleic acid bridges said partially double-stranded nucleic acids of said first and said second antigen binder, or said second and said third antigen binder.

In some aspects, the present disclosure provides a method of detecting an antigen, comprising: (a) contacting an antigen with a plurality of antigen binders to form a complex comprising said antigen bound to antigen binders of said plurality of antigen binders, wherein, during said contacting, said antigen binders comprise at least: (i) a first antigen binder comprising: (1) a first antigen-binding moiety capable of binding said antigen; and (2) first partially double-stranded nucleic acid linked to said first antigen-binding moiety wherein said first-partially double-stranded nucleic acid comprises: (A) a first proximal nucleic acid linked to said first antigen binder comprising a common hybridization region and an unhybridized overhanging end; and
(B) a first distal nucleic acid comprising a common hybridization region and an unhybridized overhanging end, wherein said first distal nucleic acid is configured to hybridize to said first proximal nucleic acid via said common hybridization region; and (ii) a second antigen-binder comprising: (1) a second antigen-binding moiety capable of binding said antigen; and (2) a second partially double-stranded nucleic acid linked to said second antigen-binding moiety wherein said second-partially double-stranded nucleic acid comprises: (A) said second proximal nucleic acid linked to said second antigen binder comprising a hybridization region and an unhybridized overhanging end; and (B) said second distal nucleic acid comprising a common hybridization region and an unhybridized overhanging end, wherein said second distal nucleic acid is configured to hybridize to said second proximal nucleic acid via said common hybridization region wherein said first partially double-stranded nucleic acid and said second partially double-stranded nucleic acid do not contain overhanging ends configured to hybridize to one another; wherein said first partially double-stranded nucleic acid and said second partially double-stranded nucleic acid are configured to template production of a circular nucleic acid product via at least said first partially double-stranded nucleic acid and said second double-stranded nucleic acid when in the presence of at least a first and a second single-stranded template nucleic acid; (b) introducing said first and second single-stranded template nucleic acids to said complex; and (c) producing said linear nucleic acid product from at least said first partially double-stranded nucleic acid and said second-double-stranded nucleic acid. In some embodiments, said plurality of antigen binders further comprises: (iii) a third antigen binder comprising: (1) a third antigen-binding moiety capable of binding said antigen; and (2) a third partially double-stranded nucleic acid linked to the third antigen-binding moiety, wherein said third partially-double-stranded nucleic acid is configured to form said circular nucleic acid product when said first antigen binder, said second antigen binder, and said third antigen binder form a complex with said antigen in the presence of a third single-stranded template nucleic acid, wherein said third-partially double-stranded nucleic acid comprises: (A) a third proximal nucleic acid linked to said first antigen binder comprising a common hybridization region and an optional unhybridized overhanging end; and (B) a third distal nucleic acid comprising a common hybridization region and an unhybridized overhanging end, wherein said first distal nucleic acid is configured to hybridize to said first proximal nucleic acid via said common hybridization region.

In some aspects, the present disclosure provides a composition of detecting an antigen, comprising: (a) a plurality of antigen binders configured to form a complex comprising said antigen bound to antigen binders of said plurality of antigen binders, wherein said antigen binders comprise at least: (i) a first antigen binder comprising: (1) a first antigen-binding moiety capable of binding said antigen; and (2) first partially double-stranded nucleic acid linked to said first antigen-binding moiety wherein said first-partially double-stranded nucleic acid comprises: (A) a first proximal nucleic acid linked to said first antigen binder comprising a common hybridization region and an unhybridized overhanging end; and (B) a first distal nucleic acid comprising a common hybridization region and an unhybridized overhanging end, wherein said first distal nucleic acid is configured to hybridize to said first proximal nucleic acid via said common hybridization region; and (ii) a second antigen-binder comprising: (1) a second antigen-binding moiety capable of binding said antigen; and (2) a second partially double-stranded nucleic acid linked to said second antigen-binding moiety wherein said second-partially double-stranded nucleic acid comprises: (A) said second proximal nucleic acid linked to said second antigen binder comprising a hybridization region and an unhybridized overhanging end; and (B) said second distal nucleic acid comprising a common hybridization region and an unhybridized overhanging end, wherein said second distal nucleic acid is configured to hybridize to said second proximal nucleic acid via said common hybridization region wherein said first partially double-stranded nucleic acid and said second partially double-stranded nucleic acid do not contain overhanging ends configured to hybridize to one another; wherein said first partially double-stranded nucleic acid and said second partially double-stranded nucleic acid are configured to template production of a circular nucleic acid product via at least said first partially double-stranded nucleic acid and said second double-stranded nucleic acid when in the presence of at least a first and a second single-stranded template nucleic acid; and (b) said first and second single-stranded template nucleic acids. In some embodiments, said plurality of antigen binders further comprises: (iii) a third antigen binder comprising: (1) a third antigen-binding moiety capable of binding said antigen; and (2) a third partially double-stranded nucleic acid linked to the third antigen-binding moiety, wherein said third partially-double-stranded nucleic acid is configured to form said circular nucleic acid product when said first antigen binder, said second antigen binder, and said third antigen binder form a complex with said antigen in the presence of a third single-stranded template nucleic acid, wherein said third-partially double-stranded nucleic acid comprises: (A) a third proximal nucleic acid linked to said first antigen binder comprising a common hybridization region and an optional unhybridized overhanging end; and (B) a third distal nucleic acid comprising a common hybridization region and an unhybridized overhanging end, wherein said first distal nucleic acid is configured to hybridize to said first proximal nucleic acid via said common hybridization region.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present disclosure will be obtained by reference to the accompanying drawings of which:

FIG. 1A depicts a detection complex intermediate that can allow for an improved method of multiplex detection of protein analytes. This method utilizes a set of at least 2 coincident antigen binder-nucleic acid conjugates (e.g. 3 antibodies are shown in FIG. 1A as the Y-shaped structures labeled Ab1, Ab2, Ab3) that bind a same protein analyte target to produce a templated reporter structure which can signify a binding event. The antigen binder-nucleic acid conjugates can be prepared via any suitable method (e.g. maleimide conjugation, click chemistry conjugation)

Each of the antigen binders used for detection is covalently linked to a proximal single-stranded nucleic acid molecule (e.g. to a 5′ end of the proximal nucleic acid molecule) which comprises: (i) a unique barcode sequence (A₁, A₂, A₃); and (ii) a proximal 3′ adapter sequence (T₁, T₂, T₃). The proximal nucleic acid molecule covalently attached to each antigen binder is in turn hybridized to a distal single-stranded nucleic acid molecule via a region complementary to the unique barcode sequence of the proximal nucleic acid molecule (A1′, A2′, A3′); the distal single stranded nucleic acid molecule comprises: (i) a 5′ region comprising the region complementary to the unique barcode sequence of the proximal nucleic acid molecule (A1′, A2′, A3′); and (ii) a distal 3′ adapter sequence (T₁′, T₂′, T₃′).

For each antigen binder-nucleic acid conjugate (Ab1, Ab2, Ab3): (a) the proximal 3′ adapter sequence (e.g. T₃ for Ab3) is configured to hybridize to the distal 3′ adapter sequence (e.g. T₃′ for Ab2) of one of the antigen binders of the coincident antigen binder set; and (b) the distal 3′ adapter sequence (e.g. T₁′ for Ab3) is configured to hybridize to the proximal adapter sequence of an antigen binder of the coincident antigen binder set that is other than the antigen binder in (a) (e.g. T₁ of Ab1). As a result of this configuration, when a single protein analyte brings antigen binders of the coincident antigen binder set (Ab1, Ab2, Ab3) into proximity, a circular nucleic acid nanostructure can be assembled via hybridization; this product comprises A₁′, T₁′, A3′, T₃′, A₂′, and T₂′ (e.g. all of the barcodes from the antigen binders). If the distal single-stranded nucleic acids are provided as 5′ phosphorylated molecules or T4 polynucleotide kinase or ampligase is provided alongside them, the annealed circular nucleic acid product can be filled in with polymerase and ligase to produce a continuous unique single-stranded circular nucleic acid molecule from the distal single-stranded nucleic acids that serves as a reporter of the binding event (alternatively, if retention of the distal nucleic acid molecule is not required and the proximal 3′ adapter sequence is provided to directly abut the 5′ ends of the distal nucleic acids, polymerase may not be required). After ligation, each continuous unique single-stranded circular nucleic acid molecule can be isolated from background primers by exonuclease treatment (which digests all non-circular DNA) and the sequence detected by sequencing (e.g. next-generation sequencing).

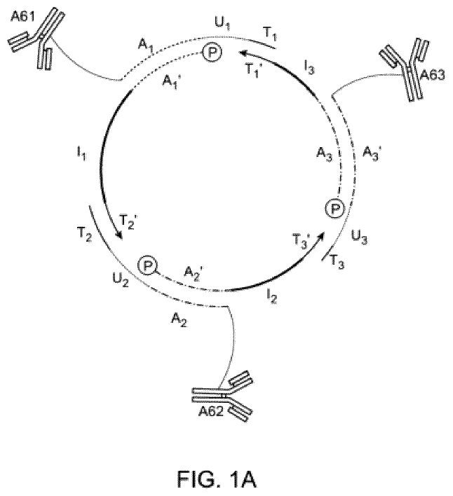

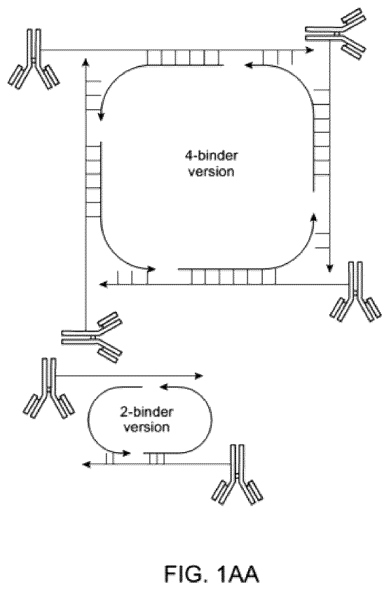

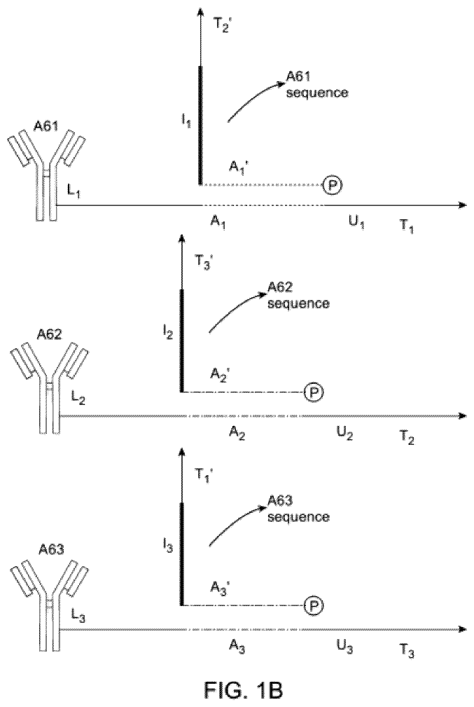

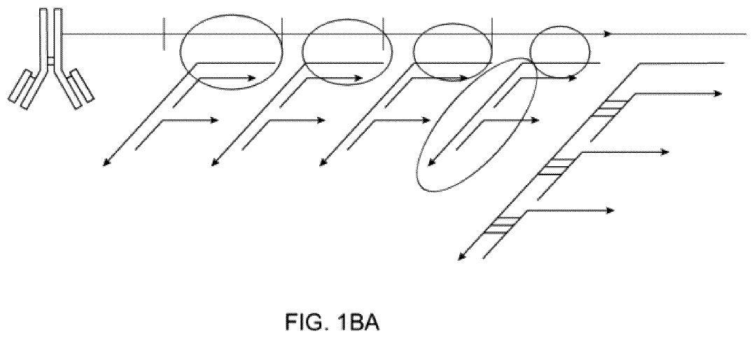

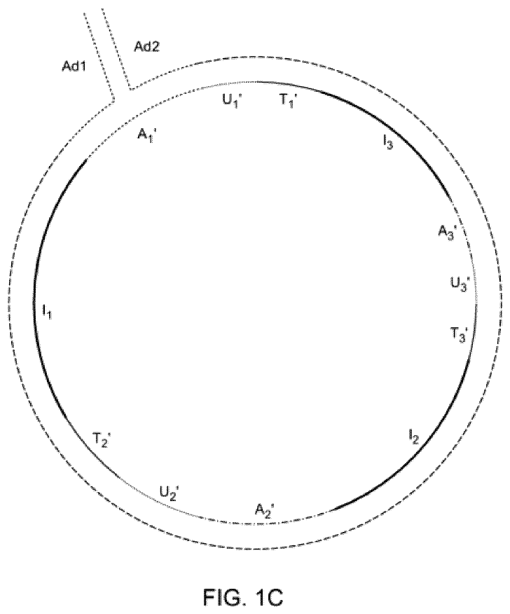

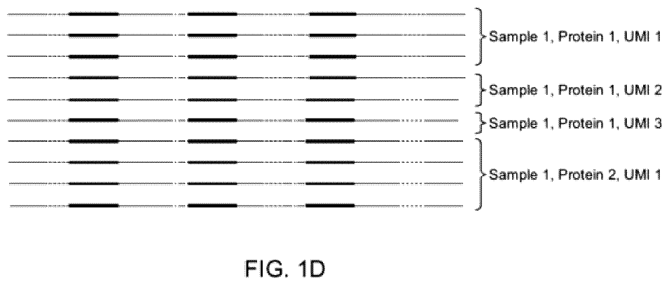

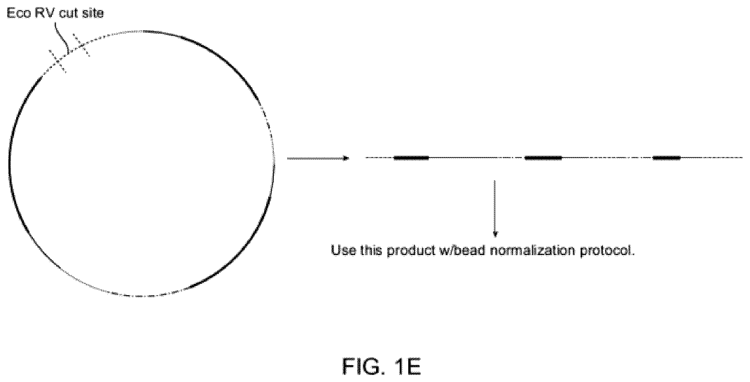

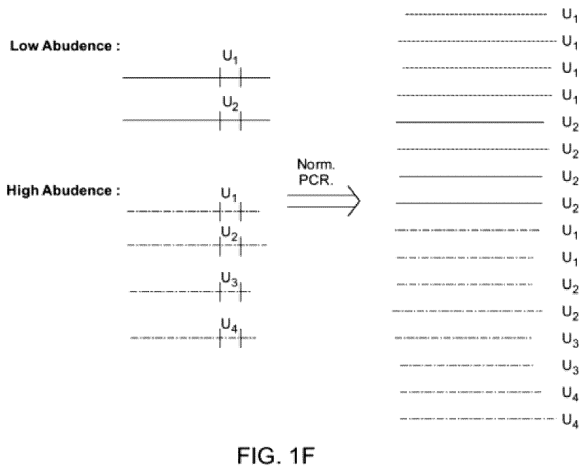

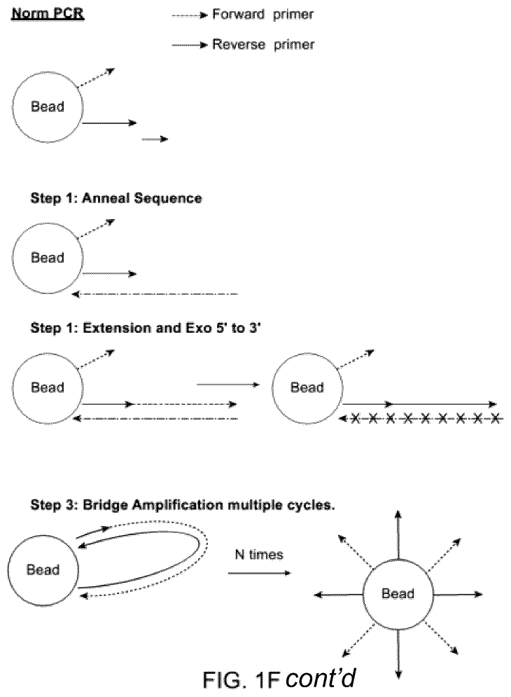

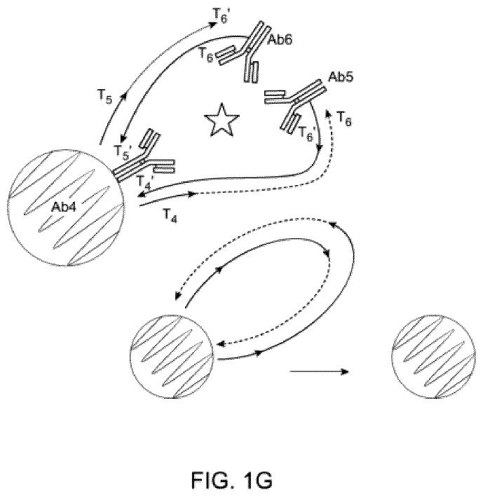

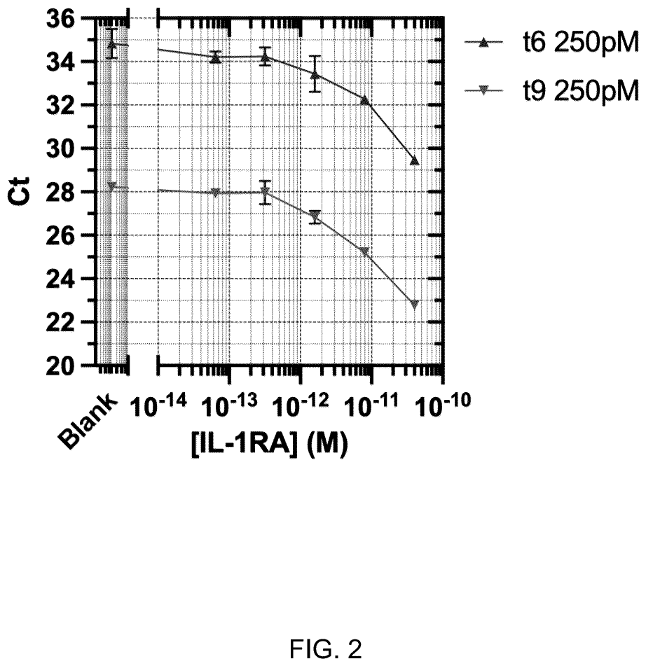

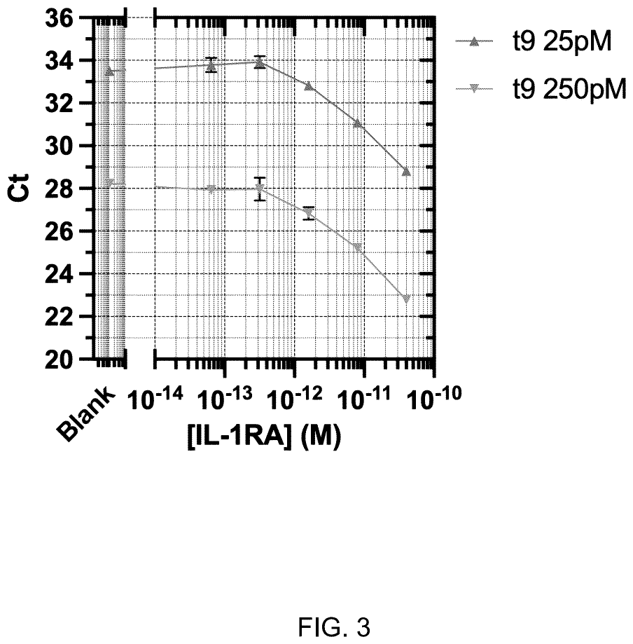

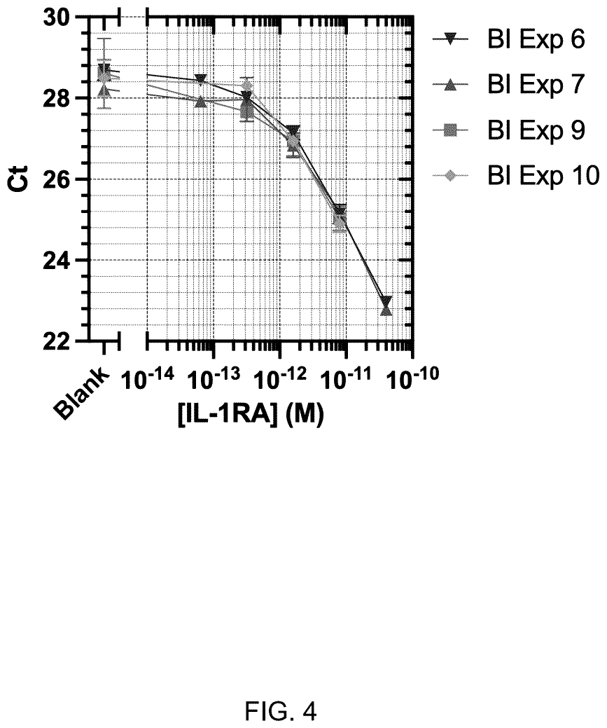

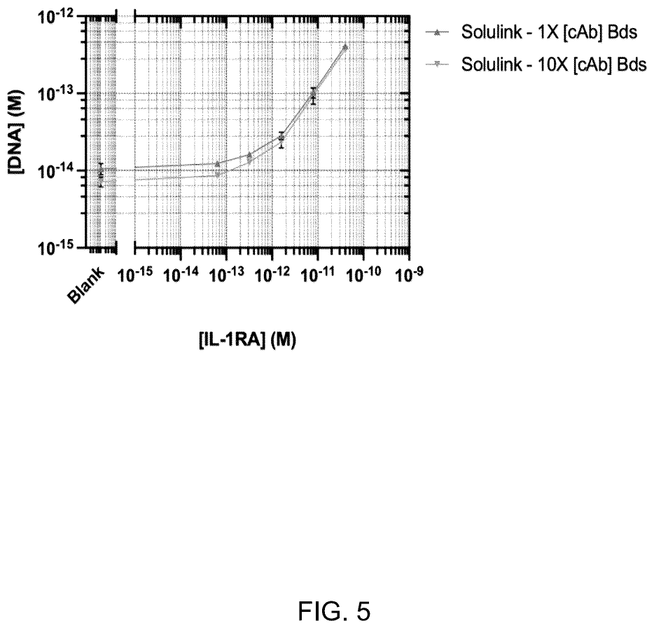

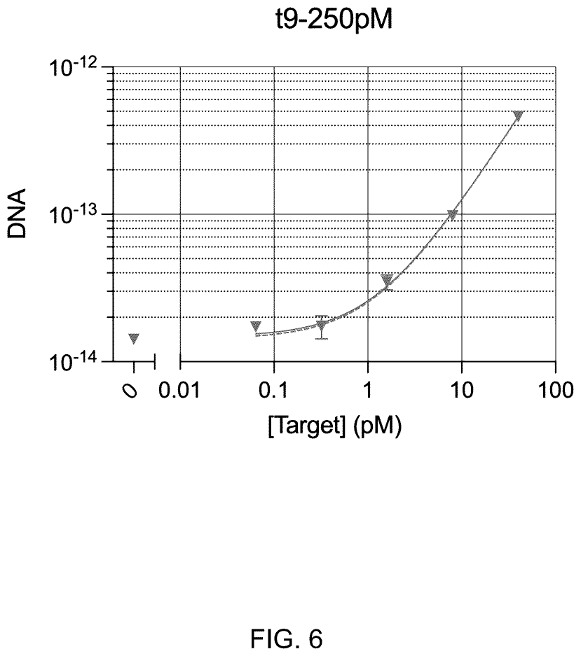

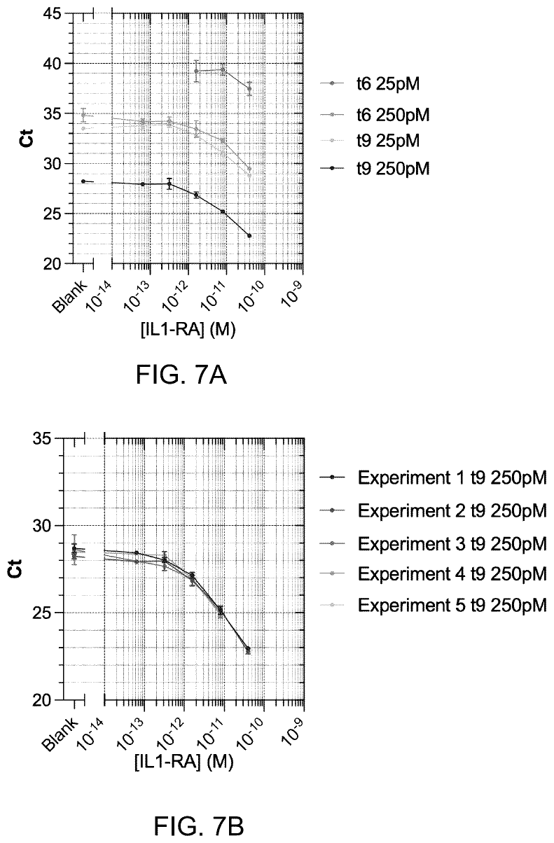

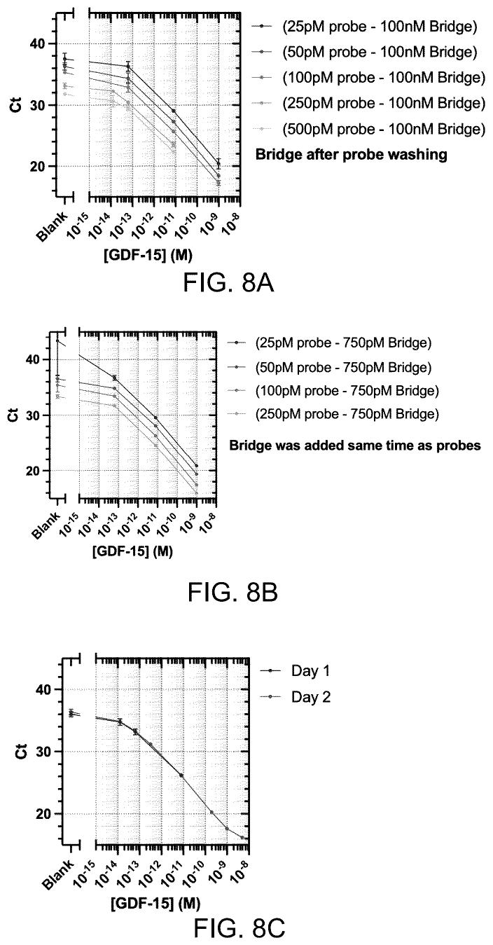

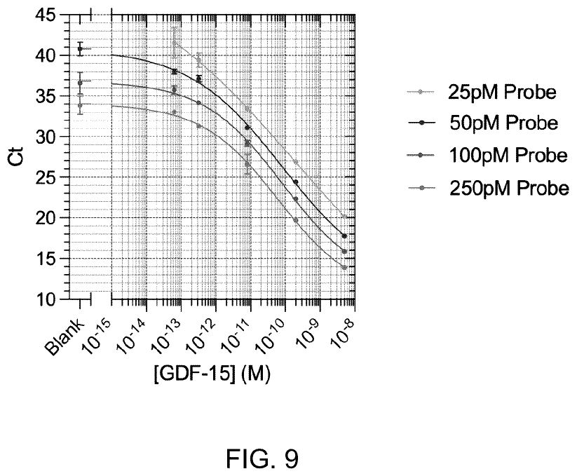

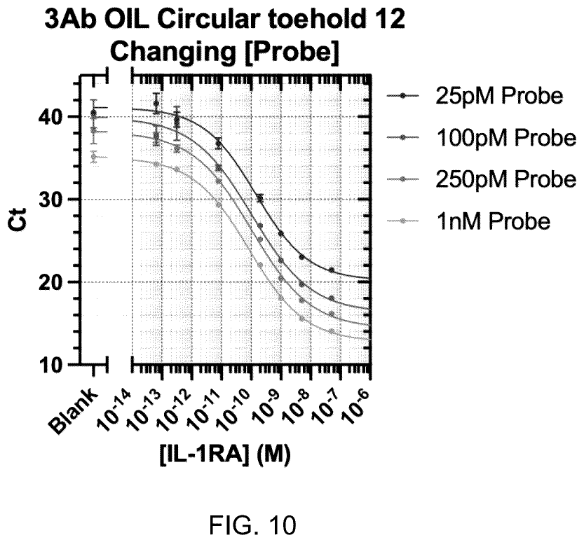

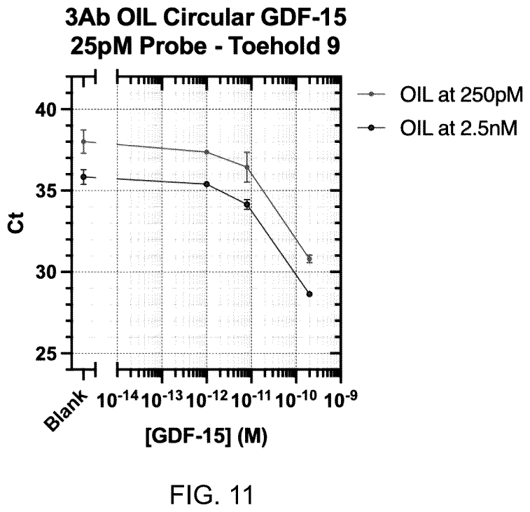

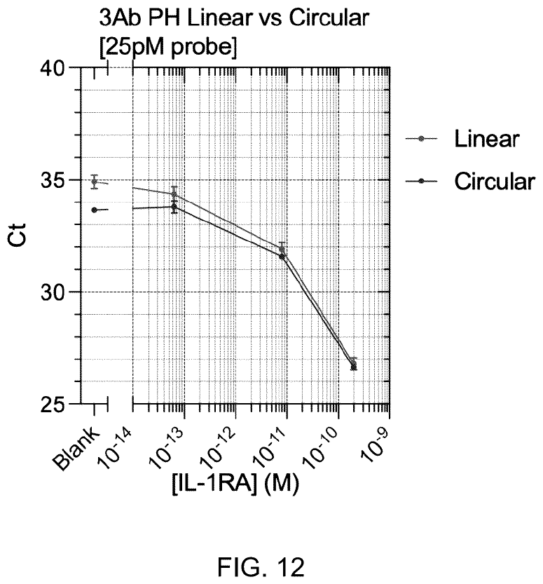

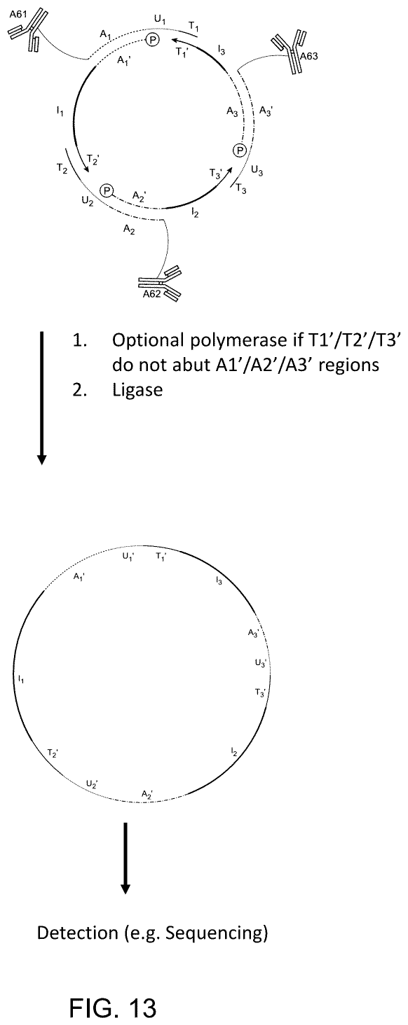

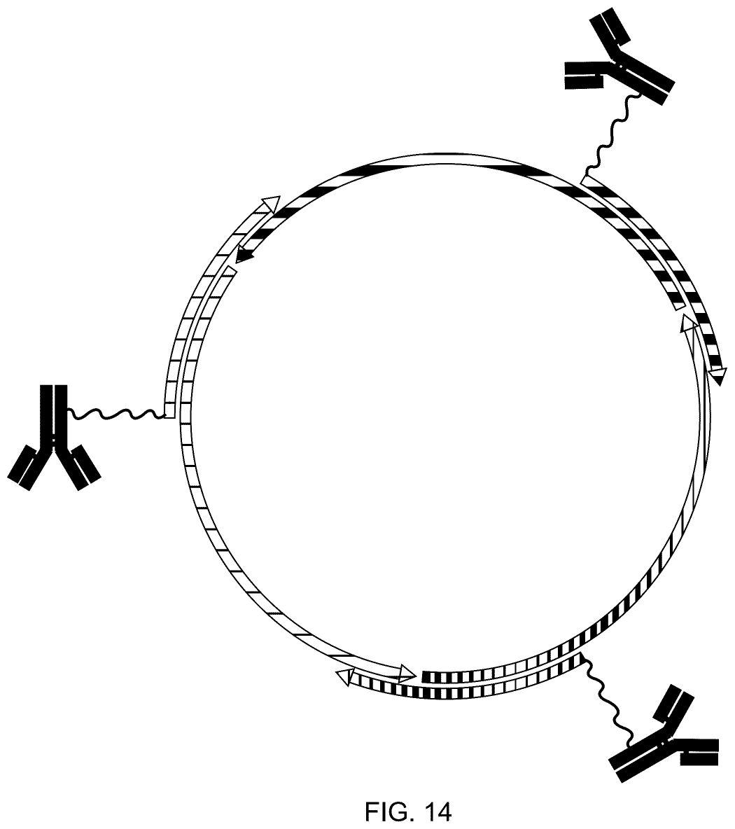

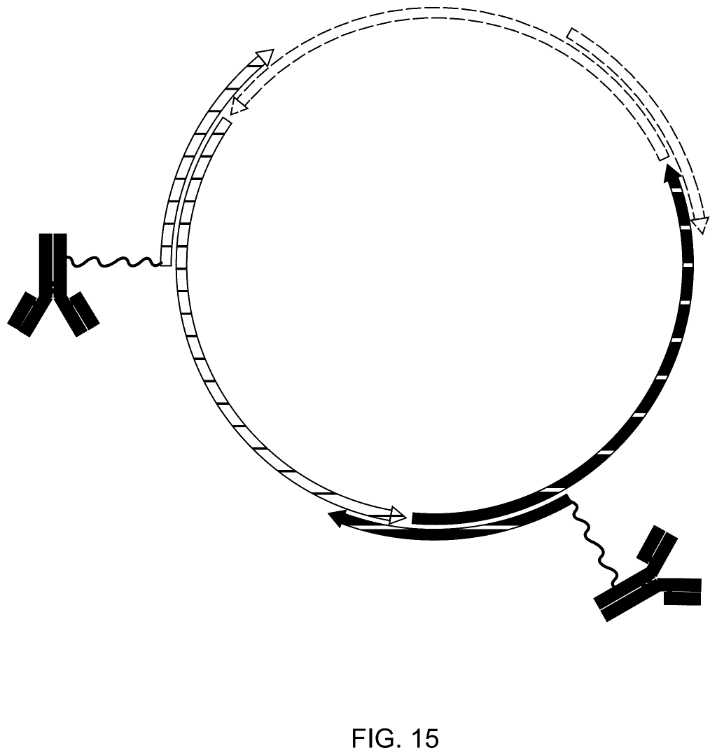

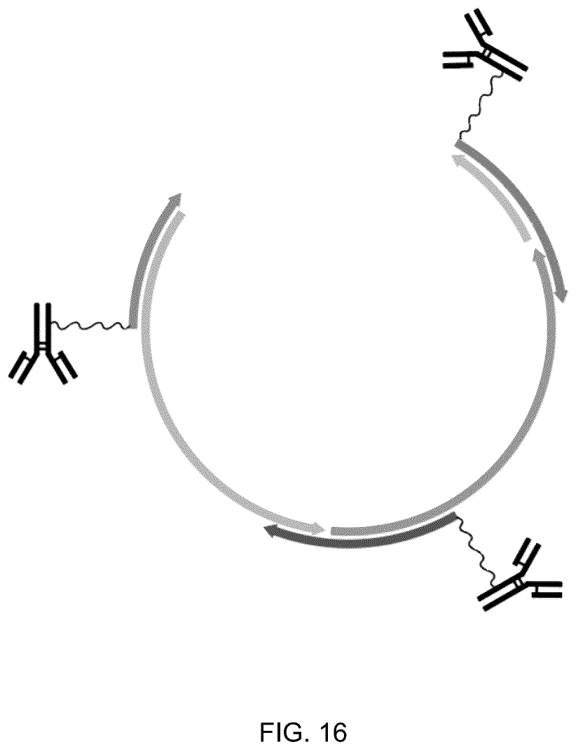

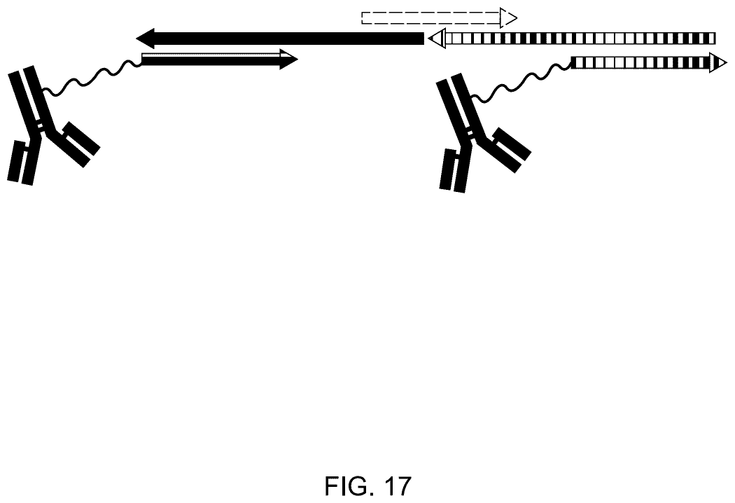

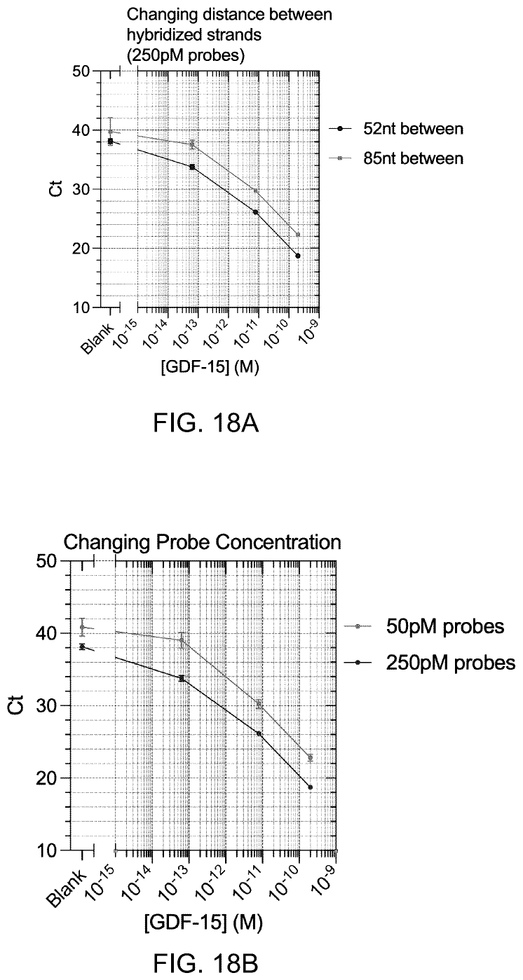

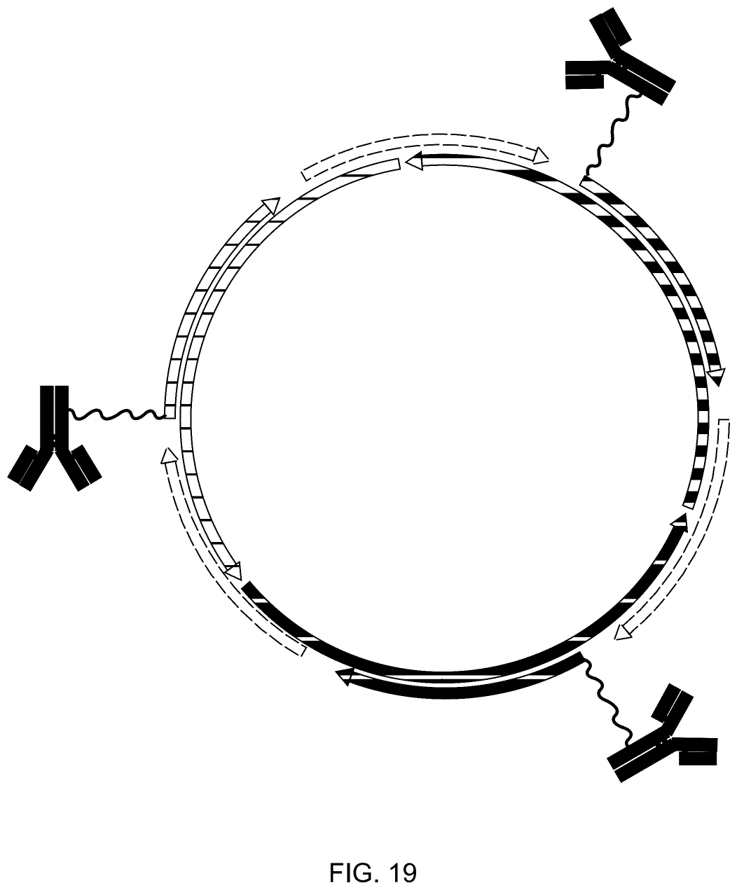

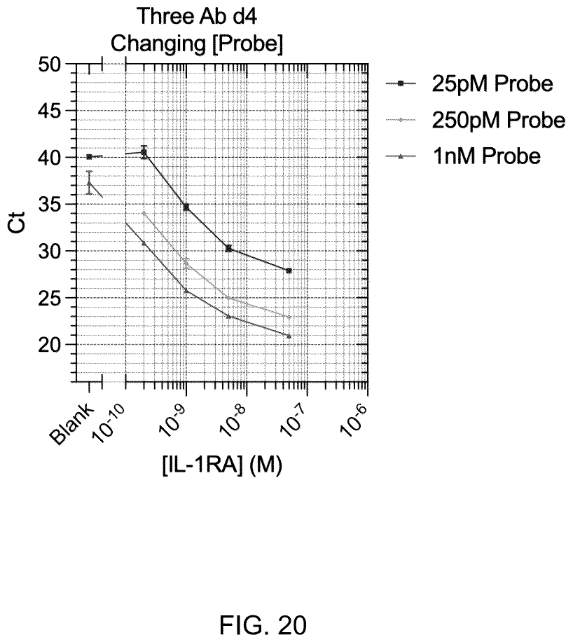

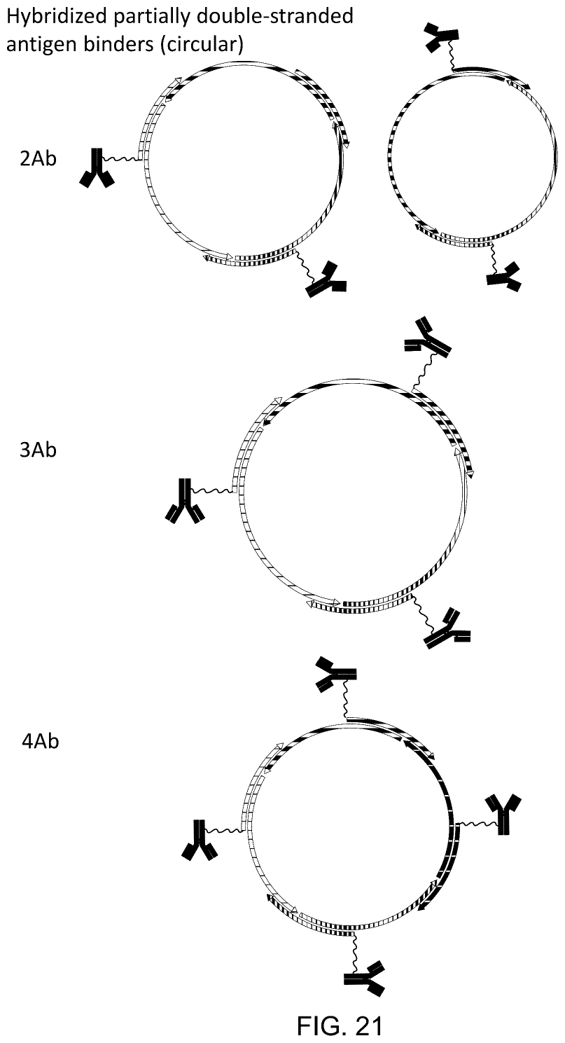

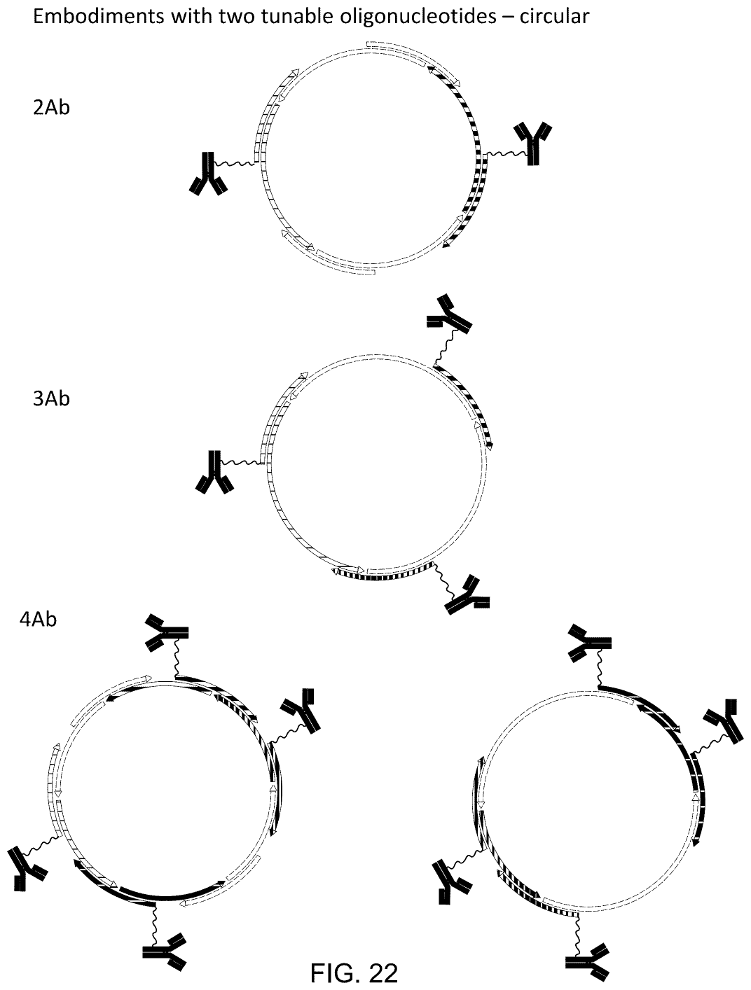

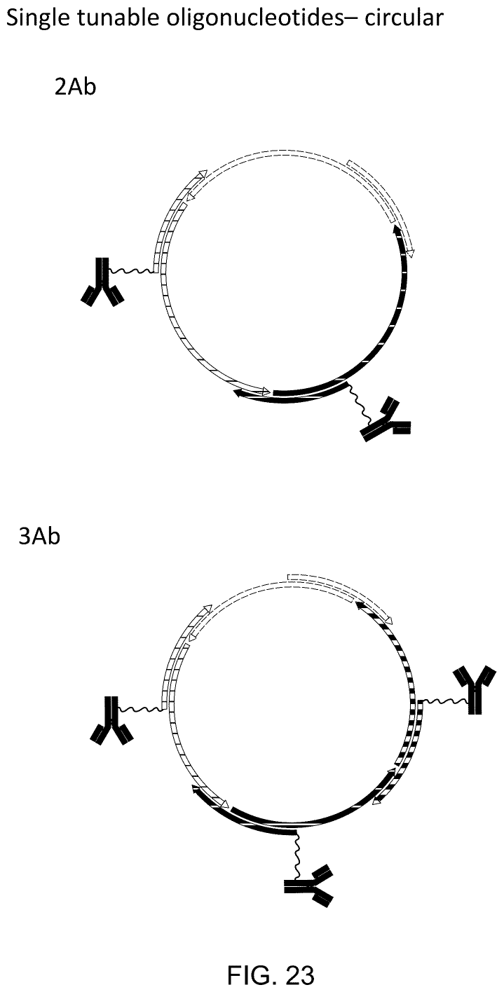

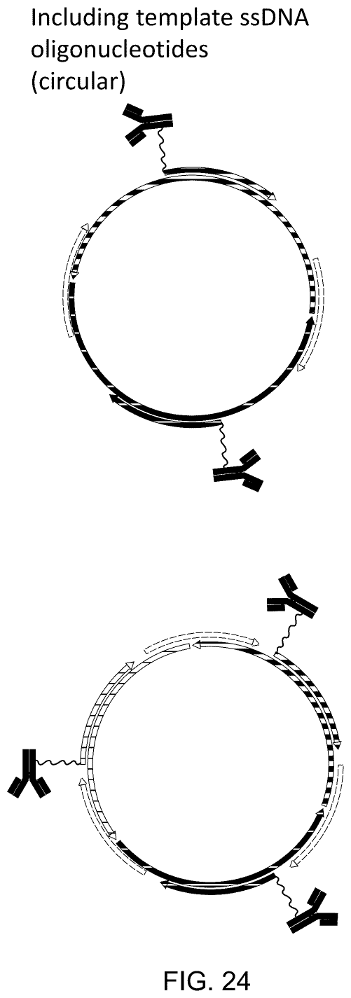

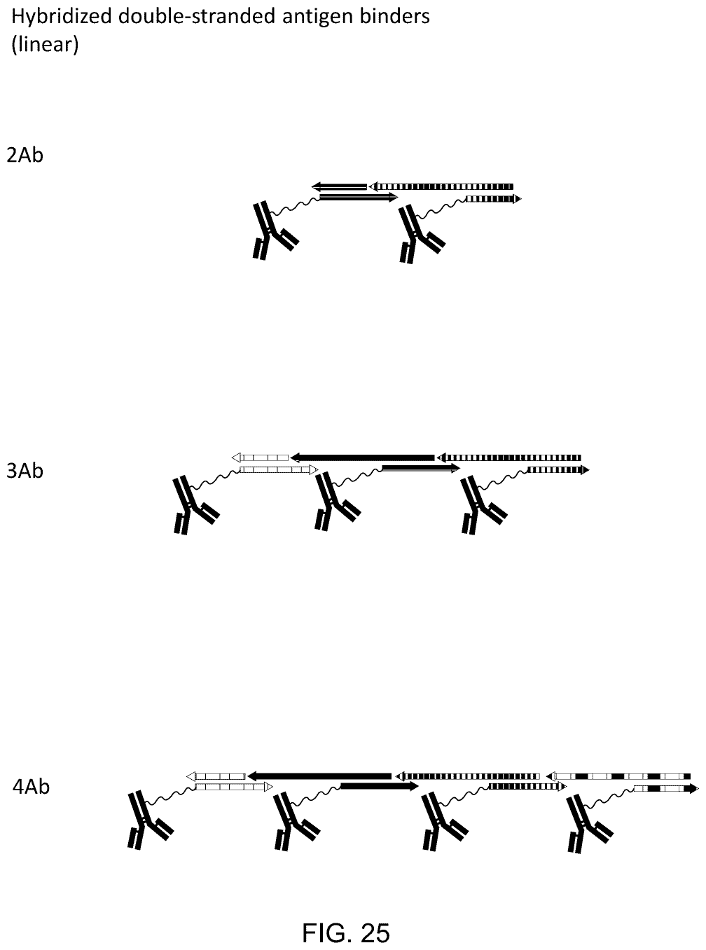

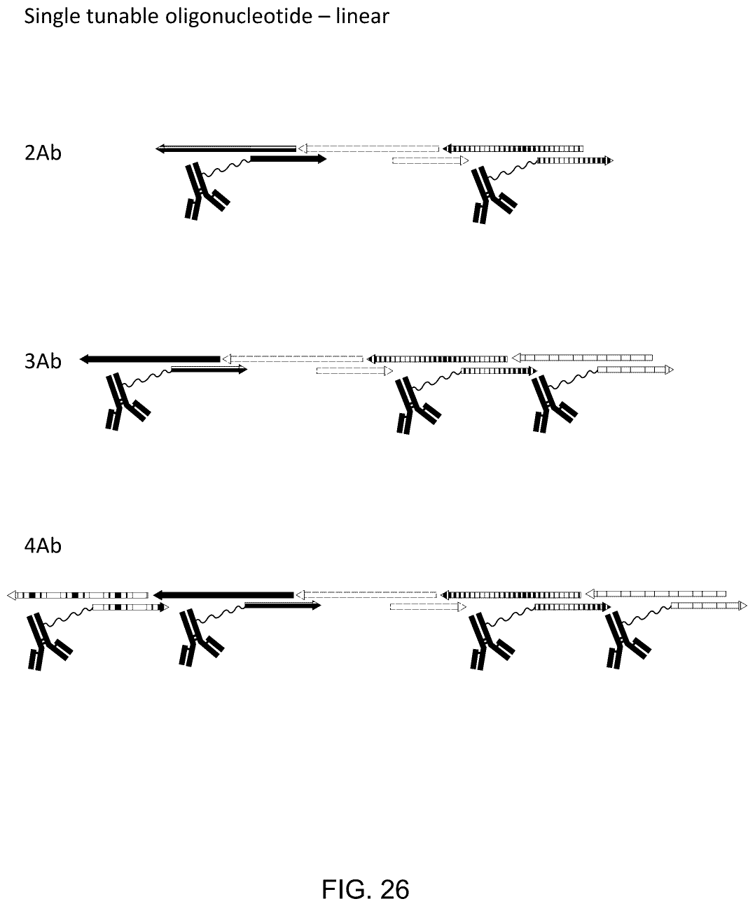

The method shown in FIG. 1A can be generalized to N analytes, provided that sufficient diversity of barcodes covalently attached to the antigen binders (e.g. Ab1, Ab2, Ab3) are provided. In some cases, a unique combination of two or more of A₁, A₂, or A₃ uniquely identifies an individual target. In some cases, A₁, A₂, or A₃ are universal, and a unique combination of two or more of T₁, T₂, or T3 can uniquely identify an individual target. Additionally, a separate motif can be added to solely identify a target.

The method shown in FIG. 1A can also be generalized to a multiplexed analysis of N different protein analyte-containing samples. As the total number of samples or analytes that can be detected by the assay depends on the number of unique combinations of barcodes or indexes (where the number of unique sequences allowing distinction of the unique entities is equal to n*(N)\hat{ }(1/n) to demultiplex N different samples or analytes. In this case, additional barcode or index sequences (e.g. I₁, I₂, I₃), can be provided between the unique barcode sequences (A1, A2, A3) and the distal 3′ adapter sequence (T₁, T₂, T₃). In some embodiments, the index sequences (e.g. I₁, I₂, I₃). In some embodiments, the index sequences (e.g. the combination of I₁, I₂, I₃) can be used to identify N different samples (e.g., for three index sequences, I1, I2, I3, 3*(N)\hat{ }(⅓) unique sequences can be used). In some embodiments, the indexes are added in any location on a distal oligo of the partially double-stranded nucleic acid linked to the antibody.

The method shown in FIG. 1A can also be generalized to detect various dynamic ranges of individual molecules of protein analyte. In some cases, unique molecular identifiers (UMIs) can be incorporated (U₁, U₂, U₃) between the unique barcode sequences (A1, A2, A3) and the proximal 3′ adapter sequence (T₁, T₂, T₃). In the situation where UMIs are provided on the single-stranded nucleic acids linked to the antigen binders, each unique combination of two or more UMIs can signify an individual molecule of analyte when detected downstream (e.g. by sequencing or qPCR).

When the proximity ligation method of FIG. 1A is used to detect multiple distinct protein analytes, and the circular nucleic acid products are to be detected in a same sequencing reaction, an open question is how to ensure that low-abundance and high-abundance protein analytes can be detected with similar accuracy (since the abundance of the circular products will differ). In this case, improved accuracy can be achieved by performing an amplification system (“Norm. PCR”) that results in similar abundance for circular nucleic acid products that result from low- and high-abundance analytes (see FIG. 1F). After normalization, unique combinations of UMIs are used to detect individual abundance of protein analytes; for low-abundant protein analytes each unique UMI combination may be present in multiple copies, whereas for higher-abundance protein analytes the representation of unique UMI combinations approaches a single copy. This can allow for cross-target and cross-sample normalization of DNA concentrations to greatly facilitate the process of library pooling for NGS.

FIG. 1AA is a schematic showing that the scheme of FIG. 1A can be generalized to 2 or 4 antigen binders.

FIG. 1B depicts example organization for three antigen binder-nucleic acid conjugates usable with the detection complex depicted in FIG. 1A.

Ab1, Ab2, and Ab3 signify three antigen-binders configured to form the structure depicted in FIG. 1A, which are all directed against a same protein analyte. These antigen-binders can comprise antigen binders (e.g. full-length IgG molecules), fragments of antigen binders (e.g. Fab fragments), derivatives of antigen binders (e.g. scFvs), or aptamers. If the antigen-binders are antibodies, the antigen binders can be monoclonal or polyclonal in derivation.

Ab1, Ab2, and Ab3 are each conjugated to a proximal nucleic acid that can comprise (a) L₁, L₂, and L₃; (b) A₁, A₂, and A₃; (c) U₁, U₂, and U₃; or (d) T₁, T₂, and T₃.

L₁, L₂, and L₃ signify regions of nucleic acid (or a non-nucleic acid molecule) used as a linker to distance the antigen binders from the tripartite conjugate depicted in FIG. 1A. In some cases, nucleic acid regions can comprise from one up to 40-60 nucleotides of a polyadenine sequence or a polythymidine sequence. In some cases, a non-nucleic acid molecule can comprise a polymeric linker such as polyethylene glycol. In some cases, the linkers are configured to control the effective concentration of the individual reporter molecule components such that they associate when all the antigen binder-nucleic acid conjugates bind a single target.

A₁, A₂, and A₃ signify regions of nucleic acid (e.g. DNA) that comprise at least a unique nucleic acid sequence. In some cases, the antigen binder-nucleic acid conjugates are envisioned for use in a multiplexed pool where there are multiple sets of coincident antigen binder-nucleic acid conjugates, wherein each coincident antigen binder-nucleic acid conjugates set is directed against a different protein analyte. In this case, A₁, A₂, and A₃ encode a sequence that uniquely identifies the protein target that the antigen binder-nucleic acid conjugate set is directed against. In some cases, A₁ is up to 40-50 nucleotides in length. In some cases, A₁ comprises one or more of: a reverse primer sequence, a forward primer sequence, or a restriction enzyme cut site (e.g. an EcoRV cut site), or a reverse complement of any of these. In some cases, the forward and reverse primers comprise sequences complementary to that of Illumina adapter primers. In some cases, A₂ and A₃ are shorter sequence of up to 20-30 nucleotides that also uniquely identify the protein target they are directed against.

U₁, U₂, and U₃ signify regions of nucleic acid comprising unique molecular identifiers (UMIs) that differ for each individual molecule of antigen binder conjugated to the nucleic acid. When detected via sequencing of a molecule such as that depicted in FIG. 1A, each unique combination of U₁, U₂, and U₃ can signify an individual binding event and thus an individual molecule of protein analyte in a protein analyte-containing sample. In some cases U₁, U₂, and U₃ comprise up to 12 nucleotides in length. In some cases, U1, U2, and U3 are optional. T₁, T₂, and T₃ signify adapter (or “toehold”) sequences that allow formation of the tripartite structure depicted in FIG. 1A via hybridization. T₁, T₂, and T₃ are configured to hybridize to corresponding sequences T₁′, T₂′, and T₃′ which are on distal nucleic acid molecules associated with separate antigen binder-nucleic acid conjugates to form the reporter nucleic acid structure in FIG. 1A, and can be up to 4-15 nucleotides in length. In some cases, the affinity of binding for the toehold regions can be optimized to control the relative output of different analyte nucleic acid reporters.

Ab1, Ab2, and Ab3 are each associated with a distal nucleic acid, which is associated with the proximal nucleic acid via presence of a corresponding A₁′, A₂′, and A₃′ region in the distal nucleic acid. A₁′, A₂′, and A₃′ signify nucleic acid regions configured to hybridize to A₁, A₂, and A₃. In some cases they are partially or completely complementary to A₁, A₂, and A₃. The distal nucleic acid can comprise (a) A₁′, A₂′, and A₃′; (b) I₁, I₂, and I₃; and (c) T₁′, T₂′, and T₃′. In some cases, the distal nucleic acid can also comprise one or more unique molecular identifier (UMI) sequences. I₁, I₂, and b signify indexing sequences, which can be sample specific indexes. In some cases, I₁ can signify a row number, I₂ can signify a column number, and I₃ can signify a plate number when referring to a multiwell plate of samples containing protein analytes. In some cases, these sequences I₁, I₂, and I₃ can comprise up to 5-10 nucleotides in length.

FIG. 1BA depicts a scheme where, to amplify the signal output from a tripartite binding event such as FIG. 1A, Ab1, Ab2, and Ab3 of FIG. 1A or FIG. 1B are each conjugated to more than one proximal nucleic acid that can comprise additional unique antigen reporter barcodes or UMIs (see e.g. FIG. 1BA).

FIG. 1C depicts a three-component circular nucleic acid product which can signify a binding event, which can be produced by hybridization, optional gap filling, and ligation of the structure depicted in FIG. 1A (optionally T4 polynucleotide kinase treatment if phosphorylated molecules are not provided attached to the antigen binders).

A₁′, A₂′, and A₃′ are described as in FIG. 1A (e.g. as Ab₁′, Ab₂′, and Ab₃′), as are I₁, I₂, and I₃ and T₁′, T₂′, and T₃′. U₁′, U₂′, and U₃′ signify complements of U₁, U₂, and U₃ produced by optional gap filling of the structure depicted in FIG. 1A. As described in FIG. 1B, each unique combination of UMIs U₁, U₂, and U₃ can signify an individual binding event and thus an individual molecule of protein analyte in a protein analyte-containing sample. Thus, sequencing of the circular nucleic acid products produced or sequencing of products derived from the circular nucleic acid products produced can provide a route to identify the total number of protein analytes in the sample (e.g. by identifying the number of unique sequence U₁, U₂, and U₃ products) and each sample can be distinguished by the unique combination of I₁, I₂, and I₃. In FIG. 1C depicted, A₁′ comprises a reverse primer sequence and a forward primer sequence, complementary to that of Illumina adapter primers (Ad1 and Ad2). In some embodiments, Ad1 and Ad2 can be located in any portion of the product generated by the methods disclosed herein.

FIG. 1D depicts a analysis method for the product of FIG. 1C wherein adapter primers Ad1 and Ad2 allows for the production of linear nucleic acid molecules. In some cases, the PCR amplification is optimized so that a multiplexed reaction produces about 10 nM total linear DNA.

FIG. 1E depicts a second analysis method for the product of FIG. 1C wherein A₁′ (or alternatively A₂′ or A₃′) comprises a restriction enzyme site (e.g. an EcoRV cut site), another disruptable site, or polymerase blocking site (e.g., polyA, UV-cleavable linker, or PEG linker) and a reverse primer sequence and a forward primer sequence. In this analysis method, digestion with the restriction enzyme linearizes the circular products according to FIG. 1C and the products are optionally subjected to a bead-based normalization protocol.

FIG. 1F depicts a bead-based normalization procedure compatible with outputs of any of the detection schemes described herein (e.g. FIG. 1A). In this procedure, bridge amplification on a defined number of beads is used to ensure production of equal amounts of circular products for each protein analyte (such a procedure can also be used to normalize other nucleic acid samples comprising indexes). This scheme begins with products of the type depicted in FIG. 1E, where A₁′ (or alternatively A₂′ or A₃′) comprises a restriction enzyme site (e.g. an EcoRV cut site) and a reverse primer sequence and a forward primer sequence; forward and reverse primer sequence are individual to each protein analyte in a multiplex reaction. Linearized products as depicted in FIG. 1E are incubated with a population of beads that comprises subpopulations of equal number directed against circular nucleic acids representative of each protein analyte. Each subpopulation of beads in turn comprises equal loadings of forward and reverse primers specific for circular nucleic acids representative of each protein analyte. Equal loading of the forward/reverse primers can be achieved by a suitable chemical conjugation method (e.g. biotin/streptavidin attachment) of the primers to the beads (where defined loading of biotin or streptavidin or another conjugation moiety on the beads is initially provided). Successive annealing (“step 1”), followed by extension (“step 2”) and exonuclease treatment provides a growing population of amplified products on the beads (“step 3”). Repetition of this process to saturation of the beads, followed by exonuclease treatment and purification of the beads, provides a defined population of amplified products determined by the forward/reverse primer loading on the beads. Removal of the amplified products (or PCR amplification of the products) from the beads provides linear products that can be sequenced by next generation sequencing. This protocol to FIG. 1E can also be used with circular products.

FIG. 1G depicts an alternative detection intermediate to FIG. 1A, in which two antigen binder-nucleic acid conjugates (Ab5 and Ab6) conjugated to single-stranded barcode-bearing nucleic acids are provided, and one antigen binder (Ab4)—is provided attached to a bead (the bead in turn being conjugated to two separate single-stranded barcode-bearing nucleic acids). In this configuration, both the nucleic acids conjugated to antibodies (T₅′, T₆, T₆′, T₄′) and the nucleic acids conjugated to the bead (T₅, T₄) comprise toehold regions that are configured that so when Ab4/Ab5/Ab6 bind a single common analyte, production of a loop double-stranded can be formed given alternating polymerase, denaturation, and polymerase operations. Cleavage of the loop double-stranded nucleic acid product from the beads via a suitable method enables detection of the binding event (e.g. by next-generation sequencing).

FIG. 2 shows a graph of qPCR cycle time against the abundance of IL-1RA using two different overhang or toehold lengths.

FIG. 3 shows a graph of qPCR cycle time against the abundance of IL-1RA using two different concentrations of a probe with an overhang or toehold length of nine nucleotides.

FIG. 4 shows a graph of qPCR cycle time against abundance of TL-1RA over multiple experiments following the same protocol.

FIG. 5 shows a graph of DNA output (from qPCR) against abundance of IL-1RA as a function of the amount of capture antibody loaded onto a magnetic bead.

FIG. 6 shows a graph of DNA output (from qPCR) against abundance of a target using a probe with a toehold of t9 at 250 pM concentration.

FIG. 7A shows a graph of qPCR cycle time against the abundance of IL-1RA using two different overhang or toehold lengths at varying probe concentrations.

FIG. 7B shows a graph of qPCR cycle time against abundance of IL-1RA over multiple experiments following the same protocol.

FIG. 8A shows a graph of qPCR cycle against the abundance of Growth Differentiation Factor 15 (GDF-15) with varying concentrations of a probe with an overhang or toehold length of 9 nucleotides and a high concentration of a tunable partially double-stranded nucleic acid (bridge) added after probe washing.

FIG. 8B shows a graph of qPCR cycle against the abundance of Growth Differentiation Factor 15 (GDF-15) with varying concentrations of a probe with an overhang or toehold length of 9 nucleotides and a tunable partially double-stranded nucleic acid (bridge) added at the same time as the probe.

FIG. 8C shows a graph of qPCR cycle time against abundance of Growth Differentiation Factor 15 (GDF-15) over multiple experiments following the same protocol as in FIG. 8A.

FIG. 9 shows a graph of qPCR cycle time against the abundance of Growth Differentiation Factor 15 (GDF-15) using four different concentrations of a probe with an overhang or toehold length of nine nucleotides.

FIG. 10 shows a graph of qPCR cycle time with various probe concentrations for a probe with a toehold or overhang length of 12 nucleotides with antibodies specific to IL-1RA.

FIG. 11 shows a graph of qPCR cycle time against the abundance of Growth Differentiation Factor 15 (GDF-15) using two different concentrations of a probe with an overhang or toehold length of nine nucleotides.

FIG. 12 compares a linear product versus a circular product formed with 25 pM oligo/probes and antibodies specific for IL-1RA.

FIG. 13 depicts a workflow for formation of a detectable product using the intermediates shown in FIG. 1A.

FIG. 14 depicts formation of a detectable intermediate/product as described in Example 1 or 2.

FIG. 15 depicts formation of a detectable intermediate/product as described in Example 3.

FIG. 16 depicts formation of a detectable intermediate/product as described in Example 4.

FIG. 17 depicts formation of a detectable intermediate/product as described in Example 5.

FIG. 18A shows a graph of qPCR cycle threshold with differing nucleotide distances for linking hybridized strands.

FIG. 18B shows a graph of qPCR cycle threshold with differing probe concentrations.

FIG. 19 depicts formation of a detectable intermediate/product as described in Example 6.

FIG. 20 shows a graph of qPCR cycle threshold with differing probe concentrations.

FIGS. 21, 22, 23, 24, 25, and 26 depict alternate organizations of intermediates that can be used to detect antigens using antigen binders according to methods of the disclosure. FIG. 21 depicts embodiments using partially double-stranded nucleic acid-conjugated antigen binders to produce circular detectable nucleic acids using 2 (“2Ab”), 3 (“3Ab”), or 4 (“4Ab”) antigen binders. FIG. 22 depicts embodiments using at least two tunable nucleotides according to the disclosure and 2 (“2Ab”), 3 (“3Ab”), or 4 (“4Ab”) partially double-stranded nucleic acid-conjugated antigen binders to produce circular detectable nucleic acids. FIG. 23 depicts embodiments using one tunable nucleotide according to the disclosure and 2 (“2Ab”) or 3 (“3Ab”) partially double-stranded nucleic acid-conjugated antigen binders to produce a circular detectable nucleic acid. FIG. 24 depicts embodiments where 2 template oligonucleotides according to the disclosure and 2 partially double-stranded nucleic acid-conjugated antigen binders not configured to hybridize to each other (top panel) or 3 template oligonucleotides according to the disclosure and 3 partially double-stranded nucleic acid-conjugated antigen binders not configured to hybridize to one another are used to produce circular detectable nucleic acids. FIG. 25 depicts embodiments using partially double-stranded nucleic acid-conjugated antigen binders to produce linear detectable nucleic acids using 2 (“2Ab”), 3 (“3Ab”), or 4 (“4Ab”) antigen binders. FIG. 26 depicts embodiments using a single tunable nucleotide according to the disclosure and 2 (“2Ab”), 3 (“3Ab”), or 4 (“4Ab”) partially double-stranded nucleic acid-conjugated antigen binders to produce linear detectable nucleic acids.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Overview

FIG. 1A depicts a detection complex intermediate that can allow for an improved method of multiplex detection of protein analytes (with FIG. 13 depicting an example formation of a detectable product). This method utilizes a set of at least 2 coincident antigen binder-nucleic acid conjugates (e.g. 3 antibodies are shown in FIG. 1A as the Y-shaped structures labeled Ab1, Ab2, Ab3) that bind a same protein analyte target to produce a templated reporter structure which can signify a binding event. The antigen binder-nucleic acid conjugates can be prepared via any suitable method (e.g. maleimide conjugation, click chemistry conjugation)

Each of the antigen binders used for detection is covalently linked to a proximal single-stranded nucleic acid molecule (e.g. to a 5′ end of the proximal nucleic acid molecule) which comprises: (i) a unique barcode sequence (A₁, A₂, A₃); and (ii) a proximal 3′ adapter sequence (T₁, T₂, T₃). The proximal nucleic acid molecule covalently attached to each antigen binder is in turn hybridized to a distal single-stranded nucleic acid molecule via a region complementary to the unique barcode sequence of the proximal nucleic acid molecule (A1′, A2′, A3′); the distal single stranded nucleic acid molecule comprises: (i) a 5′ region comprising the region complementary to the unique barcode sequence of the proximal nucleic acid molecule (A1′, A2′, A3′); and (ii) a distal 3′ adapter sequence (T₁′, T₂′, T₃′).

For each antigen binder-nucleic acid conjugate (Ab1, Ab2, Ab3): (a) the proximal 3′ adapter sequence (e.g. T₃ for Ab3) is configured to hybridize to the distal 3′ adapter sequence (e.g. T₃′ for Ab2) of one of the antigen binders of the coincident antigen binder set; and (b) the distal 3′ adapter sequence (e.g. T₁′ for Ab3) is configured to hybridize to the proximal adapter sequence of an antigen binder of the coincident antigen binder set that is other than the antigen binder in (a) (e.g. T₁ of Ab1). As a result of this configuration, when a single protein analyte brings antigen binders of the coincident antigen binder set (Ab1, Ab2, Ab3) into proximity, a circular nucleic acid nanostructure can be assembled via hybridization; this product comprises A₁′, T₁′, A3′, T₃′, A₂′, and T₂′ (e.g. all of the barcodes from the antigen binders). If the distal single-stranded nucleic acids are provided as 5′ phosphorylated molecules or T₄ polynucleotide kinase or ampligase is provided alongside them, the annealed circular nucleic acid product can be filled in with polymerase and ligase to produce a continuous unique single-stranded circular nucleic acid molecule from the distal single-stranded nucleic acids that serves as a reporter of the binding event (alternatively, if retention of the distal nucleic acid molecule is not required and the proximal 3′ adapter sequence is provided to directly abut the 5′ ends of the distal nucleic acids, polymerase may not be required). After ligation, each continuous unique single-stranded circular nucleic acid molecule can be isolated from background primers by exonuclease treatment (which digests all non-circular DNA) and the sequence detected by sequencing (e.g. next-generation sequencing).

The method shown in FIG. 1A can be generalized to N analytes, provided that sufficient diversity of barcodes covalently attached to the antigen binders (e.g. Ab1, Ab2, Ab3) are provided. In some cases, a unique combination of two or more of A₁, A₂, or A₃ uniquely identifies an individual target. In some cases, A₁, A₂, or A₃ are universal, and a unique combination of two or more of T₁, T₂, or T3 can uniquely identify an individual target.

The method shown in FIG. 1A can also be generalized to a multiplexed analysis of N different protein analyte-containing samples. As the total number of sample or analytes that can be detected by the assay depends on the number of unique combinations of barcodes or indexes (where the number of unique sequences allowing distinction of the unique entities is equal to n*(N)\hat{ }(1/n) to demultiplex N different samples). In this case, additional barcode or index sequences (e.g. I₁, I₂, I₃), can be provided between the unique barcode sequences (A1, A2, A3) and the distal 3′ adapter sequence (T₁, T₂, T₃). In some embodiments, the index sequences (e.g. I₁, I₂, I₃). In some embodiments, the index sequences (e.g. the combination of I₁, I₂, I₃) can be used to identify N different samples (e.g., for three index sequences, I1, I2, I3, 3*(N)\hat{ }(⅓) unique sequences can be used). In some embodiments, the indexes are added in any location on a distal oligo of the partially double-stranded nucleic acid linked to the antibody.

The method shown in FIG. 1A can also be generalized to detect various dynamic ranges of individual molecules of protein analyte. In some cases, unique molecular identifiers (UMIs) can be incorporated (U₁, U₂, U₃) between the unique barcode sequences (A1, A2, A3) and the proximal 3′ adapter sequence (T₁, T₂, T₃). In the situation where UMIs are provided on the single-stranded nucleic acids linked to the antigen binders, each unique combination of two or more UMIs can signify an individual molecule of analyte when detected downstream (e.g. by sequencing or qPCR).

### Definitions

The term “nucleic acid,” as used herein, generally refers to a monomeric or polymeric form of nucleotides of any length, either deoxyribonucleotides or ribonucleotides, or analogs or variants thereof. A nucleic acid molecule may include one or more unmodified or modified nucleotides. Nucleic acid may have any three-dimensional structure, and may perform any function. The following are non-limiting examples of nucleic acids: ribonucleic acid (RNA), deoxyribonucleic acid (DNA), coding or non-coding regions of a gene or gene fragment, loci (locus) defined from linkage analysis, exons, introns, messenger RNA (mRNA), transfer ribonucleic acid (tRNA), ribosomal RNA (rRNA), short interfering RNA (siRNA), short-hairpin RNA (shRNA), micro-RNA (miRNA), ribozymes, complementary deoxyribonucleic acid (cDNA), recombinant polynucleotides, branched polynucleotides, plasmids, vectors, isolated DNA of any sequence, isolated RNA of any sequence, nucleic acid probes, and primers. Nucleic acid may comprise one or more modified nucleotides, such as methylated nucleotides and nucleotide analogs, such as peptide nucleic acid (PNA), Morpholino and locked nucleic acid (LNA), glycol nucleic acid (GNA), threose nucleic acid (TNA), 2′-fluoro, 2′-OMe, and phosphorothiolated DNA. A nucleic acid may include one or more subunits select-ed from adenosine (A), cytosine (C), guanine (G), thymine (T) and uracil (U), or variants thereof. In some examples, a nucleic acid is DNA or RNA, or derivatives thereof. A nucleic acid may be single-stranded or double stranded. A double-stranded nucleic acid may be fully double-stranded or partially double-stranded. A nucleic acid may be a linear nucleic acid. A nucleic acid may be a circular nucleic acid.

As used herein, the term “circular nucleic acid” and grammatical equivalents thereof generally refer to a nucleic acid strand in the form of a closed circle without a free 3′ or 5′ end. A circular nucleic acid may be completely double stranded, completely single stranded, or partially double stranded. A partially double stranded circular nucleic acid may contain one or more (e.g., 2, 3, 4, or more) single stranded regions that separate the same number of double stranded regions.

As used herein, the term “linear nucleic acid” and grammatical equivalents thereof generally refer to a nucleic acid strand in which each (e.g. 3′ or 5′) end is not bound by a covalent bond. A linear nucleic acid molecule can be double stranded, completely single stranded, or partially double stranded. A partially double stranded linear nucleic acid may contain one or more (e.g., 2, 3, 4, or more) single stranded regions that separate the same number of double stranded regions

The term “nucleotide,” as used herein, generally refers to a nucleic acid subunit, which may include A, C, G, T or U, or variants or analogs thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (e.g., A or G, or variant or analogs thereof) or a pyrimidine (e.g., C, T or U, or variant or analogs thereof). A subunit can enable individual nucleic acid bases or groups of bases (e.g., AA, TA, AT, GC, CG, CT, TC, GT, TG, AC, CA, or uracil-counterparts thereof) to be resolved.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens. In some cases, an “antigen” generally refers to an agent comprising an epitope against which an immune response or immunoglobulin is to be generated or is directed. In some cases, an antigen is a molecule which induces an immune reaction.

As used herein, the term “antigen binding moiety” generally refers to a macromolecule that specifically binds to an antigenic determinant. In some cases, an antigen binding moiety comprises or is derived from any antibody, an antigen-binding fragment or derivative of an antibody, or an aptamer.

As used herein, the term “antibody” generally refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as goats, rabbits and mice, as well as non-mammalian species, such as shark immunoglobulins. The term “antibody” generally includes intact immunoglobulins and “antibody fragments” or “antigen binding fragments” that specifically bind to a molecule (or a group of highly similar molecules) to the substantial exclusion of binding to other molecules (for example, antibodies and antibody fragments that have a binding constant for the molecule that is at least 10³ M″¹ greater, at least 10⁴ M″¹ greater or at least 10⁵ M″¹ greater than a binding constant for other molecules in a biological sample). The term “antibody” also generally includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, 111); Kuby, J., Immunology, 3^{rd} Ed., W.H. Freeman & Co., New York, 1997. In some embodiments, “antibody” generally refers to a polypeptide ligand comprising at least a light chain or heavy chain immunoglobulin variable region which specifically recognizes and binds an epitope of an antigen. Antibodies can be composed of a heavy and a light chain, each of which can have a variable region, termed the variable heavy (V_{H}) region and the variable light (V_{L}) region. Together, the V_{H} region and the V_{L} region are responsible for binding the antigen recognized by the antibody. An immunoglobulin (e.g. antibody) can have heavy (H) chains and light (L) chains interconnected by disulfide bonds. There are documented two types of light chain, lambda (λ) and kappa (κ). There are five documented main heavy chain classes (or isotypes) which determine the functional activity of an antibody molecule: IgM, IgD, IgG, IgA and IgE. Each heavy and light chain can contain a constant region and a variable region. In combination, the heavy and the light chain variable regions can specifically bind the antigen. Light and heavy chain variable regions can contain a “framework” region interrupted by three hypervariable regions, also called “complementarity-determining regions” or “CDRs”. The extent of the framework region and CDRs have been documented (see, Kabat et al., Sequences of Proteins of Immunological Interest, U.S. Department of Health and Human Services, 1991, which is hereby incorporated by reference). The sequences of the framework regions of different light or heavy chains can be conserved within a species. The framework region of an antibody, that is the combined framework regions of the constituent light and heavy chains, can largely adopt a β-sheet conformation and the CDRs form loops which connect, and in some cases form part of, the β-sheet structure. Thus, framework regions can act to form a scaffold that provides for positioning the CDRs in correct orientation by inter-chain, non-covalent interactions. The CDRs can be primarily responsible for binding to an epitope of an antigen. The CDRs of each chain can be referred to as CDR1, CDR2, and CDR3, numbered sequentially starting from the N-terminus, and are also identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a specific antigen will have a specific V_{H} region and the V_{L} region sequence, and thus specific CDR sequences. Antibodies with different specificities (e.g. different combining sites for different antigens) can have different CDRs.

The term “antibody” can further encompass digestion fragments, specified portions, derivatives and variants thereof, including antibody mimetics or comprising portions of antibodies that mimic the structure or function of an antibody or specified fragment or portion thereof, including single chain antibodies and fragments thereof. Examples of binding fragments encompassed within the term “antigen binding portion” of an antibody include a Fab fragment, a monovalent fragment comprising the V_{L}, V_{H}, C_{L} and C_{H}, domains; a F(ab′)₂ fragment, a bivalent fragment comprising two Fab fragments linked by a disulfide bridge at the hinge region; a Fd fragment comprising the V_{H} and C_{H}, domains; a Fᵥ fragment comprising the V_{L} and V_{H} domains of a single arm of an antibody, a dAb fragment (Ward et al. (1989) Nature 341:544-546), which comprises a V_{H} domain; and an isolated complementarity determining region (CDR). The two domains of the Fᵥ fragment, V_{L} and V_{H}, can be joined, using recombinant methods, by a synthetic linker that enables them to be made as a single protein chain in which the V_{L} and V_{H} regions pair to form monovalent molecules (known as single chain Fᵥ (scFᵥ)). Bird et al. (1988) Science 242:423-426 and Huston et al. (1988) Proc. Natl. Acad Sci. USA 85:5879-5883). Single chain antibodies can be encompassed within the term “fragment of an antibody.”

“Antibody fragments” or “antigen binding fragments” can include proteolytic antibody fragments (such as F(ab′)₂ fragments, Fab′ fragments, Fab′-SH fragments and Fab fragments as are known in the art), recombinant antibody fragments (such as sFᵥ fragments, dsFᵥ fragments, bispecific sFᵥ fragments, bispecific dsFᵥ fragments, F(ab)′₂ fragments, single chain Fv proteins (“scFᵥ”), disulfide stabilized Fᵥ proteins (“dsFᵥ”), diabodies, and triabodies (as are known in the art), and camelid antibodies (see, for example, U.S. Pat. Nos. 6,015,695; 6,005,079; 5,874,541; 5,840,526; 5,800,988; and 5,759,808). An scFᵥ protein can be a fusion protein in which a light chain variable region of an immunoglobulin and a heavy chain variable region of an immunoglobulin are bound by a linker, while in dsFᵥs, the chains have been mutated to introduce a disulfide bond to stabilize the association of the chains.

As used herein, the term “aptamer” refers to an oligonucleotide that is capable of forming a complex with an intended target substance. Such complex formation is target-specific in the sense that other materials which may accompany the target do not complex to the aptamer. It is recognized that complex formation and affinity are a matter of degree; however, in this context, “target-specific” denotes that the aptamer binds to target with a much higher degree of affinity than it binds to contaminating or “off-target” materials.

The term “barcode,” as used herein, generally refers to a label, or identifier, that can convey or can be capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include barcode sequences, such as: polynucleotide barcodes; random nucleic acid or amino acid sequences; and synthetic nucleic acid or amino acid sequences. A barcode can be attached to an analyte in a reversible or irreversible manner. A barcode can be added to, for example, a fragment of a deoxyribonucleic acid (DNA) or ribonucleic acid (RNA) sample before, during, or after sequencing of the sample. Barcodes can allow for identification or quantification of individual sequencing reads.

As used herein, the term “unique molecular identifier” or “UMI” generally refers to a molecular tag (e.g., a nucleotide sequence) that is attached to a unique DNA or RNA fragment or antibody prior to PCR amplification. After sequencing, a UMI can be used to distinguish sequenced reads from unique DNA or RNA fragments or antibody versus PCR duplicates.

As used herein, the term “hybridization” generally refers to annealing of a complementary sequence to a target nucleic acid (sequence to be detected) by a base pairing interaction. The terms “hybridized” and “hybridize” are generally intended to encompass any specific and reproducible interaction between an oligonucleotide and a target nucleic acid, including binding of regions with partial complementarity and binding interactions that utilize non-canonical interactions to obtain stability or specificity. Nucleotide sequences capable of selective hybridization will generally be at least e.g. 75%, 85%, 90%, 95% 98%, or 100% homologous to the corresponding complementary nucleotide sequence over the length of the oligonucleotide probe. Selectivity can be determined by the salt and temperature conditions during hybridization. For example, a complementary molecule can duplex or hybridize to a corresponding molecule under stringent conditions (e.g., 65° C. and 0.1×SSC {1×SSC=0.15 m nacl, 0.015 m sodium citrate pH 7.0}). In some embodiments, such stringent conditions are those under which the oligonucleotide probe will hybridize to its target sequence but not to other sequences. Stringent conditions are sequence dependent and will be different in different circumstances. Longer sequences can hybridize specifically at higher temperatures. Generally, very stringent conditions can be about 5° C. lower than the thermal melting point (Tm) of the particular sequence at a defined ionic strength and pH. The hybridization temperature is a temperature below the melting temperature (Tm), and the closer the hybridization temperature is to Tm, generally, the more stringent the hybridization, which denotes that mismatched DNA sequences will not hybridize to each other. In some embodiments, the oligonucleotide sequence exceeds genomic DNA to ensure efficient (and quantifiable) hybridization. Stringent conditions can include a salt concentration of at least about 0.01 to 1.0M Na ion concentration (or other salt) at pH 7.0 to 8.3. Stringent conditions can also be achieved by the addition of destabilizing agents such as formamide or tetraalkylammonium salts. Stability of nucleic acid duplexes can be measured by melting temperature or Tm, which generally represents the temperature at which half of the base pairs on average have dissociated between two hybridized molecules.

### EXAMPLE EMBODIMENTS

When the proximity ligation method of FIG. 1A is used to detect multiple distinct protein analytes, and the circular nucleic acid products are to be detected in a same sequencing reaction, an open question is how to ensure that low-abundance and high-abundance protein analytes can be detected with similar accuracy (since the abundance of the circular products will differ). In this case, improved accuracy can be achieved by performing an amplification system (“Norm. PCR”) that results in similar abundance for circular nucleic acid products that result from low- and high-abundance analytes (see FIG. 1F). After normalization, unique combinations of UMIs are used to detect individual abundance of protein analytes; for low-abundant protein analytes each unique UMI combination may be present in multiple copies, whereas for higher-abundance protein analytes the representation of unique UMI combinations approaches a single copy. This can allow for cross-target and cross-sample normalization of DNA concentrations to greatly facilitate the process of library pooling for NGS.

FIG. 1B depicts example organization for three antigen binder-nucleic acid conjugates usable with the detection complex depicted in FIG. 1A.

Ab1, Ab2, and Ab3 signify three antigen-binders configured to form the structure depicted in FIG. 1A, which are all directed against a same protein analyte. These antigen-binders can comprise antigen binders (e.g. full-length IgG molecules), fragments of antigen binders (e.g. Fab fragments), derivatives of antigen binders (e.g. scFvs), or aptamers. If the antigen-binders are antibodies, the antigen binders can be monoclonal or polyclonal in derivation.

Ab1, Ab2, and Ab3 are each conjugated to a proximal nucleic acid that can comprise (a) L₁, L₂, and L₃; (b) A₁, A₂, and A₃; (c) U₁, U₂, and U₃; or (d) T₁, T₂, and T₃.

L₁, L₂, and L₃ signify regions of nucleic acid (or a non-nucleic acid molecule) used as a linker to distance the antigen binders from the tripartite conjugate depicted in FIG. 1A. In some cases, nucleic acid regions can comprise from one up to 40-60 nucleotides of a polyadenine sequence or a polythymidine sequence. In some cases, a non-nucleic acid molecule can comprise a polymeric linker such as polyethylene glycol. In some cases, the linkers are configured to control the effective concentration of the individual reporter molecule components such that they associate when all the antigen binder-nucleic acid conjugates bind a single target.

A₁, A₂, and A₃ signify regions of nucleic acid (e.g. DNA) that comprise at least a unique nucleic acid sequence. In some cases, the antigen binder-nucleic acid conjugates are envisioned for use in a multiplexed pool where there are multiple sets of coincident antigen binder-nucleic acid conjugates, wherein each coincident antigen binder-nucleic acid conjugates set is directed against a different protein analyte. In this case, A₁, A₂, and A₃ encode a sequence that uniquely identifies the protein target that the antigen binder-nucleic acid conjugate set is directed against. In some cases, A₁ is up to 40-50 nucleotides in length. In some cases, A₁ comprises one or more of: a reverse primer sequence, a forward primer sequence, or a restriction enzyme cut site (e.g. an EcoRV cut site), or a reverse complement of any of these. In some cases, the forward and reverse primers comprise sequences complementary to that of Illumina adapter primers. In some cases, A₂ and A₃ are shorter sequence of up to 20-30 nucleotides that also uniquely identify the protein target they are directed against.

U₁, U₂, and U₃ signify regions of nucleic acid comprising unique molecular identifiers (UMIs) that differ for each individual molecule of antigen binder conjugated to the nucleic acid. When detected via sequencing of a molecule such as that depicted in FIG. 1A, each unique combination of U₁, U₂, and U₃ can signify an individual binding event and thus an individual molecule of protein analyte in a protein analyte-containing sample. In some cases U₁, U₂, and U₃ comprise up to 12 nucleotides in length. In some cases, U₁, U₂, and U₃ are optional.

T₁, T₂, and T₃ signify adapter (or “toehold”) sequences that allow formation of the tripartite structure depicted in FIG. 1A via hybridization. T₁, T₂, and T₃ are configured to hybridize to corresponding sequences T₁′, T₂′, and T₃′ which are on distal nucleic acid molecules associated with separate antigen binder-nucleic acid conjugates to form the reporter nucleic acid structure in FIG. 1A, and can be up to 4-15 nucleotides in length. In some cases, the affinity of binding for the toehold regions can be optimized to control the relative output of different analyte nucleic acid reporters.

Ab1, Ab2, and Ab3 are each associated with a distal nucleic acid, which is associated with the proximal nucleic acid via presence of a corresponding A₁′, A₂′, and A₃′ region in the distal nucleic acid. A₁′, A₂′, and A₃′ signify nucleic acid regions configured to hybridize to A₁, A₂, and A₃. In some cases they are partially or completely complementary to A₁, A₂, and A₃. The distal nucleic acid can comprise (a) A₁′, A₂′, and A₃′; (b) I₁, I₂, and I₃; and (c) T₁′, T₂′, and T₃′. In some cases, the distal nucleic acid can also comprise one or more unique molecular identifier (UMI) sequences.

I₁, I₂, and b signify indexing sequences, which can be sample specific indexes. In some cases, I₁ can signify a row number, I₂ can signify a column number, and I₃ can signify a plate number when referring to a multiwell plate of samples containing protein analytes. In some cases, these sequences I₁, I₂, and I₃ can comprise up to 5-10 nucleotides in length.

In some cases, to amplify the signal output by the tripartite binding event, Ab1, Ab2, and Ab3 are each conjugated to more than one proximal nucleic acid that can comprise additional unique antigen reporter barcodes or UMIs (see e.g. FIG. 1BA).

FIG. 1C depicts a three-component circular nucleic acid product which can signify a binding event, which can be produced by hybridization, optional gap filling, and ligation of the structure depicted in FIG. 1A (optionally T4 polynucleotide kinase treatment if phosphorylated molecules are not provided attached to the antigen binders).

A₁′, A₂′, and A₃′ are described as in FIG. 1A (e.g. as Ab₁′, Ab₂′, and Ab₃′), as are I₁, I₂, and I₃ and T₁′, T₂′, and T₃′. U₁′, U₂′, and U₃′ signify complements of U₁, U₂, and U₃ produced by optional gap filling of the structure depicted in FIG. 1A. As described in FIG. 1B, each unique combination of UMIs U₁, U₂, and U₃ can signify an individual binding event and thus an individual molecule of protein analyte in a protein analyte-containing sample. Thus, sequencing of the circular nucleic acid products produced or sequencing of products derived from the circular nucleic acid products produced can provide a route to identify the total number of protein analytes in the sample (e.g. by identifying the number of unique sequence U₁, U₂, and U₃ products) and each sample can be distinguished by the unique combination of I1, I2, I3. In FIG. 1C depicted, A₁′ comprises a reverse primer sequence and a forward primer sequence, complementary to that of Illumina adapter primers (Ad1 and Ad2). In some embodiments, Ad1 and Ad2 can be located in any portion of the product generated by the methods disclosed herein.

In one analysis method for FIG. 1C, PCR amplification of the circular nucleic acid product of FIG. 1C with adapter primers Ad1 and Ad2 allows for the production of linear nucleic acid molecules as in FIG. 1D. In some cases, the PCR amplification is optimized so that a multiplexed reaction produces about 10 nM total linear DNA.

In a second analysis method for FIG. 1C, A₁′ (or alternatively A₂′ or A₃′) comprises a restriction enzyme site (e.g. an EcoRV cut site), another disruptable site, or polymerase blocking site (e.g., polyA, UV-cleavable linker, or PEG linker) and a reverse primer sequence and a forward primer sequence. In this analysis method, digestion with the restriction enzyme linearizes the circular products according to FIG. 1C and the products are subjected to a bead-based normalization protocol (see FIG. 1E).

When the proximity ligation method of FIG. 1A is used to detect multiple distinct protein analytes, and the circular nucleic acid products are to be detected in a same sequencing reaction, an open question is how to ensure that low-abundance and high-abundance protein analytes can be detected with similar accuracy (since the abundance of the circular products will differ). In this case, improved accuracy can be achieved by performing an amplification system? (“Norm. PCR”) that results in similar abundance for circular nucleic acid products that result from low- and high-abundance analytes (see FIG. 1F). After normalization, unique combinations of UMIs are used to detect individual abundance of protein analytes; for low-abundant protein analytes each unique UMI combination may be present in multiple copies, whereas for higher-abundance protein analytes the representation of unique UMI combinations approaches a single copy. This can allow for cross-target and cross-sample normalization of DNA concentrations to greatly facilitate the process of library pooling for NGS.

One scheme for such a normalization amplification procedure is depicted in FIG. 1F, in which bridge amplification on a defined number of beads is used to ensure production of equal amounts of circular products for each protein analyte (such a procedure can also be used to normalize other nucleic acid samples comprising indexes). This scheme begins with products of the type depicted in FIG. 1E, where A₁′ (or alternatively A₂′ or A₃′) comprises a restriction enzyme site (e.g. an EcoRV cut site) and a reverse primer sequence and a forward primer sequence; forward and reverse primer sequence are individual to each protein analyte in a multiplex reaction. Linearized products as depicted in FIG. 1E are incubated with a population of beads that comprises subpopulations of equal number directed against circular nucleic acids representative of each protein analyte. Each subpopulation of beads in turn comprises equal loadings of forward and reverse primers specific for circular nucleic acids representative of each protein analyte. Equal loading of the forward/reverse primers can be achieved by a suitable chemical conjugation method (e.g. biotin/streptavidin attachment) of the primers to the beads (where defined loading of biotin or streptavidin or another conjugation moiety on the beads is initially provided). Successive annealing (“step 1”), followed by extension (“step 2”) and exonuclease treatment provides a growing population of amplified products on the beads (“step 3”). Repetition of this process to saturation of the beads, followed by exonuclease treatment and purification of the beads, provides a defined population of amplified products determined by the forward/reverse primer loading on the beads. Removal of the amplified products (or PCR amplification of the products) from the beads provides linear products that can be sequenced by next generation sequencing. This protocol to FIG. 1E can also be used with circular products.

FIG. 1G depicts an alternative detection intermediate to FIG. 1A, in which two antigen binder-nucleic acid conjugates (Ab5 and Ab6) conjugated to single-stranded barcode-bearing nucleic acids are provided, and one antigen binder (Ab4)—is provided attached to a bead (the bead in turn being conjugated to two separate single-stranded barcode-bearing nucleic acids). In this configuration, both the nucleic acids conjugated to antibodies (T₅′, T₆, T₆′, T₄′) and the nucleic acids conjugated to the bead (T₅, T₄) comprise toehold regions that are configured that so when Ab4/Ab5/Ab6 bind a single common analyte, production of a loop double-stranded can be formed given alternating polymerase, denaturation, and polymerase operations. Cleavage of the loop double-stranded nucleic acid product from the beads via a suitable method enables detection of the binding event (e.g. by next-generation sequencing).

FIGS. 21, 22, 23, 24, 25, and 26 depict alternate organizations of intermediates that can be used to detect antigens using antigen binders according to methods of the disclosure. FIG. 21 depicts embodiments using partially double-stranded nucleic acid-conjugated antigen binders to produce circular detectable nucleic acids using 2 (“2Ab”), 3 (“3Ab”), or 4 (“4Ab”) antigen binders. FIG. 22 depicts embodiments using at least two tunable nucleotides according to the disclosure and 2 (“2Ab”), 3 (“3Ab”), or 4 (“4Ab”) partially double-stranded nucleic acid-conjugated antigen binders to produce circular detectable nucleic acids. FIG. 23 depicts embodiments using one tunable nucleotide according to the disclosure and 2 (“2Ab”) or 3 (“3Ab”) partially double-stranded nucleic acid-conjugated antigen binders to produce a circular detectable nucleic acid. FIG. 24 depicts embodiments where 2 template oligonucleotides according to the disclosure and 2 partially double-stranded nucleic acid-conjugated antigen binders not configured to hybridize to each other (top panel) or 3 template oligonucleotides according to the disclosure and 3 partially double-stranded nucleic acid-conjugated antigen binders not configured to hybridize to one another are used to produce circular detectable nucleic acids. FIG. 25 depicts embodiments using partially double-stranded nucleic acid-conjugated antigen binders to produce linear detectable nucleic acids using 2 (“2Ab”), 3 (“3Ab”), or 4 (“4Ab”) antigen binders. FIG. 26 depicts embodiments using a single tunable nucleotide according to the disclosure and 2 (“2Ab”), 3 (“3Ab”), or 4 (“4Ab”) partially double-stranded nucleic acid-conjugated antigen binders to produce linear detectable nucleic acids.

### Sample Types

A sample as described herein (e.g. containing one or more antigens to be detected according to methods described herein) may be from a subject, such as a patient. A sample may be an environmental sample. A sample may comprise food. A sample can comprise a pathogen antigen, a human antigen, an environmental contaminant, a tumor antigen, or any combination thereof. Methods for detecting molecules (e.g., nucleic acids, proteins, etc.) in a subject in order to detect, diagnose, monitor, predict, or evaluate the status or outcome of a condition are described in this disclosure. In some cases, the molecules are circulating molecules (e.g., unbound to cells and freely circulating in bodily fluids such as blood, blood plasma or blood serum). In some cases, the molecules are expressed in the cytoplasm of blood, endothelial, or organ cells. In some cases, the molecules are expressed on the surface of blood, endothelial, or organ cells. In some embodiments, the sample is cell-free. In some embodiments, the environmental contaminant is present in the patient sample.

The methods, kits, and systems disclosed herein can be used to classify one or more samples from one or more subjects. A sample can comprise any material containing tissues, cells, nucleic acids, genes, gene fragments, expression products, proteins, polypeptides, exosomes, gene expression products, or gene expression product fragments of a subject to be tested. A sample can include but is not limited to, tissue, cells, plasma, serum, or any other biological material from cells or derived from cells of an individual. The sample can be a heterogeneous or homogeneous population of cells or tissues. The sample can be a fluid that is acellular or depleted of cells (e.g., plasma or serum). In some cases, the sample is from a single patient. In some cases, the method comprises analyzing multiple samples at once, e.g., via massively parallel multiplex analysis on protein arrays or the like.

The sample may be a bodily fluid. The bodily fluid can be saliva, urine, or blood. The sample can be a fraction of any of these fluids, such as plasma, serum or exosomes. In preferred embodiments, the sample is a blood sample, plasma sample, or serum sample. A subject can be a healthy individual, an individual that has or is suspected of having a disease or a predisposition to the disease, or an individual in need of therapy or suspected of needing therapy. The terms “individual” or “patient” are intended to be interchangeable with “subject.”

In an example embodiment, the sample is derived from a human. In an alternative embodiment, the sample is from an environment. Non-limiting examples of environmental samples include food, water, soil, slurries, debris, biofilms, samples from containers of aqueous fluids, airborne particles or aerosols and the like waste, or air.

### Antigen Binders

An antigen binder as described herein can comprise any molecule capable of binding to an antigen and reporting said binding by virtue of a nucleic acid. In some cases, an antigen binder comprises an antigen-binding moiety which can include an antibody, an antigen-binding fragment or derivative of an antibody, or a nucleic acid aptamer (e.g. an oligonucleotide that binds an antigen).

An antibody may be monoclonal or polyclonal. Further, the antibodies may be full length, or an antigen binding fragment or derivative, such as a F(ab′)2, Fab′, Fab, Fv, sFv, scFv, or a hybrid fragments thereof. In some embodiments, an antigen binding derivative comprises conjugates of antibody fragments and antigen binding proteins (single chain antibodies). In some embodiments, any antibody comprises immunoglobulin single variable domains, such as in the case of a nanobody. The antibodies may also be naturally occurring antibodies, humanized antibodies or chimeric antibodies. In some embodiments, an antibody is specific for one antigen. In these embodiments, the antibody selectively binds that one antigen and no other antigens. An antibody may be a polyclonal antibody or a monoclonal antibody. In some embodiments, an antibody is a fragment or polymer of an antibody.

An antibody can include proteins having the characteristic two-armed, Y-shape of an antibody molecule as well as one or more fragments of an antibody that retain the ability to specifically bind to an antigen. Example antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CH1 domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for an antibody, wherein the antibody is a Fv antibody, including Fv antibodies comprised of the minimum antibody fragment which contains a complete antigen-recognition and antigen-binding site.

In some embodiments, the Fv antibody comprises a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for an antibody, wherein the antibody is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

In some embodiments, an antibody can be a monoclonal antibody. A monoclonal antibody as used herein refers to an antibody obtained from a substantially homogeneous population of antibodies, e.g., the individual antibodies within the population are identical except for possible naturally occurring mutations that may be present in a small subset of the antibody molecules. The monoclonal antibodies herein may include “chimeric” antibodies in which a portion of the heavy or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit antagonistic activity.

In some embodiments, an antibody may be an antibody or antigen binding fragment. The fragment may include chimeric antibodies and hybrid antibodies, with dual or multiple antigen or epitope specificities, and fragments, such as

### Linked Nucleic Acids

In some cases, antigen binders as described herein further comprise an antigen-binding moiety and a nucleic acid linked thereto by a covalent or non-covalent linkage to serve as moiety for reporting their binding to an antigen. In some cases, a covalent linkage is provided to the nucleic acid via amino-end (either 3′ or 5′) modification of the nucleic acid (e.g. 5-Amino-Modifier C12. from IDT), followed by conversion to 4-formylbenzamide groups with succinimidyl 4-formylbenzoate (S-4B); complementary derivatization of a peptidic antigen-binding moiety with succinimidyl 6-hydrainonicotinate acetone hydrazone (SANH) to introduce corresponding aromatic hydrazine molecules to the peptidic antigen moiety allows for the two molecules to be reacted to form a stable conjugate. In some embodiments, the reactivity class comprises a carbonyl, thiol, amine, carboxyl-to-amine, azide, aldehyde, photo, or carbohydrate reactive group. In some embodiments, the reactive chemical group comprises N-hydroxysuccinimide esters (NHS esters) compound. In some embodiments, the reactive chemical group comprises a maleimide compound. The reactive chemical group can comprise an NHS ester, imidoester, pentafluorophenyl ester, diazirine, aryl azide, hydroxymethyl phosphine, carbodiimide, haloacetyl, pyridyl disulfide, thiosulfonate, vinyl sulfone, hydrazide, alkoxyamine, alkyne, or phosphine. In some embodiments, a crosslinker is used to provide a covalent or non-covalent linkage. A crosslinker can be disuccinimidyl suberate (DSS). sulfosuccinimidyl 4-(N-maleimidomethyl)cyclohexane-1-carboxylate (Sulfo-SMCC), sulfo-SBED, bis(sulfosuccinimidyl) suberate, or bis(succinimidyl) penta(ethylene glycol).

### Double-Stranded Nucleic Acid

In some embodiments, the antigen binder comprises a double-stranded nucleic acid. In some embodiments, the double-stranded nucleic acid is directly attached to an antibody. In some embodiments, the double-stranded nucleic acid is attached to a solid surface to which an antibody or other antigen-binding moiety is also attached. In some embodiments, the solid surface is a bead.

In some embodiments, the double-stranded nucleic acid is partially double-stranded or fully double-stranded. In some embodiments, a partially double-stranded nucleic acid is generated by two single-stranded nucleic acids which hybridize to generate a single stranded region and a double-stranded region. In some embodiments, fully double-stranded nucleic acid is generated by two single-stranded nucleic acids which hybridize to form a double-stranded nucleic acid without single-stranded regions. In some embodiments, the double-stranded nucleic acid forms a loop double-stranded nucleic acid.

In some embodiments, the partially double-stranded nucleic acid is double-stranded at the 5′-end. In some embodiments, the partially double-stranded nucleic acid is double stranded at the 3′-end. In some embodiments, the partially double-stranded nucleic acid is single-stranded at the 5′-end. In some embodiments, the partially double-stranded nucleic acid is single stranded at the 3′-end. In some embodiments, the partially double-stranded nucleic acid is double-stranded on the 3′- and 5′-ends. In these embodiments, the partially double-stranded nucleic acid is single-stranded in the center of the partially double-stranded nucleic acid. In some embodiments, the partially double-stranded nucleic acid is single-stranded on the 3′- and 5′-ends. In these embodiments, the partially double-stranded nucleic acid is double-stranded in the center of the partially double-stranded nucleic acid.

In some embodiments, the partially double-stranded nucleic acid comprises a sequence for a barcode, a sample index, a unique molecular identifier (UMI), a hybridization region, a primer, a sequencing adapter, an endonuclease site, or any combination thereof. In some embodiments, the partially double-stranded nucleic acid may be specific to an antigen. In some embodiments, the partially double-stranded nucleic acid may be specific to an antigen binder. In some embodiments, the hybridization region of the partially double-stranded nucleic acid specific to an antigen. In some embodiments, the hybridization region of the partially double-stranded nucleic acid specific to an antigen binder. In some embodiments, the hybridization region of the partially double-stranded nucleic acid hybridizes to a hybridization region on an antigen. In some embodiments, the hybridization region of the partially double-stranded nucleic acid hybridizes to a hybridization region on an antigen binder. A hybridization region between the A oligonucleotide and the O oligonucleotide can be about 12 nucleic acids long. A hybridization region between the A oligonucleotide and the O oligonucleotide can be about 13 nucleic acids long. A hybridization region between the A oligonucleotide and the O oligonucleotide can be about 14 nucleic acids long. A hybridization region between the A oligonucleotide and the O oligonucleotide can be about 15 nucleic acids long. A hybridization region between the A oligonucleotide and the O oligonucleotide can be about 16 nucleic acids long. A hybridization region between the A oligonucleotide and the O oligonucleotide can be about 17 nucleic acids long. A hybridization region between the A oligonucleotide and the O oligonucleotide can be about 18 nucleic acids long. A hybridization region between the A oligonucleotide and the O oligonucleotide can be about 19 nucleic acids long. A hybridization region between the A oligonucleotide and the O oligonucleotide can be about 20 nucleic acids long. A hybridization region between the A oligonucleotide and the O oligonucleotide can be about 21 nucleic acids long. A hybridization region between the A oligonucleotide and the O oligonucleotide can be about 22 nucleic acids long. A hybridization region between the A oligonucleotide and the O oligonucleotide can be about 23 nucleic acids long. A hybridization region between the A oligonucleotide and the O oligonucleotide can be about 24 nucleic acids long. A hybridization region between the A oligonucleotide and the O oligonucleotide can be about 25 nucleic acids long. A hybridization region between the A oligonucleotide and the O oligonucleotide can be about 26 nucleic acids long. A hybridization region between the A oligonucleotide and the O oligonucleotide can be about 27 nucleic acids long. A hybridization region between the A oligonucleotide and the O oligonucleotide can be about 28 nucleic acids long. A hybridization region between the A oligonucleotide and the O oligonucleotide can be about 29 nucleic acids long. A hybridization region between the A oligonucleotide and the O oligonucleotide can be about 30 nucleic acids long. A hybridization region between the A oligonucleotide and the O oligonucleotide can be about 31 nucleic acids long. A hybridization region between the A oligonucleotide and the O oligonucleotide can be about 32 nucleic acids long. A hybridization region between the A oligonucleotide and the O oligonucleotide can be about 33 nucleic acids long. A hybridization region between the A oligonucleotide and the O oligonucleotide can be about 34 nucleic acids long. A hybridization region between the A oligonucleotide and the O oligonucleotide can be about 35 nucleic acids long. A hybridization region between the A oligonucleotide and the O oligonucleotide can be about 36 nucleic acids long. A hybridization region between the A oligonucleotide and the O oligonucleotide can be about 37 nucleic acids long. A hybridization region between the A oligonucleotide and the O oligonucleotide can be about 38 nucleic acids long. A hybridization region between the A oligonucleotide and the O oligonucleotide can be about 39 nucleic acids long. A hybridization region between the A oligonucleotide and the O oligonucleotide can be about 40 nucleic acids long. A hybridization region between the A oligonucleotide and the O oligonucleotide can be about 41 nucleic acids long. A hybridization region between the A oligonucleotide and the O oligonucleotide can be about 42 nucleic acids long. A hybridization region between the A oligonucleotide and the O oligonucleotide can be about 43 nucleic acids long. A hybridization region between the A oligonucleotide and the O oligonucleotide can be about 44 nucleic acids long. A hybridization region between the A oligonucleotide and the O oligonucleotide can be about 45 nucleic acids long. A hybridization region between the A oligonucleotide and the O oligonucleotide can be about 46 nucleic acids long. A hybridization region between the A oligonucleotide and the O oligonucleotide can be about 47 nucleic acids long. A hybridization region between the A oligonucleotide and the O oligonucleotide can be about 48 nucleic acids long. A hybridization region between the A oligonucleotide and the O oligonucleotide can be about 49 nucleic acids long. A hybridization region between the A oligonucleotide and the O oligonucleotide can be about 50 nucleic acids long. A hybridization region between the A oligonucleotide and the O oligonucleotide can be about 51 nucleic acids long. A hybridization region between the A oligonucleotide and the O oligonucleotide can be about 52 nucleic acids long. A hybridization region between the A oligonucleotide and the O oligonucleotide can be about 53 nucleic acids long. A hybridization region between the A oligonucleotide and the O oligonucleotide can be about 54 nucleic acids long. A hybridization region between the A oligonucleotide and the O oligonucleotide can be about 55 nucleic acids long. A hybridization region between the A oligonucleotide and the O oligonucleotide can be about 56 nucleic acids long. A hybridization region between the A oligonucleotide and the O oligonucleotide can be about 57 nucleic acids long. A hybridization region between the A oligonucleotide and the O oligonucleotide can be about 58 nucleic acids long. A hybridization region between the A oligonucleotide and the O oligonucleotide can be about 59 nucleic acids long. A hybridization region between the A oligonucleotide and the O oligonucleotide can be about 60 nucleic acids long. A hybridization region between the A oligonucleotide and the O oligonucleotide can be about 61 nucleic acids long. A hybridization region between the A oligonucleotide and the O oligonucleotide can be about 62 nucleic acids long. A hybridization region between the A oligonucleotide and the O oligonucleotide can be about 63 nucleic acids long. A hybridization region between the A oligonucleotide and the O oligonucleotide can be about 64 nucleic acids long. A hybridization region between the A oligonucleotide and the O oligonucleotide can be about 65 nucleic acids long. A hybridization region between the A oligonucleotide and the O oligonucleotide can be about 66 nucleic acids long. A hybridization region between the A oligonucleotide and the O oligonucleotide can be about 67 nucleic acids long. A hybridization region between the A oligonucleotide and the O oligonucleotide can be about 68 nucleic acids long. A hybridization region between the A oligonucleotide and the O oligonucleotide can be about 69 nucleic acids long. A hybridization region between the A oligonucleotide and the O oligonucleotide can be about 70 nucleic acids long. A hybridization region between the A oligonucleotide and the O oligonucleotide can be about 71 nucleic acids long. A hybridization region between the A oligonucleotide and the O oligonucleotide can be about 72 nucleic acids long. A hybridization region between the A oligonucleotide and the O oligonucleotide can be about 73 nucleic acids long. A hybridization region between the A oligonucleotide and the O oligonucleotide can be about 74 nucleic acids long. A hybridization region between the A oligonucleotide and the O oligonucleotide can be about 75 nucleic acids long. A hybridization region between the A oligonucleotide and the O oligonucleotide can be about 76 nucleic acids long. A hybridization region between the A oligonucleotide and the O oligonucleotide can be about 77 nucleic acids long. A hybridization region between the A oligonucleotide and the O oligonucleotide can be about 78 nucleic acids long. A hybridization region between the A oligonucleotide and the O oligonucleotide can be about 79 nucleic acids long. A hybridization region between the A oligonucleotide and the O oligonucleotide can be about 80 nucleic acids long. A hybridization region between the A oligonucleotide and the O oligonucleotide can be about 12 to about 80 nucleic acids long. A hybridization region between the A oligonucleotide and the O oligonucleotide can be about 15 to about 75 nucleic acids long. A hybridization region between the A oligonucleotide and the O oligonucleotide can be about 20 to about 70 nucleic acids long. A hybridization region between the A oligonucleotide and the O oligonucleotide can be about 25 to about 65 nucleic acids long. A hybridization region between the A oligonucleotide and the O oligonucleotide can be about 30 to about 60 nucleic acids long. A hybridization region between the A oligonucleotide and the O oligonucleotide can be about 35 to about 55 nucleic acids long. A hybridization region between the A oligonucleotide and the O oligonucleotide can be about 40 to about 50 nucleic acids long.

The single stranded region on the A oligonucleotide can be 5 nucleic acids long to 70 nucleic acids long. The single stranded region on the A oligonucleotide can be about 5 nucleic acids long. The single stranded region on the A oligonucleotide can be about 6 nucleic acids long. The single stranded region on the A oligonucleotide can be about 7 nucleic acids long. The single stranded region on the A oligonucleotide can be about 8 nucleic acids long. The single stranded region on the A oligonucleotide can be about 9 nucleic acids long. The single stranded region on the A oligonucleotide can be about 10 nucleic acids long. The single stranded region on the A oligonucleotide can be about 11 nucleic acids long. The single stranded region on the A oligonucleotide can be about 12 nucleic acids long. The single stranded region on the A oligonucleotide can be about 13 nucleic acids long. The single stranded region on the A oligonucleotide can be about 14 nucleic acids long. The single stranded region on the A oligonucleotide can be about 15 nucleic acids long. The single stranded region on the A oligonucleotide can be about 16 nucleic acids long. The single stranded region on the A oligonucleotide can be about 17 nucleic acids long. The single stranded region on the A oligonucleotide can be about 18 nucleic acids long. The single stranded region on the A oligonucleotide can be about 19 nucleic acids long. The single stranded region on the A oligonucleotide can be about 20 nucleic acids long. The single stranded region on the A oligonucleotide can be about 21 nucleic acids long. The single stranded region on the A oligonucleotide can be about 22 nucleic acids long. The single stranded region on the A oligonucleotide can be about 23 nucleic acids long. The single stranded region on the A oligonucleotide can be about 24 nucleic acids long. The single stranded region on the A oligonucleotide can be about 25 nucleic acids long. The single stranded region on the A oligonucleotide can be about 26 nucleic acids long. The single stranded region on the A oligonucleotide can be about 27 nucleic acids long. The single stranded region on the A oligonucleotide can be about 28 nucleic acids long. The single stranded region on the A oligonucleotide can be about 29 nucleic acids long. The single stranded region on the A oligonucleotide can be about 30 nucleic acids long. The single stranded region on the A oligonucleotide can be about 31 nucleic acids long. The single stranded region on the A oligonucleotide can be about 32 nucleic acids long. The single stranded region on the A oligonucleotide can be about 33 nucleic acids long. The single stranded region on the A oligonucleotide can be about 34 nucleic acids long. The single stranded region on the A oligonucleotide can be about 35 nucleic acids long. The single stranded region on the A oligonucleotide can be about 36 nucleic acids long. The single stranded region on the A oligonucleotide can be about 37 nucleic acids long. The single stranded region on the A oligonucleotide can be about 38 nucleic acids long. The single stranded region on the A oligonucleotide can be about 39 nucleic acids long. The single stranded region on the A oligonucleotide can be about 40 nucleic acids long. The single stranded region on the A oligonucleotide can be about 41 nucleic acids long. The single stranded region on the A oligonucleotide can be about 42 nucleic acids long. The single stranded region on the A oligonucleotide can be about 43 nucleic acids long. The single stranded region on the A oligonucleotide can be about 44 nucleic acids long. The single stranded region on the A oligonucleotide can be about 45 nucleic acids long. The single stranded region on the A oligonucleotide can be about 46 nucleic acids long. The single stranded region on the A oligonucleotide can be about 47 nucleic acids long. The single stranded region on the A oligonucleotide can be about 48 nucleic acids long. The single stranded region on the A oligonucleotide can be about 49 nucleic acids long. The single stranded region on the A oligonucleotide can be about 50 nucleic acids long. The single stranded region on the A oligonucleotide can be about 51 nucleic acids long. The single stranded region on the A oligonucleotide can be about 52 nucleic acids long. The single stranded region on the A oligonucleotide can be about 53 nucleic acids long. The single stranded region on the A oligonucleotide can be about 54 nucleic acids long. The single stranded region on the A oligonucleotide can be about 55 nucleic acids long. The single stranded region on the A oligonucleotide can be about 56 nucleic acids long. The single stranded region on the A oligonucleotide can be about 57 nucleic acids long. The single stranded region on the A oligonucleotide can be about 58 nucleic acids long. The single stranded region on the A oligonucleotide can be about 59 nucleic acids long. The single stranded region on the A oligonucleotide can be about 60 nucleic acids long. The single stranded region on the A oligonucleotide can be about 61 nucleic acids long. The single stranded region on the A oligonucleotide can be about 62 nucleic acids long. The single stranded region on the A oligonucleotide can be about 63 nucleic acids long. The single stranded region on the A oligonucleotide can be about 64 nucleic acids long. The single stranded region on the A oligonucleotide can be about 65 nucleic acids long. The single stranded region on the A oligonucleotide can be about 66 nucleic acids long. The single stranded region on the A oligonucleotide can be about 67 nucleic acids long. The single stranded region on the A oligonucleotide can be about 68 nucleic acids long. The single stranded region on the A oligonucleotide can be about 69 nucleic acids long. The single stranded region on the A oligonucleotide can be about 70 nucleic acids long. The single stranded region on the A oligonucleotide can be about 5 to about 70 nucleic acids long. The single stranded region on the A oligonucleotide can be about 10 to about 65 nucleic acids long. The single stranded region on the A oligonucleotide can be about 15 to about 60 nucleic acids long. The single stranded region on the A oligonucleotide can be about 20 to about 55 nucleic acids long. The single stranded region on the A oligonucleotide can be about 25 to about 50 nucleic acids long. The single stranded region on the A oligonucleotide can be about 30 to about 45 nucleic acids long. The single stranded region on the A oligonucleotide can be about 35 to about 40 nucleic acids long.

The single stranded region on the O oligonucleotide can be 5 nucleic acids long to 180 nucleic acids long. The single stranded region on the O oligonucleotide can be about 5 nucleic acids long. The single stranded region on the O oligonucleotide can be about 6 nucleic acids long. The single stranded region on the O oligonucleotide can be about 7 nucleic acids long. The single stranded region on the O oligonucleotide can be about 8 nucleic acids long. The single stranded region on the O oligonucleotide can be about 9 nucleic acids long. The single stranded region on the O oligonucleotide can be about 10 nucleic acids long. The single stranded region on the O oligonucleotide can be about 11 nucleic acids long. The single stranded region on the O oligonucleotide can be about 12 nucleic acids long. The single stranded region on the O oligonucleotide can be about 13 nucleic acids long. The single stranded region on the O oligonucleotide can be about 14 nucleic acids long. The single stranded region on the O oligonucleotide can be about 15 nucleic acids long. The single stranded region on the O oligonucleotide can be about 16 nucleic acids long. The single stranded region on the O oligonucleotide can be about 17 nucleic acids long. The single stranded region on the O oligonucleotide can be about 18 nucleic acids long. The single stranded region on the O oligonucleotide can be about 19 nucleic acids long. The single stranded region on the O oligonucleotide can be about 20 nucleic acids long. The single stranded region on the O oligonucleotide can be about 21 nucleic acids long. The single stranded region on the O oligonucleotide can be about 22 nucleic acids long. The single stranded region on the O oligonucleotide can be about 23 nucleic acids long. The single stranded region on the O oligonucleotide can be about 24 nucleic acids long. The single stranded region on the O oligonucleotide can be about 25 nucleic acids long. The single stranded region on the O oligonucleotide can be about 26 nucleic acids long. The single stranded region on the O oligonucleotide can be about 27 nucleic acids long. The single stranded region on the O oligonucleotide can be about 28 nucleic acids long. The single stranded region on the O oligonucleotide can be about 29 nucleic acids long. The single stranded region on the O oligonucleotide can be about 30 nucleic acids long. The single stranded region on the O oligonucleotide can be about 31 nucleic acids long. The single stranded region on the O oligonucleotide can be about 32 nucleic acids long. The single stranded region on the O oligonucleotide can be about 33 nucleic acids long. The single stranded region on the O oligonucleotide can be about 34 nucleic acids long. The single stranded region on the O oligonucleotide can be about 35 nucleic acids long. The single stranded region on the O oligonucleotide can be about 36 nucleic acids long. The single stranded region on the O oligonucleotide can be about 37 nucleic acids long. The single stranded region on the O oligonucleotide can be about 38 nucleic acids long. The single stranded region on the O oligonucleotide can be about 39 nucleic acids long. The single stranded region on the O oligonucleotide can be about 40 nucleic acids long. The single stranded region on the O oligonucleotide can be about 41 nucleic acids long. The single stranded region on the O oligonucleotide can be about 42 nucleic acids long. The single stranded region on the O oligonucleotide can be about 43 nucleic acids long. The single stranded region on the O oligonucleotide can be about 44 nucleic acids long. The single stranded region on the O oligonucleotide can be about 45 nucleic acids long. The single stranded region on the O oligonucleotide can be about 46 nucleic acids long. The single stranded region on the O oligonucleotide can be about 47 nucleic acids long. The single stranded region on the O oligonucleotide can be about 48 nucleic acids long. The single stranded region on the O oligonucleotide can be about 49 nucleic acids long. The single stranded region on the O oligonucleotide can be about 50 nucleic acids long. The single stranded region on the O oligonucleotide can be about 51 nucleic acids long. The single stranded region on the O oligonucleotide can be about 52 nucleic acids long. The single stranded region on the O oligonucleotide can be about 53 nucleic acids long. The single stranded region on the O oligonucleotide can be about 54 nucleic acids long. The single stranded region on the O oligonucleotide can be about 55 nucleic acids long. The single stranded region on the O oligonucleotide can be about 56 nucleic acids long. The single stranded region on the O oligonucleotide can be about 57 nucleic acids long. The single stranded region on the O oligonucleotide can be about 58 nucleic acids long. The single stranded region on the O oligonucleotide can be about 59 nucleic acids long. The single stranded region on the O oligonucleotide can be about 60 nucleic acids long. The single stranded region on the O oligonucleotide can be about 61 nucleic acids long. The single stranded region on the O oligonucleotide can be about 62 nucleic acids long. The single stranded region on the O oligonucleotide can be about 63 nucleic acids long. The single stranded region on the O oligonucleotide can be about 64 nucleic acids long. The single stranded region on the O oligonucleotide can be about 65 nucleic acids long. The single stranded region on the O oligonucleotide can be about 66 nucleic acids long. The single stranded region on the O oligonucleotide can be about 67 nucleic acids long. The single stranded region on the O oligonucleotide can be about 68 nucleic acids long. The single stranded region on the O oligonucleotide can be about 69 nucleic acids long. The single stranded region on the O oligonucleotide can be about 70 nucleic acids long. The single stranded region on the O oligonucleotide can be about 71 nucleic acids long. The single stranded region on the O oligonucleotide can be about 72 nucleic acids long. The single stranded region on the O oligonucleotide can be about 73 nucleic acids long. The single stranded region on the O oligonucleotide can be about 74 nucleic acids long. The single stranded region on the O oligonucleotide can be about 75 nucleic acids long. The single stranded region on the O oligonucleotide can be about 76 nucleic acids long. The single stranded region on the O oligonucleotide can be about 77 nucleic acids long. The single stranded region on the O oligonucleotide can be about 78 nucleic acids long. The single stranded region on the O oligonucleotide can be about 79 nucleic acids long. The single stranded region on the O oligonucleotide can be about 80 nucleic acids long. The single stranded region on the O oligonucleotide can be about 81 nucleic acids long. The single stranded region on the O oligonucleotide can be about 82 nucleic acids long. The single stranded region on the O oligonucleotide can be about 83 nucleic acids long. The single stranded region on the O oligonucleotide can be about 84 nucleic acids long. The single stranded region on the O oligonucleotide can be about 85 nucleic acids long. The single stranded region on the O oligonucleotide can be about 86 nucleic acids long. The single stranded region on the O oligonucleotide can be about 87 nucleic acids long. The single stranded region on the O oligonucleotide can be about 88 nucleic acids long. The single stranded region on the O oligonucleotide can be about 89 nucleic acids long. The single stranded region on the O oligonucleotide can be about 90 nucleic acids long. The single stranded region on the O oligonucleotide can be about 91 nucleic acids long. The single stranded region on the O oligonucleotide can be about 92 nucleic acids long. The single stranded region on the O oligonucleotide can be about 93 nucleic acids long. The single stranded region on the O oligonucleotide can be about 94 nucleic acids long. The single stranded region on the O oligonucleotide can be about 95 nucleic acids long. The single stranded region on the O oligonucleotide can be about 96 nucleic acids long. The single stranded region on the O oligonucleotide can be about 97 nucleic acids long. The single stranded region on the O oligonucleotide can be about 98 nucleic acids long. The single stranded region on the O oligonucleotide can be about 99 nucleic acids long. The single stranded region on the O oligonucleotide can be about 100 nucleic acids long. The single stranded region on the O oligonucleotide can be about 101 nucleic acids long. The single stranded region on the O oligonucleotide can be about 102 nucleic acids long. The single stranded region on the O oligonucleotide can be about 103 nucleic acids long. The single stranded region on the O oligonucleotide can be about 104 nucleic acids long. The single stranded region on the O oligonucleotide can be about 105 nucleic acids long. The single stranded region on the O oligonucleotide can be about 106 nucleic acids long. The single stranded region on the O oligonucleotide can be about 107 nucleic acids long. The single stranded region on the O oligonucleotide can be about 108 nucleic acids long. The single stranded region on the O oligonucleotide can be about 109 nucleic acids long. The single stranded region on the O oligonucleotide can be about 110 nucleic acids long. The single stranded region on the O oligonucleotide can be about 111 nucleic acids long. The single stranded region on the O oligonucleotide can be about 112 nucleic acids long. The single stranded region on the O oligonucleotide can be about 113 nucleic acids long. The single stranded region on the O oligonucleotide can be about 114 nucleic acids long. The single stranded region on the O oligonucleotide can be about 115 nucleic acids long. The single stranded region on the O oligonucleotide can be about 116 nucleic acids long. The single stranded region on the O oligonucleotide can be about 117 nucleic acids long. The single stranded region on the O oligonucleotide can be about 118 nucleic acids long. The single stranded region on the O oligonucleotide can be about 119 nucleic acids long. The single stranded region on the O oligonucleotide can be about 120 nucleic acids long. The single stranded region on the O oligonucleotide can be about 121 nucleic acids long. The single stranded region on the O oligonucleotide can be about 122 nucleic acids long. The single stranded region on the O oligonucleotide can be about 123 nucleic acids long. The single stranded region on the O oligonucleotide can be about 124 nucleic acids long. The single stranded region on the O oligonucleotide can be about 125 nucleic acids long. The single stranded region on the O oligonucleotide can be about 126 nucleic acids long. The single stranded region on the O oligonucleotide can be about 127 nucleic acids long. The single stranded region on the O oligonucleotide can be about 128 nucleic acids long. The single stranded region on the O oligonucleotide can be about 129 nucleic acids long. The single stranded region on the O oligonucleotide can be about 130 nucleic acids long. The single stranded region on the O oligonucleotide can be about 131 nucleic acids long. The single stranded region on the O oligonucleotide can be about 132 nucleic acids long. The single stranded region on the O oligonucleotide can be about 133 nucleic acids long. The single stranded region on the O oligonucleotide can be about 134 nucleic acids long. The single stranded region on the O oligonucleotide can be about 135 nucleic acids long. The single stranded region on the O oligonucleotide can be about 136 nucleic acids long. The single stranded region on the O oligonucleotide can be about 137 nucleic acids long. The single stranded region on the O oligonucleotide can be about 138 nucleic acids long. The single stranded region on the O oligonucleotide can be about 139 nucleic acids long. The single stranded region on the O oligonucleotide can be about 140 nucleic acids long. The single stranded region on the O oligonucleotide can be about 141 nucleic acids long. The single stranded region on the O oligonucleotide can be about 142 nucleic acids long. The single stranded region on the O oligonucleotide can be about 143 nucleic acids long. The single stranded region on the O oligonucleotide can be about 144 nucleic acids long. The single stranded region on the O oligonucleotide can be about 145 nucleic acids long. The single stranded region on the O oligonucleotide can be about 146 nucleic acids long. The single stranded region on the O oligonucleotide can be about 147 nucleic acids long. The single stranded region on the O oligonucleotide can be about 148 nucleic acids long. The single stranded region on the O oligonucleotide can be about 149 nucleic acids long. The single stranded region on the O oligonucleotide can be about 150 nucleic acids long. The single stranded region on the O oligonucleotide can be about 151 nucleic acids long. The single stranded region on the O oligonucleotide can be about 152 nucleic acids long. The single stranded region on the O oligonucleotide can be about 153 nucleic acids long. The single stranded region on the O oligonucleotide can be about 154 nucleic acids long. The single stranded region on the O oligonucleotide can be about 155 nucleic acids long. The single stranded region on the O oligonucleotide can be about 156 nucleic acids long. The single stranded region on the O oligonucleotide can be about 157 nucleic acids long. The single stranded region on the O oligonucleotide can be about 158 nucleic acids long. The single stranded region on the O oligonucleotide can be about 159 nucleic acids long. The single stranded region on the O oligonucleotide can be about 160 nucleic acids long. The single stranded region on the O oligonucleotide can be about 161 nucleic acids long. The single stranded region on the O oligonucleotide can be about 162 nucleic acids long. The single stranded region on the O oligonucleotide can be about 163 nucleic acids long. The single stranded region on the O oligonucleotide can be about 164 nucleic acids long. The single stranded region on the O oligonucleotide can be about 165 nucleic acids long. The single stranded region on the O oligonucleotide can be about 166 nucleic acids long. The single stranded region on the O oligonucleotide can be about 167 nucleic acids long. The single stranded region on the O oligonucleotide can be about 168 nucleic acids long. The single stranded region on the O oligonucleotide can be about 169 nucleic acids long. The single stranded region on the O oligonucleotide can be about 170 nucleic acids long. The single stranded region on the O oligonucleotide can be about 171 nucleic acids long. The single stranded region on the O oligonucleotide can be about 172 nucleic acids long. The single stranded region on the O oligonucleotide can be about 173 nucleic acids long. The single stranded region on the O oligonucleotide can be about 174 nucleic acids long. The single stranded region on the O oligonucleotide can be about 175 nucleic acids long. The single stranded region on the O oligonucleotide can be about 176 nucleic acids long. The single stranded region on the O oligonucleotide can be about 177 nucleic acids long. The single stranded region on the O oligonucleotide can be about 178 nucleic acids long. The single stranded region on the O oligonucleotide can be about 179 nucleic acids long. The single stranded region on the O oligonucleotide can be about 180 nucleic acids long. The single stranded region on the A oligonucleotide can be about 5 to about 180 nucleic acids long. The single stranded region on the A oligonucleotide can be about 10 to about 175 nucleic acids long. The single stranded region on the A oligonucleotide can be about 15 to about 170 nucleic acids long. The single stranded region on the A oligonucleotide can be about 20 to about 165 nucleic acids long. The single stranded region on the A oligonucleotide can be about 25 to about 160 nucleic acids long. The single stranded region on the A oligonucleotide can be about 30 to about 155 nucleic acids long. The single stranded region on the A oligonucleotide can be about 35 to about 150 nucleic acids long. The single stranded region on the A oligonucleotide can be about 40 to about 145 nucleic acids long. The single stranded region on the A oligonucleotide can be about 45 to about 140 nucleic acids long. The single stranded region on the A oligonucleotide can be about 50 to about 135 nucleic acids long. The single stranded region on the A oligonucleotide can be about 55 to about 130 nucleic acids long. The single stranded region on the A oligonucleotide can be about 60 to about 125 nucleic acids long. The single stranded region on the A oligonucleotide can be about 65 to about 120 nucleic acids long. The single stranded region on the A oligonucleotide can be about 70 to about 115 nucleic acids long. The single stranded region on the A oligonucleotide can be about 75 to about 110 nucleic acids long. The single stranded region on the A oligonucleotide can be about 80 to about 105 nucleic acids long. The single stranded region on the A oligonucleotide can be about 85 to about 100 nucleic acids long. The single stranded region on the A oligonucleotide can be about 90 to about 95 nucleic acids long.

### Toehold Region/Overhang

In some embodiments, a nucleic acid comprising part of an antigen binder (e.g. a partially double-stranded nucleic acid) may comprise an overhang (e.g. an overhanging single-stranded region configured to hybridize to another nucleic acid molecule). An overhang may be referred to as a toehold. In some embodiments, the overhang defines the way the sequences assemble. An overhang sequence may be designed to be orthogonal. In some embodiments, the orthogonal design of an overhang is relative to the design of other overhangs. In some embodiments, a partially double-stranded nucleic acid may comprise an overhang. In some embodiments, an overhang may allow two partially double-stranded nucleic acids to come together. In some embodiments, the two partially double-stranded nucleic acids have overhangs of different lengths. In some embodiments, the two partially double-stranded nucleic acids have overhangs of the same lengths. In some embodiments, the overhang is on the 5′ end of a nucleic acid. In some embodiments, the overhang is on the 3′ end of a nucleic acid. An overhang may be 4 nucleic acids in length to 15 nucleic acids in length. An overhang may be 4 nucleic acids in length to 5 nucleic acids in length, 4 nucleic acids in length to 6 nucleic acids in length, 4 nucleic acids in length to 7 nucleic acids in length, 4 nucleic acids in length to 8 nucleic acids in length, 4 nucleic acids in length to 9 nucleic acids in length, 4 nucleic acids in length to 10 nucleic acids in length, 4 nucleic acids in length to 11 nucleic acids in length, 4 nucleic acids in length to 12 nucleic acids in length, 4 nucleic acids in length to 13 nucleic acids in length, 4 nucleic acids in length to 14 nucleic acids in length, 4 nucleic acids in length to 15 nucleic acids in length, 5 nucleic acids in length to 6 nucleic acids in length, 5 nucleic acids in length to 7 nucleic acids in length, 5 nucleic acids in length to 8 nucleic acids in length, 5 nucleic acids in length to 9 nucleic acids in length, 5 nucleic acids in length to 10 nucleic acids in length, 5 nucleic acids in length to 11 nucleic acids in length, 5 nucleic acids in length to 12 nucleic acids in length, 5 nucleic acids in length to 13 nucleic acids in length, 5 nucleic acids in length to 14 nucleic acids in length, 5 nucleic acids in length to 15 nucleic acids in length, 6 nucleic acids in length to 7 nucleic acids in length, 6 nucleic acids in length to 8 nucleic acids in length, 6 nucleic acids in length to 9 nucleic acids in length, 6 nucleic acids in length to 10 nucleic acids in length, 6 nucleic acids in length to 11 nucleic acids in length, 6 nucleic acids in length to 12 nucleic acids in length, 6 nucleic acids in length to 13 nucleic acids in length, 6 nucleic acids in length to 14 nucleic acids in length, 6 nucleic acids in length to 15 nucleic acids in length, 7 nucleic acids in length to 8 nucleic acids in length, 7 nucleic acids in length to 9 nucleic acids in length, 7 nucleic acids in length to 10 nucleic acids in length, 7 nucleic acids in length to 11 nucleic acids in length, 7 nucleic acids in length to 12 nucleic acids in length, 7 nucleic acids in length to 13 nucleic acids in length, 7 nucleic acids in length to 14 nucleic acids in length, 7 nucleic acids in length to 15 nucleic acids in length, 8 nucleic acids in length to 9 nucleic acids in length, 8 nucleic acids in length to 10 nucleic acids in length, 8 nucleic acids in length to 11 nucleic acids in length, 8 nucleic acids in length to 12 nucleic acids in length, 8 nucleic acids in length to 13 nucleic acids in length, 8 nucleic acids in length to 14 nucleic acids in length, 8 nucleic acids in length to 15 nucleic acids in length, 9 nucleic acids in length to 10 nucleic acids in length, 9 nucleic acids in length to 11 nucleic acids in length, 9 nucleic acids in length to 12 nucleic acids in length, 9 nucleic acids in length to 13 nucleic acids in length, 9 nucleic acids in length to 14 nucleic acids in length, 9 nucleic acids in length to 15 nucleic acids in length, 10 nucleic acids in length to 11 nucleic acids in length, 10 nucleic acids in length to 12 nucleic acids in length, 10 nucleic acids in length to 13 nucleic acids in length, 10 nucleic acids in length to 14 nucleic acids in length, 10 nucleic acids in length to 15 nucleic acids in length, or 11 nucleic acids in length to 12 nucleic acids in length, 11 nucleic acids in length to 13 nucleic acids in length, 11 nucleic acids in length to 14 nucleic acids in length, 11 nucleic acids in length to 15 nucleic acids in length, 12 nucleic acids in length to 13 nucleic acids in length, 12 nucleic acids in length to 14 nucleic acids in length, 12 nucleic acids in length to 15 nucleic acids in length, 13 nucleic acids in length to 14 nucleic acids in length, 13 nucleic acids in length to 15 nucleic acids in length, or 14 nucleic acids in length to 15 nucleic acids in length. An overhang may be 5 nucleic acids in length to 14 nucleic acids in length. An overhang may be 6 nucleic acids in length to 13 nucleic acids in length. An overhang may be 8 nucleic acids in length to 12 nucleic acids in length. An overhang may be 9 nucleic acids in length to 11 nucleic acids in length. An overhang may be 7 nucleic acids in length to 9 nucleic acids in length. An overhang may be 4 nucleic acids in length, 5 nucleic acids in length, 6 nucleic acids in length, 7 nucleic acids in length, 8 nucleic acids in length, 9 nucleic acids in length, 10 nucleic acids in length, 11 nucleic acids in length, 12 nucleic acids in length, 13 nucleic acids in length, 14 nucleic acids in length, or 15 nucleic acids in length. An overhang may be at least 4 nucleic acids in length, 5 nucleic acids in length, 6 nucleic acids in length, 7 nucleic acids in length, 8 nucleic acids in length, 9 nucleic acids in length, 10 nucleic acids in length, 11 nucleic acids in length, 12 nucleic acids in length, 13 nucleic acids in length, or 14 nucleic acids in length. An overhang may be at most 5 nucleic acids in length, 6 nucleic acids in length, 7 nucleic acids in length, 8 nucleic acids in length, 9 nucleic acids in length, 10 nucleic acids in length, 11 nucleic acids in length, 12 nucleic acids in length, 13 nucleic acids in length, 14 nucleic acids in length, or 15 nucleic acids in length. An overhang may be about 14 nucleic acids in length to about 34 nucleic acids in length. An overhang may be about 14 nucleic acids in length to about 16 nucleic acids in length, about 14 nucleic acids in length to about 18 nucleic acids in length, about 14 nucleic acids in length to about 20 nucleic acids in length, about 14 nucleic acids in length to about 22 nucleic acids in length, about 14 nucleic acids in length to about 24 nucleic acids in length, about 14 nucleic acids in length to about 25 nucleic acids in length, about 14 nucleic acids in length to about 26 nucleic acids in length, about 14 nucleic acids in length to about 28 nucleic acids in length, about 14 nucleic acids in length to about 30 nucleic acids in length, about 14 nucleic acids in length to about 32 nucleic acids in length, about 14 nucleic acids in length to about 34 nucleic acids in length, about 16 nucleic acids in length to about 18 nucleic acids in length, about 16 nucleic acids in length to about 20 nucleic acids in length, about 16 nucleic acids in length to about 22 nucleic acids in length, about 16 nucleic acids in length to about 24 nucleic acids in length, about 16 nucleic acids in length to about 25 nucleic acids in length, about 16 nucleic acids in length to about 26 nucleic acids in length, about 16 nucleic acids in length to about 28 nucleic acids in length, about 16 nucleic acids in length to about 30 nucleic acids in length, about 16 nucleic acids in length to about 32 nucleic acids in length, about 16 nucleic acids in length to about 34 nucleic acids in length, about 18 nucleic acids in length to about 20 nucleic acids in length, about 18 nucleic acids in length to about 22 nucleic acids in length, about 18 nucleic acids in length to about 24 nucleic acids in length, about 18 nucleic acids in length to about 25 nucleic acids in length, about 18 nucleic acids in length to about 26 nucleic acids in length, about 18 nucleic acids in length to about 28 nucleic acids in length, about 18 nucleic acids in length to about 30 nucleic acids in length, about 18 nucleic acids in length to about 32 nucleic acids in length, about 18 nucleic acids in length to about 34 nucleic acids in length, about 20 nucleic acids in length to about 22 nucleic acids in length, about 20 nucleic acids in length to about 24 nucleic acids in length, about 20 nucleic acids in length to about 25 nucleic acids in length, about 20 nucleic acids in length to about 26 nucleic acids in length, about 20 nucleic acids in length to about 28 nucleic acids in length, about 20 nucleic acids in length to about 30 nucleic acids in length, about 20 nucleic acids in length to about 32 nucleic acids in length, about 20 nucleic acids in length to about 34 nucleic acids in length, about 22 nucleic acids in length to about 24 nucleic acids in length, about 22 nucleic acids in length to about 25 nucleic acids in length, about 22 nucleic acids in length to about 26 nucleic acids in length, about 22 nucleic acids in length to about 28 nucleic acids in length, about 22 nucleic acids in length to about 30 nucleic acids in length, about 22 nucleic acids in length to about 32 nucleic acids in length, about 22 nucleic acids in length to about 34 nucleic acids in length, about 24 nucleic acids in length to about 25 nucleic acids in length, about 24 nucleic acids in length to about 26 nucleic acids in length, about 24 nucleic acids in length to about 28 nucleic acids in length, about 24 nucleic acids in length to about 30 nucleic acids in length, about 24 nucleic acids in length to about 32 nucleic acids in length, about 24 nucleic acids in length to about 34 nucleic acids in length, about 25 nucleic acids in length to about 26 nucleic acids in length, about 25 nucleic acids in length to about 28 nucleic acids in length, about 25 nucleic acids in length to about 30 nucleic acids in length, about 25 nucleic acids in length to about 32 nucleic acids in length, about 25 nucleic acids in length to about 34 nucleic acids in length, about 26 nucleic acids in length to about 28 nucleic acids in length, about 26 nucleic acids in length to about 30 nucleic acids in length, about 26 nucleic acids in length to about 32 nucleic acids in length, about 26 nucleic acids in length to about 34 nucleic acids in length, about 28 nucleic acids in length to about 30 nucleic acids in length, about 28 nucleic acids in length to about 32 nucleic acids in length, about 28 nucleic acids in length to about 34 nucleic acids in length, about 30 nucleic acids in length to about 32 nucleic acids in length, about 30 nucleic acids in length to about 34 nucleic acids in length, or about 32 nucleic acids in length to about 34 nucleic acids in length. An overhang may be about 14 nucleic acids in length, about 16 nucleic acids in length, about 18 nucleic acids in length, about 20 nucleic acids in length, about 22 nucleic acids in length, about 24 nucleic acids in length, about 25 nucleic acids in length, about 26 nucleic acids in length, about 28 nucleic acids in length, about 30 nucleic acids in length, about 32 nucleic acids in length, or about 34 nucleic acids in length. An overhang may be at least about 14 nucleic acids in length, about 16 nucleic acids in length, about 18 nucleic acids in length, about 20 nucleic acids in length, about 22 nucleic acids in length, about 24 nucleic acids in length, about 25 nucleic acids in length, about 26 nucleic acids in length, about 28 nucleic acids in length, about 30 nucleic acids in length, or about 32 nucleic acids in length. An overhang may be at most about 16 nucleic acids in length, about 18 nucleic acids in length, about 20 nucleic acids in length, about 22 nucleic acids in length, about 24 nucleic acids in length, about 25 nucleic acids in length, about 26 nucleic acids in length, about 28 nucleic acids in length, about 30 nucleic acids in length, about 32 nucleic acids in length, or about 34 nucleic acids in length.

In some embodiments, an overhang of a nucleic acid included as part of an antigen binder can be configured to facilitate formation of a detectable nucleic acid. In some embodiments, an overhang hybridizes to another sequence. In some embodiments, an overhang hybridizes to another overhang. In some embodiments, the hybridization of overhangs results in a linear product. In some embodiments, the hybridization of overhangs results in a circular product.

### Common Hybridization Region

In some embodiments, a nucleic acid comprising part of an antigen binder (e.g. a partially double-stranded nucleic acid) can comprise a common hybridization region. In some embodiments, a common hybridization region is a part of an oligonucleotide. The common hybridization region may comprise 8 to 132 nucleic acids. A hybridization region may comprise about 8 nucleic acids to about 52 nucleic acids. A hybridization region may comprise about 8 nucleic acids to about 12 nucleic acids, about 8 nucleic acids to about 16 nucleic acids, about 8 nucleic acids to about 20 nucleic acids, about 8 nucleic acids to about 24 nucleic acids, about 8 nucleic acids to about 28 nucleic acids, about 8 nucleic acids to about 32 nucleic acids, about 8 nucleic acids to about 36 nucleic acids, about 8 nucleic acids to about 40 nucleic acids, about 8 nucleic acids to about 44 nucleic acids, about 8 nucleic acids to about 48 nucleic acids, about 8 nucleic acids to about 52 nucleic acids, about 12 nucleic acids to about 16 nucleic acids, about 12 nucleic acids to about 20 nucleic acids, about 12 nucleic acids to about 24 nucleic acids, about 12 nucleic acids to about 28 nucleic acids, about 12 nucleic acids to about 32 nucleic acids, about 12 nucleic acids to about 36 nucleic acids, about 12 nucleic acids to about 40 nucleic acids, about 12 nucleic acids to about 44 nucleic acids, about 12 nucleic acids to about 48 nucleic acids, about 12 nucleic acids to about 52 nucleic acids, about 16 nucleic acids to about 20 nucleic acids, about 16 nucleic acids to about 24 nucleic acids, about 16 nucleic acids to about 28 nucleic acids, about 16 nucleic acids to about 32 nucleic acids, about 16 nucleic acids to about 36 nucleic acids, about 16 nucleic acids to about 40 nucleic acids, about 16 nucleic acids to about 44 nucleic acids, about 16 nucleic acids to about 48 nucleic acids, about 16 nucleic acids to about 52 nucleic acids, about 20 nucleic acids to about 24 nucleic acids, about 20 nucleic acids to about 28 nucleic acids, about 20 nucleic acids to about 32 nucleic acids, about 20 nucleic acids to about 36 nucleic acids, about 20 nucleic acids to about 40 nucleic acids, about 20 nucleic acids to about 44 nucleic acids, about 20 nucleic acids to about 48 nucleic acids, about 20 nucleic acids to about 52 nucleic acids, about 24 nucleic acids to about 28 nucleic acids, about 24 nucleic acids to about 32 nucleic acids, about 24 nucleic acids to about 36 nucleic acids, about 24 nucleic acids to about 40 nucleic acids, about 24 nucleic acids to about 44 nucleic acids, about 24 nucleic acids to about 48 nucleic acids, about 24 nucleic acids to about 52 nucleic acids, about 28 nucleic acids to about 32 nucleic acids, about 28 nucleic acids to about 36 nucleic acids, about 28 nucleic acids to about 40 nucleic acids, about 28 nucleic acids to about 44 nucleic acids, about 28 nucleic acids to about 48 nucleic acids, about 28 nucleic acids to about 52 nucleic acids, about 32 nucleic acids to about 36 nucleic acids, about 32 nucleic acids to about 40 nucleic acids, about 32 nucleic acids to about 44 nucleic acids, about 32 nucleic acids to about 48 nucleic acids, about 32 nucleic acids to about 52 nucleic acids, about 36 nucleic acids to about 40 nucleic acids, about 36 nucleic acids to about 44 nucleic acids, about 36 nucleic acids to about 48 nucleic acids, about 36 nucleic acids to about 52 nucleic acids, about 40 nucleic acids to about 44 nucleic acids, about 40 nucleic acids to about 48 nucleic acids, about 40 nucleic acids to about 52 nucleic acids, about 44 nucleic acids to about 48 nucleic acids, about 44 nucleic acids to about 52 nucleic acids, or about 48 nucleic acids to about 52 nucleic acids. A hybridization region may comprise about 8 nucleic acids, about 12 nucleic acids, about 16 nucleic acids, about 20 nucleic acids, about 24 nucleic acids, about 28 nucleic acids, about 32 nucleic acids, about 36 nucleic acids, about 40 nucleic acids, about 44 nucleic acids, about 48 nucleic acids, or about 52 nucleic acids. A hybridization region may comprise at least about 8 nucleic acids, about 12 nucleic acids, about 16 nucleic acids, about 20 nucleic acids, about 24 nucleic acids, about 28 nucleic acids, about 32 nucleic acids, about 36 nucleic acids, about 40 nucleic acids, about 44 nucleic acids, or about 48 nucleic acids. A hybridization region may comprise at most about 12 nucleic acids, about 16 nucleic acids, about 20 nucleic acids, about 24 nucleic acids, about 28 nucleic acids, about 32 nucleic acids, about 36 nucleic acids, about 40 nucleic acids, about 44 nucleic acids, about 48 nucleic acids, or about 52 nucleic acids. A hybridization region may comprise about 8 nucleic acids to about 100 nucleic acids. A hybridization region may comprise about 56 nucleic acids to about 60 nucleic acids, about 56 nucleic acids to about 64 nucleic acids, about 56 nucleic acids to about 68 nucleic acids, about 56 nucleic acids to about 72 nucleic acids, about 56 nucleic acids to about 76 nucleic acids, about 56 nucleic acids to about 80 nucleic acids, about 56 nucleic acids to about 84 nucleic acids, about 56 nucleic acids to about 88 nucleic acids, about 56 nucleic acids to about 92 nucleic acids, about 56 nucleic acids to about 96 nucleic acids, about 56 nucleic acids to about 100 nucleic acids, about 60 nucleic acids to about 64 nucleic acids, about 60 nucleic acids to about 68 nucleic acids, about 60 nucleic acids to about 72 nucleic acids, about 60 nucleic acids to about 76 nucleic acids, about 60 nucleic acids to about 80 nucleic acids, about 60 nucleic acids to about 84 nucleic acids, about 60 nucleic acids to about 88 nucleic acids, about 60 nucleic acids to about 92 nucleic acids, about 60 nucleic acids to about 96 nucleic acids, about 60 nucleic acids to about 100 nucleic acids, about 64 nucleic acids to about 68 nucleic acids, about 64 nucleic acids to about 72 nucleic acids, about 64 nucleic acids to about 76 nucleic acids, about 64 nucleic acids to about 80 nucleic acids, about 64 nucleic acids to about 84 nucleic acids, about 64 nucleic acids to about 88 nucleic acids, about 64 nucleic acids to about 92 nucleic acids, about 64 nucleic acids to about 96 nucleic acids, about 64 nucleic acids to about 100 nucleic acids, about 68 nucleic acids to about 72 nucleic acids, about 68 nucleic acids to about 76 nucleic acids, about 68 nucleic acids to about 80 nucleic acids, about 68 nucleic acids to about 84 nucleic acids, about 68 nucleic acids to about 88 nucleic acids, about 68 nucleic acids to about 92 nucleic acids, about 68 nucleic acids to about 96 nucleic acids, about 68 nucleic acids to about 100 nucleic acids, about 72 nucleic acids to about 76 nucleic acids, about 72 nucleic acids to about 80 nucleic acids, about 72 nucleic acids to about 84 nucleic acids, about 72 nucleic acids to about 88 nucleic acids, about 72 nucleic acids to about 92 nucleic acids, about 72 nucleic acids to about 96 nucleic acids, about 72 nucleic acids to about 100 nucleic acids, about 76 nucleic acids to about 80 nucleic acids, about 76 nucleic acids to about 84 nucleic acids, about 76 nucleic acids to about 88 nucleic acids, about 76 nucleic acids to about 92 nucleic acids, about 76 nucleic acids to about 96 nucleic acids, about 76 nucleic acids to about 100 nucleic acids, about 80 nucleic acids to about 84 nucleic acids, about 80 nucleic acids to about 88 nucleic acids, about 80 nucleic acids to about 92 nucleic acids, about 80 nucleic acids to about 96 nucleic acids, about 80 nucleic acids to about 100 nucleic acids, about 84 nucleic acids to about 88 nucleic acids, about 84 nucleic acids to about 92 nucleic acids, about 84 nucleic acids to about 96 nucleic acids, about 84 nucleic acids to about 100 nucleic acids, about 88 nucleic acids to about 92 nucleic acids, about 88 nucleic acids to about 96 nucleic acids, about 88 nucleic acids to about 100 nucleic acids, about 92 nucleic acids to about 96 nucleic acids, about 92 nucleic acids to about 100 nucleic acids, or about 96 nucleic acids to about 100 nucleic acids. A hybridization region may comprise about 56 nucleic acids, about 60 nucleic acids, about 64 nucleic acids, about 68 nucleic acids, about 72 nucleic acids, about 76 nucleic acids, about 80 nucleic acids, about 84 nucleic acids, about 88 nucleic acids, about 92 nucleic acids, about 96 nucleic acids, or about 100 nucleic acids. A hybridization region may comprise at least about 56 nucleic acids, about 60 nucleic acids, about 64 nucleic acids, about 68 nucleic acids, about 72 nucleic acids, about 76 nucleic acids, about 80 nucleic acids, about 84 nucleic acids, about 88 nucleic acids, about 92 nucleic acids, or about 96 nucleic acids. A hybridization region may comprise at most about 60 nucleic acids, about 64 nucleic acids, about 68 nucleic acids, about 72 nucleic acids, about 76 nucleic acids, about 80 nucleic acids, about 84 nucleic acids, about 88 nucleic acids, about 92 nucleic acids, about 96 nucleic acids, or about 100 nucleic acids. A hybridization region may comprise about 8 nucleic acids to about 132 nucleic acids. A hybridization region may comprise about 104 nucleic acids to about 108 nucleic acids, about 104 nucleic acids to about 112 nucleic acids, about 104 nucleic acids to about 116 nucleic acids, about 104 nucleic acids to about 120 nucleic acids, about 104 nucleic acids to about 124 nucleic acids, about 104 nucleic acids to about 128 nucleic acids, about 104 nucleic acids to about 132 nucleic acids, about 108 nucleic acids to about 112 nucleic acids, about 108 nucleic acids to about 116 nucleic acids, about 108 nucleic acids to about 120 nucleic acids, about 108 nucleic acids to about 124 nucleic acids, about 108 nucleic acids to about 128 nucleic acids, about 108 nucleic acids to about 132 nucleic acids, about 112 nucleic acids to about 116 nucleic acids, about 112 nucleic acids to about 120 nucleic acids, about 112 nucleic acids to about 124 nucleic acids, about 112 nucleic acids to about 128 nucleic acids, about 112 nucleic acids to about 132 nucleic acids, about 116 nucleic acids to about 120 nucleic acids, about 116 nucleic acids to about 124 nucleic acids, about 116 nucleic acids to about 128 nucleic acids, about 116 nucleic acids to about 132 nucleic acids, about 120 nucleic acids to about 124 nucleic acids, about 120 nucleic acids to about 128 nucleic acids, about 120 nucleic acids to about 132 nucleic acids, about 124 nucleic acids to about 128 nucleic acids, about 124 nucleic acids to about 132 nucleic acids, or about 128 nucleic acids to about 132 nucleic acids. A hybridization region may comprise about 104 nucleic acids, about 108 nucleic acids, about 112 nucleic acids, about 116 nucleic acids, about 120 nucleic acids, about 124 nucleic acids, about 128 nucleic acids, or about 132 nucleic acids. A hybridization region may comprise at least about 104 nucleic acids, about 108 nucleic acids, about 112 nucleic acids, about 116 nucleic acids, about 120 nucleic acids, about 124 nucleic acids, or about 128 nucleic acids. A hybridization region may comprise at most about 108 nucleic acids, about 112 nucleic acids, about 116 nucleic acids, about 120 nucleic acids, about 124 nucleic acids, about 128 nucleic acids, or about 132 nucleic acids. A hybridization region may comprise about 20 nucleic acids to about 30 nucleic acids. A hybridization region may comprise about 20 nucleic acids to about 21 nucleic acids, about 20 nucleic acids to about 22 nucleic acids, about 20 nucleic acids to about 23 nucleic acids, about 20 nucleic acids to about 24 nucleic acids, about 20 nucleic acids to about 25 nucleic acids, about 20 nucleic acids to about 26 nucleic acids, about 20 nucleic acids to about 27 nucleic acids, about 20 nucleic acids to about 28 nucleic acids, about 20 nucleic acids to about 29 nucleic acids, about 20 nucleic acids to about 30 nucleic acids, about 21 nucleic acids to about 22 nucleic acids, about 21 nucleic acids to about 23 nucleic acids, about 21 nucleic acids to about 24 nucleic acids, about 21 nucleic acids to about 25 nucleic acids, about 21 nucleic acids to about 26 nucleic acids, about 21 nucleic acids to about 27 nucleic acids, about 21 nucleic acids to about 28 nucleic acids, about 21 nucleic acids to about 29 nucleic acids, about 21 nucleic acids to about 30 nucleic acids, about 22 nucleic acids to about 23 nucleic acids, about 22 nucleic acids to about 24 nucleic acids, about 22 nucleic acids to about 25 nucleic acids, about 22 nucleic acids to about 26 nucleic acids, about 22 nucleic acids to about 27 nucleic acids, about 22 nucleic acids to about 28 nucleic acids, about 22 nucleic acids to about 29 nucleic acids, about 22 nucleic acids to about 30 nucleic acids, about 23 nucleic acids to about 24 nucleic acids, about 23 nucleic acids to about 25 nucleic acids, about 23 nucleic acids to about 26 nucleic acids, about 23 nucleic acids to about 27 nucleic acids, about 23 nucleic acids to about 28 nucleic acids, about 23 nucleic acids to about 29 nucleic acids, about 23 nucleic acids to about 30 nucleic acids, about 24 nucleic acids to about 25 nucleic acids, about 24 nucleic acids to about 26 nucleic acids, about 24 nucleic acids to about 27 nucleic acids, about 24 nucleic acids to about 28 nucleic acids, about 24 nucleic acids to about 29 nucleic acids, about 24 nucleic acids to about 30 nucleic acids, about 25 nucleic acids to about 26 nucleic acids, about 25 nucleic acids to about 27 nucleic acids, about 25 nucleic acids to about 28 nucleic acids, about 25 nucleic acids to about 29 nucleic acids, about 25 nucleic acids to about 30 nucleic acids, about 26 nucleic acids to about 27 nucleic acids, about 26 nucleic acids to about 28 nucleic acids, about 26 nucleic acids to about 29 nucleic acids, about 26 nucleic acids to about 30 nucleic acids, about 27 nucleic acids to about 28 nucleic acids, about 27 nucleic acids to about 29 nucleic acids, about 27 nucleic acids to about 30 nucleic acids, about 28 nucleic acids to about 29 nucleic acids, about 28 nucleic acids to about 30 nucleic acids, or about 29 nucleic acids to about 30 nucleic acids. A hybridization region may comprise about 20 nucleic acids, about 21 nucleic acids, about 22 nucleic acids, about 23 nucleic acids, about 24 nucleic acids, about 25 nucleic acids, about 26 nucleic acids, about 27 nucleic acids, about 28 nucleic acids, about 29 nucleic acids, or about 30 nucleic acids. A hybridization region may comprise at least about 20 nucleic acids, about 21 nucleic acids, about 22 nucleic acids, about 23 nucleic acids, about 24 nucleic acids, about 25 nucleic acids, about 26 nucleic acids, about 27 nucleic acids, about 28 nucleic acids, or about 29 nucleic acids. A hybridization region may comprise at most about 21 nucleic acids, about 22 nucleic acids, about 23 nucleic acids, about 24 nucleic acids, about 25 nucleic acids, about 26 nucleic acids, about 27 nucleic acids, about 28 nucleic acids, about 29 nucleic acids, or about 30 nucleic acids.

A common hybridization region may be configured to hybridize at a particular temperature (e.g. have an optimized sequence designed to hybridize to another nucleic acid at a predetermined melting temperature or Tm). In some embodiments, the hybridization region has a melting temperature of 25° C. In some embodiments, the hybridization region has a melting temperature greater than 25° C.

In some embodiments, the oligonucleotide with a common hybridization region may comprise an optional region (e.g. a region to assist detection or processing of the nucleic acid which does not participate in direct function of the antigen binder(s) in detecting an antigen). In some embodiments, the oligonucleotide with the hybridization region may not have an optional region. The optional region may be 0 nucleotides. The optional region may be 100 nucleotides. The optional region may be greater than about 0 nucleotides. The optional region may be less than about 100 nucleotides. The optional region may be about 0 nucleotides to about 100 nucleotides. The optional region may be about 0 nucleotides to about 100 nucleotides. The optional region may be about 0 nucleotides to about 10 nucleotides, about 0 nucleotides to about 20 nucleotides, about 0 nucleotides to about 30 nucleotides, about 0 nucleotides to about 40 nucleotides, about 0 nucleotides to about 50 nucleotides, about 0 nucleotides to about 60 nucleotides, about 0 nucleotides to about 70 nucleotides, about 0 nucleotides to about 80 nucleotides, about 0 nucleotides to about 90 nucleotides, about 0 nucleotides to about 100 nucleotides, about 10 nucleotides to about 20 nucleotides, about 10 nucleotides to about 30 nucleotides, about 10 nucleotides to about 40 nucleotides, about 10 nucleotides to about 50 nucleotides, about 10 nucleotides to about 60 nucleotides, about 10 nucleotides to about 70 nucleotides, about 10 nucleotides to about 80 nucleotides, about 10 nucleotides to about 90 nucleotides, about 10 nucleotides to about 100 nucleotides, about 20 nucleotides to about 30 nucleotides, about 20 nucleotides to about 40 nucleotides, about 20 nucleotides to about 50 nucleotides, about 20 nucleotides to about 60 nucleotides, about 20 nucleotides to about 70 nucleotides, about 20 nucleotides to about 80 nucleotides, about 20 nucleotides to about 90 nucleotides, about 20 nucleotides to about 100 nucleotides, about 30 nucleotides to about 40 nucleotides, about 30 nucleotides to about 50 nucleotides, about 30 nucleotides to about 60 nucleotides, about 30 nucleotides to about 70 nucleotides, about 30 nucleotides to about 80 nucleotides, about 30 nucleotides to about 90 nucleotides, about 30 nucleotides to about 100 nucleotides, about 40 nucleotides to about 50 nucleotides, about 40 nucleotides to about 60 nucleotides, about 40 nucleotides to about 70 nucleotides, about 40 nucleotides to about 80 nucleotides, about 40 nucleotides to about 90 nucleotides, about 40 nucleotides to about 100 nucleotides, about 50 nucleotides to about 60 nucleotides, about 50 nucleotides to about 70 nucleotides, about 50 nucleotides to about 80 nucleotides, about 50 nucleotides to about 90 nucleotides, about 50 nucleotides to about 100 nucleotides, about 60 nucleotides to about 70 nucleotides, about 60 nucleotides to about 80 nucleotides, about 60 nucleotides to about 90 nucleotides, about 60 nucleotides to about 100 nucleotides, about 70 nucleotides to about 80 nucleotides, about 70 nucleotides to about 90 nucleotides, about 70 nucleotides to about 100 nucleotides, about 80 nucleotides to about 90 nucleotides, about 80 nucleotides to about 100 nucleotides, or about 90 nucleotides to about 100 nucleotides. The optional region may be about 0 nucleotides, about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, or about 100 nucleotides. The optional region may be at least about 0 nucleotides, about 10 nucleotides, about 20 nucleotides, about 30 nucleotides, about 40 nucleotides, about 50 nucleotides, about 60 nucleotides, about 70 nucleotides, about 80 nucleotides, or about 90 nucleotides. The optional region may be at most about 10 nucleotides, about 20 nucleotides, about 30 nucleotides, about 40 nucleotides, about 50 nucleotides, about 60 nucleotides, about 70 nucleotides, about 80 nucleotides, about 90 nucleotides, or about 100 nucleotides. The optional region may be about 0 nucleotides to about 100 nucleotides. The optional region may be about 0 nucleotides to about 5 nucleotides, about 0 nucleotides to about 15 nucleotides, about 0 nucleotides to about 25 nucleotides, about 0 nucleotides to about 35 nucleotides, about 0 nucleotides to about 45 nucleotides, about 0 nucleotides to about 55 nucleotides, about 0 nucleotides to about 65 nucleotides, about 0 nucleotides to about 75 nucleotides, about 0 nucleotides to about 85 nucleotides, about 0 nucleotides to about 95 nucleotides, about 0 nucleotides to about 100 nucleotides, about 5 nucleotides to about 15 nucleotides, about 5 nucleotides to about 25 nucleotides, about 5 nucleotides to about 35 nucleotides, about 5 nucleotides to about 45 nucleotides, about 5 nucleotides to about 55 nucleotides, about 5 nucleotides to about 65 nucleotides, about 5 nucleotides to about 75 nucleotides, about 5 nucleotides to about 85 nucleotides, about 5 nucleotides to about 95 nucleotides, about 5 nucleotides to about 100 nucleotides, about 15 nucleotides to about 25 nucleotides, about 15 nucleotides to about 35 nucleotides, about 15 nucleotides to about 45 nucleotides, about 15 nucleotides to about 55 nucleotides, about 15 nucleotides to about 65 nucleotides, about 15 nucleotides to about 75 nucleotides, about 15 nucleotides to about 85 nucleotides, about 15 nucleotides to about 95 nucleotides, about 15 nucleotides to about 100 nucleotides, about 25 nucleotides to about 35 nucleotides, about 25 nucleotides to about 45 nucleotides, about 25 nucleotides to about 55 nucleotides, about 25 nucleotides to about 65 nucleotides, about 25 nucleotides to about 75 nucleotides, about 25 nucleotides to about 85 nucleotides, about 25 nucleotides to about 95 nucleotides, about 25 nucleotides to about 100 nucleotides, about 35 nucleotides to about 45 nucleotides, about 35 nucleotides to about 55 nucleotides, about 35 nucleotides to about 65 nucleotides, about 35 nucleotides to about 75 nucleotides, about 35 nucleotides to about 85 nucleotides, about 35 nucleotides to about 95 nucleotides, about 35 nucleotides to about 100 nucleotides, about 45 nucleotides to about 55 nucleotides, about 45 nucleotides to about 65 nucleotides, about 45 nucleotides to about 75 nucleotides, about 45 nucleotides to about 85 nucleotides, about 45 nucleotides to about 95 nucleotides, about 45 nucleotides to about 100 nucleotides, about 55 nucleotides to about 65 nucleotides, about 55 nucleotides to about 75 nucleotides, about 55 nucleotides to about 85 nucleotides, about 55 nucleotides to about 95 nucleotides, about 55 nucleotides to about 100 nucleotides, about 65 nucleotides to about 75 nucleotides, about 65 nucleotides to about 85 nucleotides, about 65 nucleotides to about 95 nucleotides, about 65 nucleotides to about 100 nucleotides, about 75 nucleotides to about 85 nucleotides, about 75 nucleotides to about 95 nucleotides, about 75 nucleotides to about 100 nucleotides, about 85 nucleotides to about 95 nucleotides, about 85 nucleotides to about 100 nucleotides, or about 95 nucleotides to about 100 nucleotides. The optional region may be about 0 nucleotides, about 5 nucleotides, about 15 nucleotides, about 25 nucleotides, about 35 nucleotides, about 45 nucleotides, about 55 nucleotides, about 65 nucleotides, about 75 nucleotides, about 85 nucleotides, about 95 nucleotides, or about 100 nucleotides. The optional region may be at least about 0 nucleotides, about 5 nucleotides, about 15 nucleotides, about 25 nucleotides, about 35 nucleotides, about 45 nucleotides, about 55 nucleotides, about 65 nucleotides, about 75 nucleotides, about 85 nucleotides, or about 95 nucleotides. The optional region may be at most about 5 nucleotides, about 15 nucleotides, about 25 nucleotides, about 35 nucleotides, about 45 nucleotides, about 55 nucleotides, about 65 nucleotides, about 75 nucleotides, about 85 nucleotides, about 95 nucleotides, or about 100 nucleotides.

The optional region may comprise a primer hybridization region, sequencing adapter, an endonuclease site, a barcodes, or any combination thereof. The primer hybridization region may be a forward primers hybridization region, a reverse primers reverse primer hybridization region, or a bidirectional hybridization region. The primer hybridization region may be used for downstream sequencing operations, amplification operations, or detection (e.g. via probe hybridization in techniques such as qPCR). In some embodiments, the oligonucleotide with a hybridization region further comprises a unique molecular identifier (UMI), an index, a toehold, or any combination thereof. In some embodiments, the hybridization region is on one end of the oligonucleotide and the overhang is on the opposite end of the oligonucleotide. The index and the UMI may be next to one another in the oligonucleotide. The index and the UMI may be interwoven on the oligonucleotide. The index may be about 3 base pairs to about 24 base pairs. The index may be about 3 base pairs to about 4 base pairs, about 3 base pairs to about 6 base pairs, about 3 base pairs to about 7 base pairs, about 3 base pairs to about 8 base pairs, about 3 base pairs to about 9 base pairs, about 3 base pairs to about 10 base pairs, about 3 base pairs to about 12 base pairs, about 3 base pairs to about 14 base pairs, about 3 base pairs to about 16 base pairs, about 3 base pairs to about 18 base pairs, about 3 base pairs to about 20 base pairs, about 3 base pairs to about 22 base pairs, about 3 base pairs to about 24 base pairs, about 4 base pairs to about 6 base pairs, about 4 base pairs to about 7 base pairs, about 4 base pairs to about 8 base pairs, about 4 base pairs to about 9 base pairs, about 4 base pairs to about 10 base pairs, about 4 base pairs to about 12 base pairs, about 4 base pairs to about 14 base pairs, about 4 base pairs to about 16 base pairs, about 4 base pairs to about 18 base pairs, about 4 base pairs to about 20 base pairs, about 4 base pairs to about 22 base pairs, about 4 base pairs to about 24 base pairs, about 6 base pairs to about 7 base pairs, about 6 base pairs to about 8 base pairs, about 6 base pairs to about 9 base pairs, about 6 base pairs to about 10 base pairs, about 6 base pairs to about 12 base pairs, about 6 base pairs to about 14 base pairs, about 6 base pairs to about 16 base pairs, about 6 base pairs to about 18 base pairs, about 6 base pairs to about 20 base pairs, about 6 base pairs to about 22 base pairs, about 6 base pairs to about 24 base pairs, about 7 base pairs to about 8 base pairs, about 7 base pairs to about 9 base pairs, about 7 base pairs to about 10 base pairs, about 7 base pairs to about 12 base pairs, about 7 base pairs to about 14 base pairs, about 7 base pairs to about 16 base pairs, about 7 base pairs to about 18 base pairs, about 7 base pairs to about 20 base pairs, about 7 base pairs to about 22 base pairs, about 7 base pairs to about 24 base pairs, about 8 base pairs to about 9 base pairs, about 8 base pairs to about 10 base pairs, about 8 base pairs to about 12 base pairs, about 8 base pairs to about 14 base pairs, about 8 base pairs to about 16 base pairs, about 8 base pairs to about 18 base pairs, about 8 base pairs to about 20 base pairs, about 8 base pairs to about 22 base pairs, about 8 base pairs to about 24 base pairs, about 9 base pairs to about 10 base pairs, about 9 base pairs to about 12 base pairs, about 9 base pairs to about 14 base pairs, about 9 base pairs to about 16 base pairs, about 9 base pairs to about 18 base pairs, about 9 base pairs to about 20 base pairs, about 9 base pairs to about 22 base pairs, about 9 base pairs to about 24 base pairs, about 10 base pairs to about 12 base pairs, about 10 base pairs to about 14 base pairs, about 10 base pairs to about 16 base pairs, about 10 base pairs to about 18 base pairs, about 10 base pairs to about 20 base pairs, about 10 base pairs to about 22 base pairs, about 10 base pairs to about 24 base pairs, about 12 base pairs to about 14 base pairs, about 12 base pairs to about 16 base pairs, about 12 base pairs to about 18 base pairs, about 12 base pairs to about 20 base pairs, about 12 base pairs to about 22 base pairs, about 12 base pairs to about 24 base pairs, about 14 base pairs to about 16 base pairs, about 14 base pairs to about 18 base pairs, about 14 base pairs to about 20 base pairs, about 14 base pairs to about 22 base pairs, about 14 base pairs to about 24 base pairs, about 16 base pairs to about 18 base pairs, about 16 base pairs to about 20 base pairs, about 16 base pairs to about 22 base pairs, about 16 base pairs to about 24 base pairs, about 18 base pairs to about 20 base pairs, about 18 base pairs to about 22 base pairs, about 18 base pairs to about 24 base pairs, about 20 base pairs to about 22 base pairs, about 20 base pairs to about 24 base pairs, or about 22 base pairs to about 24 base pairs. The index may be about 3 base pairs, about 4 base pairs, about 6 base pairs, about 7 base pairs, about 8 base pairs, about 9 base pairs, about 10 base pairs, about 12 base pairs, about 14 base pairs, about 16 base pairs, about 18 base pairs, about 20 base pairs, about 22 base pairs, or about 24 base pairs. The index may be at least about 3 base pairs, about 4 base pairs, about 6 base pairs, about 7 base pairs, about 8 base pairs, about 9 base pairs, about 10 base pairs, about 12 base pairs, about 14 base pairs, about 16 base pairs, about 18 base pairs, about 20 base pairs, or about 22 base pairs. The index may be at most about 4 base pairs, about 6 base pairs, about 7 base pairs, about 8 base pairs, about 9 base pairs, about 10 base pairs, about 12 base pairs, about 14 base pairs, about 16 base pairs, about 18 base pairs, about 20 base pairs, about 22 base pairs, or about 24 base pairs. The UMI may be about 3 base pairs to about 24 base pairs. The UMI may be about 3 base pairs to about 4 base pairs, about 3 base pairs to about 6 base pairs, about 3 base pairs to about 7 base pairs, about 3 base pairs to about 8 base pairs, about 3 base pairs to about 9 base pairs, about 3 base pairs to about 10 base pairs, about 3 base pairs to about 12 base pairs, about 3 base pairs to about 14 base pairs, about 3 base pairs to about 16 base pairs, about 3 base pairs to about 18 base pairs, about 3 base pairs to about 20 base pairs, about 3 base pairs to about 22 base pairs, about 3 base pairs to about 24 base pairs, about 4 base pairs to about 6 base pairs, about 4 base pairs to about 7 base pairs, about 4 base pairs to about 8 base pairs, about 4 base pairs to about 9 base pairs, about 4 base pairs to about 10 base pairs, about 4 base pairs to about 12 base pairs, about 4 base pairs to about 14 base pairs, about 4 base pairs to about 16 base pairs, about 4 base pairs to about 18 base pairs, about 4 base pairs to about 20 base pairs, about 4 base pairs to about 22 base pairs, about 4 base pairs to about 24 base pairs, about 6 base pairs to about 7 base pairs, about 6 base pairs to about 8 base pairs, about 6 base pairs to about 9 base pairs, about 6 base pairs to about 10 base pairs, about 6 base pairs to about 12 base pairs, about 6 base pairs to about 14 base pairs, about 6 base pairs to about 16 base pairs, about 6 base pairs to about 18 base pairs, about 6 base pairs to about 20 base pairs, about 6 base pairs to about 22 base pairs, about 6 base pairs to about 24 base pairs, about 7 base pairs to about 8 base pairs, about 7 base pairs to about 9 base pairs, about 7 base pairs to about 10 base pairs, about 7 base pairs to about 12 base pairs, about 7 base pairs to about 14 base pairs, about 7 base pairs to about 16 base pairs, about 7 base pairs to about 18 base pairs, about 7 base pairs to about 20 base pairs, about 7 base pairs to about 22 base pairs, about 7 base pairs to about 24 base pairs, about 8 base pairs to about 9 base pairs, about 8 base pairs to about 10 base pairs, about 8 base pairs to about 12 base pairs, about 8 base pairs to about 14 base pairs, about 8 base pairs to about 16 base pairs, about 8 base pairs to about 18 base pairs, about 8 base pairs to about 20 base pairs, about 8 base pairs to about 22 base pairs, about 8 base pairs to about 24 base pairs, about 9 base pairs to about 10 base pairs, about 9 base pairs to about 12 base pairs, about 9 base pairs to about 14 base pairs, about 9 base pairs to about 16 base pairs, about 9 base pairs to about 18 base pairs, about 9 base pairs to about 20 base pairs, about 9 base pairs to about 22 base pairs, about 9 base pairs to about 24 base pairs, about 10 base pairs to about 12 base pairs, about 10 base pairs to about 14 base pairs, about 10 base pairs to about 16 base pairs, about 10 base pairs to about 18 base pairs, about 10 base pairs to about 20 base pairs, about 10 base pairs to about 22 base pairs, about 10 base pairs to about 24 base pairs, about 12 base pairs to about 14 base pairs, about 12 base pairs to about 16 base pairs, about 12 base pairs to about 18 base pairs, about 12 base pairs to about 20 base pairs, about 12 base pairs to about 22 base pairs, about 12 base pairs to about 24 base pairs, about 14 base pairs to about 16 base pairs, about 14 base pairs to about 18 base pairs, about 14 base pairs to about 20 base pairs, about 14 base pairs to about 22 base pairs, about 14 base pairs to about 24 base pairs, about 16 base pairs to about 18 base pairs, about 16 base pairs to about 20 base pairs, about 16 base pairs to about 22 base pairs, about 16 base pairs to about 24 base pairs, about 18 base pairs to about 20 base pairs, about 18 base pairs to about 22 base pairs, about 18 base pairs to about 24 base pairs, about 20 base pairs to about 22 base pairs, about 20 base pairs to about 24 base pairs, or about 22 base pairs to about 24 base pairs. The UMI may be about 3 base pairs, about 4 base pairs, about 6 base pairs, about 7 base pairs, about 8 base pairs, about 9 base pairs, about 10 base pairs, about 12 base pairs, about 14 base pairs, about 16 base pairs, about 18 base pairs, about 20 base pairs, about 22 base pairs, or about 24 base pairs. The UMI may be at least about 3 base pairs, about 4 base pairs, about 6 base pairs, about 7 base pairs, about 8 base pairs, about 9 base pairs, about 10 base pairs, about 12 base pairs, about 14 base pairs, about 16 base pairs, about 18 base pairs, about 20 base pairs, or about 22 base pairs. The UMI may be at most about 4 base pairs, about 6 base pairs, about 7 base pairs, about 8 base pairs, about 9 base pairs, about 10 base pairs, about 12 base pairs, about 14 base pairs, about 16 base pairs, about 18 base pairs, about 20 base pairs, about 22 base pairs, or about 24 base pairs. In some embodiments, both nucleic acids hybridizing to one another have unhybridized 5′ ends. In some embodiments, both nucleic acids hybridizing to one another have unhybridized 3′ ends.

An antigen binder may be attached to a solid surface or free-floating in solution. In some embodiments, an oligonucleotide is attached to a solid surface. In some embodiments, an antibody is attached to a solid surface. In some embodiments, an antigen is attached to a solid surface. In some embodiments, the solid surface is a spherical surface. In some embodiments, the solid surface is a bead. In some embodiments, the solid surface is a planar surface. In some embodiments, the solid surface is a resin. In some embodiments, the solid surface is a nanoparticle. In some embodiments, the solid surface is magnetic. In some embodiments, an antigen is immobilized on the solid surface.

Examples of surfaces include an organic and inorganic polymers, as well as other materials, both natural and synthetic. Specific, non-limiting examples of solid surfaces include nitrocellulose, nylon, glass, fused silica, diazotized membranes (paper or nylon), silicones, cellulose, and cellulose acetate. In addition, plastics such as polyethylene, polypropylene, polystyrene, and the like can be used. Other materials which may be employed include paper, ceramics, metals, metalloids, semiconductive materials, cermets, or the like. In addition, substances that form gels can be used. Such materials include proteins (e.g., gelatins), lipopolysaccharides, silicates, agarose and polyacrylamides. Where the solid surface is porous, various pore sizes may be employed depending upon the nature of the system.

An antigen can be immobilized by an antigen binder to a solid surface. The antigen may be immobilized on the solid surface by an antigen-binding biomolecule attached to the solid surface. The antigen may be immobilized on the solid surface by an oligonucleotide attached to the solid surface. In some embodiments, an antigen may be immobilized on the solid surface by an antigen binder attached to the solid surface.

In some embodiments, components of a reaction are not attached to a solid surface. In these embodiments, the components of a reaction are in a homogenous solution. The components of the reaction may comprise, an oligonucleotide, an antigen, an antibody, an antigen-binder, an antigen-binding biomolecule.

### Detectable Nucleic Acids/Nucleic Acid Products (Compositions and Methods)

Aspects disclosed herein relate to detectable nucleic acids formed from association of a plurality of antigen-binders according to the disclosure, which permit detection of analytes when multiple antigen-binders bind a common antigen. In some embodiments, the nucleic acids are detectable by the formation of a reporter structure from the combination of antigen-binders which may comprise nucleic acids. As a non-limiting example, a first antigen-binder can comprise a first antigen binding moiety and a first nucleic acid and a second antigen-binder can comprise a second antigen binding moiety and a second nucleic acid. In the presence of an antigen, the first antigen binding moiety from the first antigen-binder and the second antigen binding moiety from the second antigen-binder binds to the antigen. The binding of the first and second binding moieties to the antigen place the first and second nucleic acids of the first and second antigen-binders in close proximity and the first and second nucleic acids can bind to one another. The formation of a nucleic acid with the first and second nucleic acids may comprise a unique sequence that allows for detection of the nucleic acid and further may allow for detection of the antigen.

In some embodiments, a nucleic acid of an antigen-binder may comprise a barcode. In some embodiments, a nucleic acid of an antigen-binder may comprise a unique molecular identifier (UMI). In some embodiments, the barcode or UMI may be specific to an antigen binder. In some embodiments, the combination of multiple barcodes or UMIs specific to multiple antigen-binders in a continuous nucleic acid sequence may indicate the presence of an analyte in a sample. In those depicted embodiments, antigen-binders comprising the detected barcode or UMI is specific to an analyte.

In some embodiments, conjugated nucleic acids may form a template for detectable nucleic acids. In some embodiments, the detectable nucleic acids comprise a combination of all nucleic acids from antigen binders specific to an antigen. The detectable nucleic acid may be linear. The detectable nucleic acid may be circular. In some embodiments, the organization of detectable nucleic acids can be used to infer information regarding the detection event. A detectable nucleic acid may comprise a UML. A detectable nucleic acid may comprise more than one UMI. In some embodiments, a combination of UMIs can correspond to a single molecule of an antigen. In those embodiments, the concentration of an analyte may be determined from the combinations of UMIs.

### Detectable Circular Nucleic Acids

Aspects disclosed herein include circular detectable nucleic acids (e.g. circular nucleic acid products resulting from antigen-binding methods according to the disclosure). In some embodiments, the circular nucleic acids are detectable. In some embodiments, the circular nucleic acids comprise nucleic acids from at least two antigen binders. In some embodiments, the circular nucleic acids comprise nucleic acids from two antigen binders. In some embodiments, the circular nucleic acids comprise nucleic acids from three antigen binders. In some embodiments, the circular nucleic acids comprise nucleic acids from four antigen binders. In some embodiments, the circular nucleic acids comprise nucleic acids from more than four antigen binders. In some embodiments, the nucleic acids of the antigen binders are double-stranded. In some embodiments, the nucleic acids of the antigen binders are partially double-stranded. In some embodiments, the nucleic acids of the antigen binders are fully double-stranded.

In some embodiments, the circular detectable nucleic acid is formed when the nucleic acids from antigen binders come in close contact to one another in the presence of a predetermined antigen (e.g. when the antigen binders bind a common molecule of an antigen) via hybridization of their respective toehold or overhang regions. In some embodiments, the production of a circular nucleic acid further comprises incubating a complex of hybridized multiple nucleic acids provided on antigen binders with an enzyme to join at least one strand of the hybridized nucleic acids into a continuous molecule. In some embodiments, the enzyme comprises a ligase (e.g. Ampligase, T4 DNA ligase, T7 DNA ligase, E. coli DNA ligase, HiFi Taq ligase, or Taq DNA ligase).

In some embodiments, the circular detectable nucleic acid is formed by a first partially double-stranded nucleic acid comprises a first proximal nucleic acid linked to a first antigen binder comprising a common hybridization region and an unhybridized overhanging 5′ end and a first distal nucleic acid comprising a common hybridization region and an unhybridized overhanging 5′ end, and a second partially double-stranded nucleic acid comprises a second proximal nucleic acid linked to a second antigen binder comprising a common hybridization region and an unhybridized overhanging 5′ end and a second distal nucleic acid comprising a common hybridization region and an unhybridized overhanging 5′ end. In these depicted embodiments, the first distal nucleic acid is hybridized to the first proximal nucleic acid by the hybridization region, and the second distal nucleic acid is hybridized to the second proximal nucleic acid by the hybridization region. In these depicted embodiments, the first partially double stranded nucleic acid comprises a 5′ overhang on each end and a double-stranded hybridization region, and the second partially double stranded nucleic acid comprises a 5′ overhang on each end and a double-stranded hybridization region. In some embodiments, the free unhybridized 5′ end of the first proximal nucleic acid is configured to bind to the unhybridized overhanging 5′ end of the second distal nucleic acid, and the unhybridized overhanging 5′ end of the second proximal nucleic acid is configured to bind to the unhybridized overhanging 5′ end of the first distal nucleic acid.

In some embodiments, the circular detectable nucleic acid is formed by a first partially double-stranded nucleic acid comprises a first proximal nucleic acid linked to a first antigen binder comprising a common hybridization region and an unhybridized overhanging 3′ end and a first distal nucleic acid comprising a common hybridization region and an unhybridized overhanging 3′ end, and a second partially double-stranded nucleic acid comprises a second proximal nucleic acid linked to a second antigen binder comprising a common hybridization region and an unhybridized overhanging 3′ end and a second distal nucleic acid comprising a common hybridization region and an unhybridized overhanging 3′ end. In these depicted embodiments, the first distal nucleic acid is hybridized to the first proximal nucleic acid by the hybridization region, and the second distal nucleic acid is hybridized to the second proximal nucleic acid by the hybridization region. In these depicted embodiments, the first partially double stranded nucleic acid comprises a 3′ overhang on each end and a double-stranded center of the hybridization region, and the second partially double stranded nucleic acid comprises a 3′ overhang on each end and a double-stranded center of the hybridization region. In some embodiments, the free unhybridized 3′ end of the first proximal nucleic acid is configured to bind to the unhybridized overhanging 3′ end of the second distal nucleic acid, and the unhybridized overhanging 3′ end of the second proximal nucleic acid is configured to bind to the unhybridized overhanging 3′ end of the first distal nucleic acid.

In some embodiments, the circular detectable nucleic acid is formed from a combination of two or more partially double-stranded nucleic acids. In some embodiments, the circular detectable nucleic acid is formed from a combination of a first partially double-stranded nucleic acid and a second partially double-stranded nucleic acid. In some embodiments, the circular nucleic acid is formed from a combination of a first partially double-stranded nucleic acid, a second partially double-stranded nucleic acid, and a third partially double-stranded nucleic acid. In some embodiments, the circular nucleic acid is formed from a combination of a first partially double-stranded nucleic acid, a second partially double-stranded nucleic acid, a third partially double-stranded nucleic acid, and a fourth partially double-stranded nucleic acid. In some embodiments, the circular nucleic acid is formed from a combination of a first partially double-stranded nucleic acid, a second partially double-stranded nucleic acid, a third partially double-stranded nucleic acid, a fourth partially double-stranded nucleic acid, and a fifth partially double-stranded nucleic acid.

In some cases, the circular detectable nucleic acid is formed from a combination of two or more partially double-stranded nucleic acids and a tunable partially double-stranded nucleic acid bridging at least two of the partially double-stranded nucleic acids to form a continuous product. A tunable partially double stranded nucleic acid may not be attached to an antibody. In some embodiments, the circular nucleic acid is formed from a combination of a first partially double-stranded nucleic acid, a second partially double-stranded nucleic acid, and a third partially double-stranded nucleic acid. In these embodiments, the third partially double-stranded nucleic acid may be from a third antigen-binder. In some embodiments, the third partially double-stranded nucleic acid comprises a third proximal nucleic acid linked to the third antigen binding moiety and a third distal nucleic acid hybridized to the third proximal nucleic acid.

In some embodiments, the tunable nucleic acid can comprise a partially double-stranded nucleic acid the concentration of which can be adjusted in methods according to the disclosure to alter sensitivity of the detection method or assay. In some cases where a detection method according to the disclosure utilizes antigen binders comprising a first and second antigen binder each comprising corresponding partially double-stranded nucleic acids, a third tunable nucleic acid can be included in the reaction, which is configured to template production of a linear or circular nucleic acid product (e.g. detectable nucleic acid) from the corresponding partially double-stranded nucleic acids. Without wishing to be bound by theory, by adjusting the concentration of this tunable nucleic acid, the ease of formation of the linear or circular nucleic acid product at a given antigen concentration can be adjusted, with higher concentrations of tunable nucleic acid increasing the propensity to form product and lower concentrations of tunable nucleic acid lowering the propensity to form product. In some embodiments, a tunable nucleic acid comprises a first or 5′ overhanging single-stranded end complementary to an overhanging end of a partially double-stranded nucleic acid attached to said first antigen binder and a second or 3′ overhanging single-stranded end complementary to an overhanging end of a partially double-stranded nucleic acid attached to said second antigen binder.

### Detectable Linear Nucleic Acids/Linear Nucleic Acid Products

Aspects disclosed herein include linear detectable nucleic acids (e.g. linear nucleic acid products resulting from antigen-binding methods according to the disclosure). In some embodiments, the linear nucleic acids are detectable. In some embodiments, the linear nucleic acids comprise nucleic acids from at least two antigen binders. In some embodiments, the linear nucleic acids comprise nucleic acids from two antigen binders. In some embodiments, the linear nucleic acids comprise nucleic acids from three antigen binders. In some embodiments, the linear nucleic acids comprise nucleic acids from four antigen binders. In some embodiments, the linear nucleic acids comprise nucleic acids from more than four antigen binders. In some embodiments, the nucleic acids of the antigen binders are double-stranded. In some embodiments, the nucleic acids of the antigen binders are partially double-stranded.

In some embodiments, the linear detectable nucleic acid is formed when the nucleic acids from antigen binders come in close contact to one another in the presence of a predetermined antigen (e.g. when the antigen binders bind a common molecule of an antigen) via hybridization of their respective toehold or overhang regions. In some embodiments, the production of a linear nucleic acid further comprises incubating a complex of hybridized multiple nucleic acids provided on antigen binders with an enzyme to join at least one strand of the hybridized nucleic acids into a continuous molecule. In some embodiments, the enzyme comprises a ligase (e.g. Ampligase, T4 DNA ligase, T7 DNA ligase, E. coli DNA ligase, HiFi Taq ligase, or Taq DNA ligase).

In some embodiments, the linear detectable nucleic acid is formed by a first partially double-stranded nucleic acid comprises a first proximal nucleic acid linked to a first antigen binder comprising a common hybridization region and an unhybridized overhanging 5′ end and a first distal nucleic acid comprising a common hybridization region and an unhybridized overhanging 5′ end, and a second partially double-stranded nucleic acid comprises a second proximal nucleic acid linked to a second antigen binder comprising a common hybridization region and an unhybridized overhanging 5′ end and a second distal nucleic acid comprising a common hybridization region and an unhybridized overhanging 5′ end. In these depicted embodiments, the first distal nucleic acid is hybridized to the first proximal nucleic acid by the hybridization region, and the second distal nucleic acid is hybridized to the second proximal nucleic acid by the hybridization region. In these depicted embodiments, the first partially double stranded nucleic acid comprises a 5′ overhang on each end and a double-stranded hybridization region, and the second partially double stranded nucleic acid comprises a 5′ overhang on each end and a double-stranded hybridization region. In some embodiments, the free unhybridized 5′ end of the first proximal nucleic acid is configured to bind to the unhybridized overhanging 5′ end of the second distal nucleic acid, and the unhybridized overhanging 5′ end of the second proximal nucleic acid is configured to bind to the unhybridized overhanging 5′ end of the first distal nucleic acid.

In some embodiments, the linear detectable nucleic acid is formed by a first partially double-stranded nucleic acid comprises a first proximal nucleic acid linked to a first antigen binder comprising a common hybridization region and an unhybridized overhanging 3′ end and a first distal nucleic acid comprising a common hybridization region and an unhybridized overhanging 3′ end, and a second partially double-stranded nucleic acid comprises a second proximal nucleic acid linked to a second antigen binder comprising a common hybridization region and an unhybridized overhanging 3′ end and a second distal nucleic acid comprising a common hybridization region and an unhybridized overhanging 3′ end. In these depicted embodiments, the first distal nucleic acid is hybridized to the first proximal nucleic acid by the hybridization region, and the second distal nucleic acid is hybridized to the second proximal nucleic acid by the hybridization region. In these depicted embodiments, the first partially double stranded nucleic acid comprises a 3′ overhang on each end and a double-stranded hybridization region, and the second partially double stranded nucleic acid comprises a 3′ overhang on each end and a double-stranded hybridization region. In some embodiments, the free unhybridized 3′ end of the first proximal nucleic acid is configured to bind to the unhybridized overhanging 3′ end of the second distal nucleic acid, and the unhybridized overhanging 3′ end of the second proximal nucleic acid is configured to bind to the unhybridized overhanging 3′ end of the first distal nucleic acid.

In some embodiments, the linear detectable nucleic acid is formed from a combination of two or more partially double-stranded nucleic acids. In some embodiments, the two or more partially double-stranded nucleic acids are not substantially complementary or configured to hybridize to one another. In some embodiments, the two or more partially double-stranded nucleic acids are configured to template production of a linear nucleic acid product in the presence of at least one single-stranded template oligonucleotide, wherein the single-stranded template oligonucleotide links two of the partially double-stranded oligonucleotides (e.g. via respective overhangs in the two of the partially double-stranded oligonucleotides). In some embodiments, the linear detectable nucleic acid is formed from a combination of a first partially double-stranded nucleic acid and a second partially double-stranded nucleic acid (e.g. with a first template oligonucleotide linking the two, e.g. for ligation). In some embodiments, the linear nucleic acid is formed from a combination of a first partially double-stranded nucleic acid, a second partially double-stranded nucleic acid, and a third partially double-stranded nucleic acid (e.g. with a first template oligonucleotide linking the first and second double-stranded nucleic acid, and a second template oligonucleotide linking the second and third double-stranded nucleic acid, e.g. for ligation). In some embodiments, the linear nucleic acid is formed from a combination of a first partially double-stranded nucleic acid, a second partially double-stranded nucleic acid, a third partially double-stranded nucleic acid, and a fourth partially double-stranded nucleic acid (e.g. with three template oligonucleotides linking the respective double-stranded nucleic acids, e.g. for ligation). In some embodiments, the linear nucleic acid is formed from a combination of a first partially double-stranded nucleic acid, a second partially double-stranded nucleic acid, a third partially double-stranded nucleic acid, a fourth partially double-stranded nucleic acid, and a fifth partially double-stranded nucleic acid (e.g. with four template oligonucleotides linking the respective double-stranded nucleic acids, e.g. for ligation).

In some cases, the linear detectable nucleic acid is formed from a combination of two or more partially double-stranded nucleic acids and a tunable partially double-stranded nucleic acid bridging at least two of the partially double-stranded nucleic acids to form a continuous product. A tunable partially double stranded nucleic acid may not be attached to an antibody. In some embodiments, the linear nucleic acid is formed from a combination of a first partially double-stranded nucleic acid, a second partially double-stranded nucleic acid, and a third partially double-stranded nucleic acid. In these embodiments, the third partially double-stranded nucleic acid may be from a third antigen-binder. In some embodiments, the third partially double-stranded nucleic acid comprises a third proximal nucleic acid linked to the third antigen binding moiety and a third distal nucleic acid hybridized to the third proximal nucleic acid.

### Detection of Linear or Circular Nucleic Acid Product

In some cases, the method may comprise detecting a linear or circular detectable nucleic acid by a specific nucleic acid detection method. In some embodiments, nucleic acids are detected using a nucleic acid-based detection assay (e.g., genotyping array, quantitative polymerase chain reaction (qPCR), whole genome sequencing, skim sequencing, or fluorogenic qPCR). In some embodiments, the nucleic acid-based detection assay comprises qPCR, gel electrophoresis (including for e.g., Northern or Southern blot), immunochemistry, in situ hybridization such as fluorescent in situ hybridization (FISH), cytochemistry, or sequencing. In some embodiments, the sequencing technique comprises next generation sequencing. In some embodiments, the methods involve a hybridization assay such as fluorogenic qPCR (e.g., TaqMan™ or SYBR green).

qPCR

In some cases, the nucleic acid may be detected by “real time amplification” methods also called quantitative PCR (qPCR) or Taqman (see, e.g., U.S. Pat. No. 5,210,015 to Gelfand, U.S. Pat. No. 5,538,848 to Livak, et al., and U.S. Pat. No. 5,863,736 to Haaland, as well as Heid, C. A., et al., Genome Research, 6:986-994 (1996); Gibson, U. E. M, et al., Genome Research 6:995-1001 (1996); Holland, P. M., et al., Proc. Natl. Acad. Sci. USA 88:7276-7280, (1991); and Livak, K. J., et al., PCR Methods and Applications 357-362 (1995)). The basis for this method of monitoring the formation of amplification product is to measure continuously PCR product accumulation using a dual-labeled fluorogenic oligonucleotide probe. The probe used in such assays can be a short (ca. 20-25 bases) polynucleotide that is labeled with two different fluorescent dyes. The 5′ terminus of the probe can be attached to a reporter dye and the 3′ terminus is attached to a quenching dye. The probe is designed to have at least substantial sequence complementarity with a site on the target nucleic acid. Upstream and downstream PCR primers that bind to flanking regions of the locus are also added to the reaction mixture. When the probe is intact, energy transfer between the two fluorophores occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector. The recorded values can then be used to calculate the increase in normalized reporter emission intensity on a continuous basis.

ddPCR

Droplet digital PCR (ddPCR) refers to a digital PCR assay that measures absolute quantities by counting nucleic acid molecules encapsulated in discrete, volumetrically defined, water-in-oil droplet partitions that support PCR amplification (Hinson et al., 2011, Anal. Chem. 83:8604-8610; Pinheiro et al., 2012, Anal. Chem. 84:1003-1011). A single ddPCR reaction may be comprised of at least 20,000 partitioned droplets per well.

A droplet or water-in-oil droplet refers to an individual partition of the droplet digital PCR assay. A droplet supports PCR amplification of template molecule(s) using homogenous assay chemistries and workflows similar to those widely used for real-time PCR applications (Hinson et al., 2011, Anal. Chem. 83:8604-8610; Pinheiro et al., 2012, Anal. Chem. 84:1003-1011).

Droplet digital PCR may be performed using any platform that performs a digital PCR assay that measures absolute quantities by counting nucleic acid molecules encapsulated in discrete, volumetrically defined, water-in-oil droplet partitions that support PCR amplification. The strategy for droplet digital PCR may be summarized as follows: a sample is diluted and partitioned into thousands to millions of separate reaction chambers (water-in-oil droplets) so that each contains one or no copies of the nucleic acid molecule. The number of “positive” droplets detected, which contain the target amplicon (e.g. nucleic acid molecule), versus the number of “negative” droplets, which do not contain the target amplicon (e.g. nucleic acid molecule), may be used to determine the number of copies of the nucleic acid molecule that were in the original sample. Examples of droplet digital PCR systems include the QX100™ Droplet Digital PCR System by Bio-Rad, which partitions samples containing nucleic acid template into 20,000 nanoliter-sized droplets; and the RainDrop™ digital PCR system by RainDance, which partitions samples containing nucleic acid template into 1,000,000 to 10,000,000 picoliter-sized droplets.

### Microarray

Methods for detecting nucleic acids may include array-based methods such as microarray (Schena et al., Science 270:467-70, 1995). By “microarray” is intended an ordered arrangement of hybridizable array elements, such as, for example, polynucleotide probes, on a substrate. The term “probe” refers to any molecule that is capable of selectively binding to a specifically intended target biomolecule, for example, a nucleotide transcript or a protein encoded by or corresponding to an intrinsic gene. Probes can be synthesized by an appropriate procedure, or derived from appropriate biological preparations. Probes may be specifically designed to be labeled. Examples of molecules that can be utilized as probes include, but are not limited to, RNA, DNA, proteins, antibodies, and organic molecules.

In some embodiments, microarrays are used for expression profiling. Each array comprises a reproducible pattern of capture probes attached to a solid support. Labeled RNA or DNA is hybridized to complementary probes on the array and then detected by laser scanning. Hybridization intensities for each probe on the array are determined and converted to a quantitative value representing relative levels. See, for example, U.S. Pat. Nos. 6,040,138, 5,800,992 and 6,020,135, 6,033,860, and 6,344,316. High-density oligonucleotide arrays are particularly useful.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

In a specific embodiment of the microarray technique, PCR amplified inserts are applied to a substrate in a dense array. The microarrayed nucleic acids, immobilized on the microchip, are suitable for hybridization under stringent conditions. Fluorescently labeled probes can be generated through incorporation of fluorescent nucleotides. Labeled probes applied to the chip hybridize with specificity to each spot of nucleic acids on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled probes generated from two sources of nucleic acids are hybridized pairwise to the array. Microarray analysis can be performed by commercially available equipment, following manufacturer's protocols, such as by using the Affymetrix GenChip technology, or Agilent ink jet microarray technology.

### Hybridization-Based Assay

Hybridization-based assays include, but are not limited to, “direct probe” methods such as Southern Blots or In Situ Hybridization (e.g., FISH), and “comparative probe” methods such as Comparative Genomic Hybridization (COH). The methods can be used in a wide variety of formats including, but not limited to substrate (e.g., membrane or glass) bound methods or array-based approaches as described below.

In situ hybridization assays are documented (e.g., Angerer (1987) Meth. Enzymol 152: 649). Generally, in situ hybridization comprises the following major operations: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these operations and the conditions for use vary depending on the particular application.

In an example, in situ hybridization assay, cells can be fixed to a solid support, such as a glass slide. If a nucleic acid is to be probed, the cells can be denatured with heat or alkali. The cells can be then contacted with a hybridization solution at a moderate temperature to permit annealing of labeled probes specific to the nucleic acid sequence encoding the protein. The targets (e.g., cells) can be then washed at a predetermined stringency or at an increasing stringency until an appropriate signal to noise ratio is obtained.

The probes can be labeled, e.g., with radioisotopes or fluorescent reporters. A useful size range is from about 200 bp to about 1000 bases or between about 400 to about 800 bp for double stranded, nick translated nucleic acids.

### Sequencing

The nucleic acid (e.g., linear or circular detectable nucleic acid or nucleic acid product) may be detected by sequencing. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performer by a gene analyzer such as, for example, gene analyzers commercially available from Illumina, Inc., Pacific Biosciences, Inc., or Applied Biosystems/Thermo Fisher Scientific, among many others.

Sequencing methods may include: Next Generation sequencing, high-throughput sequencing, pyrosequencing, classic Sanger sequencing methods, sequencing-by-ligation, sequencing by synthesis, sequencing-by-hybridization, RNA-Seq (Illumina), Digital Gene Expression (Helicos), next generation sequencing, single molecule sequencing by synthesis (SMSS) (Helicos), Ion Torrent Sequencing Machine (Life Technologies/Thermo-Fisher), massively-parallel sequencing, clonal single molecule Array (Solexa), shotgun sequencing, Maxim-Gilbert sequencing, and primer walking. In some embodiments, the sequence comprises whole genome sequencing or skim sequencing. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion Torrent sequencing, PacBio sequencing, and SOLiD sequencing. In some cases, next-generation sequencing involves high-throughput sequencing methods.

### Commercial Kits

Aspects disclosed herein provide a kit for preforming the methods disclosed herein. The kit may comprise one or more of probes, distal oligonucleotides (with indexes), Illumina adapters (such as adapters P5 and P7), magnetic beads, capture antibodies, and instructions. In some embodiments, the kit comprises software for converting *.fastq files into protein counts.

### EXAMPLES

### Example 1. Three Antibody On-Bead Prehybridization

Abs-probes, or probes, were generated by conjugating one of three triple lock oligos (A1, A2, and A3; SEQ ID NOS: 1-3) to native polyclonal antibodies such as anti-IL-1RA polyclonal antibody or anti-GDF-15 polyclonal antibody (Growth Differentiation Factor 15). Oligos A1, A2, and A3 were designed based on secondary structure and cross hybridization and synthesized with a 5′ end amino modification such as 5-Amino-Modifier C12, 5-Amino-Modifier C6, 5-Amino-Modifier C6 dT, and 5-Amino-Modifier UniLink. Each of the three triple lock oligos (A1, A2, and A3) were conjugated to a native polyclonal antibody by modifying the triple lock oligos (A1, A2, and A3) with succinimidyl 4-formylbenzoate (S-4FB) and modifying the antibody with S-HyNic. In brief, the S-4FB was prepared in DMF. The prepared DMF was reacted with the triple lock oligos (A1, A2, and A3). The S-4FB modified triple lock oligos (A1, A2, and A3) were desalted and the buffer exchanged with sodium phosphate at pH 6.0. S-HyNic was prepared by dissolving 2-4 mg of S-HyNic in 100 μL anhydrous DMF or DMSO. The concentration of the antibody was determined and the antibody was brought to a concentration of 1-2.5 mg/mL in 100 mM sodium phosphate and 150 mM sodium chloride at pH 8.0 as needed. The prepared S-HyNic was mixed with the antibody and incubated at room temperature for 2 hours. The S-HyNic modified antibody was desalted and the buffer exchanged with sodium phosphate at pH 6.0.

Each modified triple lock oligo (A1, A2, and A3) was separately incubated with a modified antibody and incubated for 2 hours. Following incubation, the Abs-probes conjugate was purified. The purified Abs-probe conjugates were quantified using a protein assay on a fluorometer.

The Abs-probes (Ab-A1, Ab-A2, and Ab-A3) was diluted to 200 nM in Assay Buffer (1×PBS (phosphate buffer solution), 1 mg/mL BSA (bovine serum albumin), 0.05% Tween 20 (polyoxyethylene₍₂₀₎ sorbitan monolaurate), 15 μg/mL IgG (immunoglobulin G), 0.1 mg/mL salmon sperm DNA, and 5 mM EDTA (Ethylenediaminetetraacetic acid)). Triple lock oligos (O1, O2, and O3) were designed to hybridize with triple lock oligos (A1, A2, and A3) and can have a toehold of t6 (SEQ ID NOS: 7-9) or t9 (SEQ ID NOS: 4-6). Table 1 contains examples sequences used in this example. Each of the triple lock oligos (O1, O2, and O3) were separately diluted to 600 nM in 1×PBSt (phosphate buffer solution with 0.1% tween20).

Each of the probes (Ab-A) were mixed in a 1:1 volume ratio with the corresponding oligo O for an antibody concentration of 100 nM (e.g., probe Ab-A1 with oligo O1). The Ab-A constructs and the O oligos were incubated at room temperature for at least 1 hour. The Ab-A+O pre-hybridizations were diluted to an antibody concentration of 250 pM using assay buffer (probe solution). Fifty μL of probe solution was dispensed into wells containing prepared beads.

The beads were prepared by combining 100 μL of diluted biotinylated polyclonal antibody such as biotinylated anti-IL-1RA diluted to 50 nM in 1×PBS with 500 μg streptavidin beads that had been washed three times in PBSt. The combined biotinylated polyclonal antibody and streptavidin beads were incubated together for 1 hour at room temperature on a rotator before being washed 3 times with 1×PBS with 0.1% tween 20. This generates a stock of Ab-coated beads that can be stored at 4° C. at 5 mg/mL in 1×PBS and 0.5 mg/mL BSA. When preparing for bead capture, the stock Ab-coated beads are vortexed and an aliquot of the mixture is transferred to a new tube. The aliquot of beads are washed in PBSt 1 time and resuspended in assay buffer with 10× original volume of the aliquot resulting in 0.5 mg/mL bead concentration). For example, if a 1 mL aliquot was taken from the resuspended stock of Ab-coated beads the aliquot concentration was approximately 5 mg/mL. After washing, the Ab-coated bead aliquot was resuspended with 10 mL assay buffer bringing the concentration to approximately 0.5 mg/mL.

Following resuspension of the Ab-coated beads to 0.5 mg/mL, the beads were further diluted with assay buffer to a concentration of 0.0185 mg/mL. Forty-five μL of beads were dispensed in each well of a 96-well plate. Five μL of a range of concentrations of target proteins were dispensed in the wells of the 96-well plate for a total of 50 μL in each well. Concentrations of target proteins range from 512 attomolar and 100 nanomolar. The 96-well plate was sealed, vortexed, briefly spun down, and incubated on a rotator at room temperature for 90 minutes. After the incubation, the beads were washed twice in 1×PBSt. All the wash buffer is removed and 50 μL of the probe solution is added to the beads in each well of the 96-well plate. The 96-well plate was sealed, vortexed, briefly spun down, and incubated on a rotator at room temperature for 90 minutes. After the incubation, the beads were washed four times with 1×PBSt. All the wash buffer was removed and 50 μL of ligation master mix (Per sample: 1× Ampligase buffer and 1.25 U Ampligase) was added to each well of the 96-well plate. The 96-well plate was sealed, vortexed, briefly spun down, and incubated at 45° C. for 30 minutes. After the ligation reaction, the plate was washed twice with PBSt.

All the wash buffer was removed, 50 μL of qPCR master mix (1× PowerUp SYBR green master mix (SYBR Green dye, Dual-Lock Taq DNA Polymerase, dNTPs with dUTP/dTTP blend, heat-labile UDG, ROX passive reference dye, and optimized buffer components), 0.5 μM forward primer, and 0.5 μM reverse primer) was added to each well of the 96-well plate. The 96-well plate was sealed, vortexed, and briefly spun down. qPCR was then run on the 96-well plate with the following protocol: 50° C. for 2 minutes, 95° C. for 2 minutes, and sixty-five cycles of 95° C. for 15 seconds, 67° C. for 30 seconds, and 72° C. for 30 seconds.

FIG. 14 depicts an example of the product formed from the protocol described in this example wherein three antibodies are each attached to an oligonucleotide (Ab-A1, Ab-A2, and Ab-A3) and the addition of individual oligonucleotides (O1, O2, and O3) that can hybridize to regions on the oligonucleotides attached to the antibodies can allow for the oligonucleotides attached to the antibodies to become connected and form the circular product described herein. Performing the above protocol using probes with toehold lengths of t6 and t9 at 250 pM concentrations with IL-1RA polyclonal antibody showed the cycle threshold for probes with toehold lengths of nine (t9) to have a lower threshold cycle versus antibody concentration than probes with toehold lengths of nine (t6) (FIG. 2). Performing the above protocol using probes with toehold lengths of t6 and t9 at 250 pM and 25 pM concentrations with IL-1RA polyclonal antibody resulted in similar threshold cycles over antibody concentration for the probe with a toehold of t6 at 250 pM and the probe with a toehold of t9 at 25 pM (FIG. 7A). Additional analysis of the probes with a toehold length t9 at different concentrations yielded similar results as FIG. 7A (FIG. 3). The reproducibility of the protocol above was demonstrated across 10 experiments using probes with a toehold length t9 When the above protocol was repeated with probes with toehold lengths t9 at 250 pM concentrations with IL-1RA polyclonal antibody (FIG. 4 and FIG. 7B). FIG. 5 shows the DNA output for reactions following the same protocol as performed in FIGS. 4 and 7B. In FIG. 5 the amount of antibody loaded on the magnetic beads was 1× for the top line and 10× for the bottom line. A more generic representation of the DNA output against an abundance of target is shown in FIG. 6. In FIG. 6, the protocol used a probe with a toehold t9 at 250 pM as was done in FIGS. 4, 5, and 7B. Performing the above protocol using probes with toehold lengths t9 at varying concentrations with GDF-15 polyclonal antibody resulted in high threshold cycles across probe concentrations (FIG. 9).

### Example 2. Three Antibodies Oligos in Ligation

Abs-probes, or probes, were generated by conjugating one of three triple lock oligos (A1, A2, and A3; SEQ ID NOS: 1-3) to native polyclonal antibodies such as anti-IL-1RA polyclonal antibody or anti-GDF-15 polyclonal antibody (Growth Differentiation Factor 15). Oligos A1, A2, and A3 were designed based on secondary structure and cross hybridization and synthesized with a 5′ end amino modification such as 5-Amino-Modifier C12, 5-Amino-Modifier C6, 5-Amino-Modifier C6 dT, and 5-Amino-Modifier UniLink. Each of the three triple lock oligos (A1, A2, and A3) were conjugated to a native polyclonal antibody by modifying the triple lock oligos (A1, A2, and A3) with succinimidyl 4-formylbenzoate (S-4FB) and modifying the antibody with S-HyNic. In brief, the S-4FB was prepared in DMF. The prepared DMF was reacted with the triple lock oligos (A1, A2, and A3). The S-4FB modified triple lock oligos (A1, A2, and A3) were desalted and the buffer exchanged with sodium phosphate at pH 6.0. S-HyNic was prepared by dissolving 2-4 mg of S-HyNic in 100 μL anhydrous DMF or DMSO. The concentration of the antibody was determined and the antibody was brought to a concentration of 1-2.5 mg/mL in 100 mM sodium phosphate and 150 mM sodium chloride at pH 8.0 as needed. The prepared S-HyNic was mixed with the antibody and incubated at room temperature for 2 hours. The S-HyNic modified antibody was desalted and the buffer exchanged with sodium phosphate at pH 6.0.

Each modified triple lock oligo (A1, A2, and A3) was separately incubated with a modified antibody and incubated for 2 hours. Following incubation, the Abs-probes conjugate was purified. The purified Abs-probe conjugates were quantified using a protein assay on a fluorometer.

The Abs-probes (Ab-A1, Ab-A2, and Ab-A3) was diluted to 200 nM in Assay Buffer (1×PBS (phosphate buffer solution), 1 mg/mL BSA (bovine serum albumin), 0.05% Tween 20 (polyoxyethylene₍₂₀₎ sorbitan monolaurate), 15 μg/mL IgG (immunoglobulin G), 0.1 mg/mL salmon sperm DNA, and 5 mM EDTA (Ethylenediaminetetraacetic acid)).

The Ab-A were diluted to a final antibody concentration of 250 pM using assay buffer (probe solution). In some embodiments, the Ab-A were diluted to other concentrations such as 25 pM. Fifty μL of probe solution was dispensed into wells containing prepared beads. The beads were prepared by combining 100 μL of diluted biotinylated polyclonal antibody such as biotinylated anti-IL-1RA diluted to 50 nM in 1×PBS with 500 μg streptavidin beads that had been washed three times in PBSt. The combined biotinylated polyclonal antibody and streptavidin beads were incubated together for 1 hour at room temperature before being washed 3 times with 1×PBS with 0.1% Tween 20. This generates a stock of Ab-coated beads that can be stored at 4° C. at 5 mg/mL in 1×PBS and 0.5 mg/mL BSA. When preparing for bead capture, the stock Ab-coated beads are vortexed and an aliquot of the mixture is transferred to a new tube. The aliquot of beads are washed in PBSt 1 time and resuspended in assay buffer with 10× original volume of the aliquot resulting in 0.5 mg/mL bead concentration). For example, if a 1 mL aliquot was taken from the resuspended stock of Ab-coated beads the aliquot concentration was approximately 5 mg/mL. After washing, the Ab-coated bead aliquot was resuspended with 10 mL assay buffer bringing the concentration to approximately 0.5 mg/mL.

Following resuspension of the Ab-coated beads to 0.5 mg/mL, the beads were further diluted with assay buffer to a concentration of 0.0185 mg/mL. Forty-five μL of beads were dispensed in each well of a 96-well plate. Five μL of a range of concentrations of target proteins were dispensed in the wells of the 96-well plate for a total of 50 μL in each well. The concentrations of target proteins ranged from 512 attomolar to 100 nanomolar. The 96-well plate was sealed, vortexed, briefly spun down, and incubated on a rotator at room temperature for 90 minutes. After the incubation, the beads were washed twice in 1×PBSt. All the wash buffer is removed and 50 μL of the probe solution is added to the beads in each well of the 96-well plate. The 96-well plate was sealed, vortexed, briefly spun down, and incubated on a rotator at room temperature for 90 minutes. After the incubation, the beads were washed four times with 1×PBSt.

Triple lock oligos (O1, O2, and O3) were designed to hybridize with triple lock oligos (A1, A2, and A3) and can have a toehold of t6 (SEQ ID NOS: 7-9) or t9 (SEQ ID NOS: 4-6). Table 1 contains examples sequences used in this example. Additionally, the triple lock oligos can have additional toehold lengths. Each of the triple lock oligos (O1, O2, and O3) were separately diluted to 600 nM in deionized H₂O. All the wash buffer was removed and 50 μL of ligation master mix (Per sample: 1× Ampligase buffer, 2.5 nM of each triple lock oligo (O1, O2, and O3) and 1.25 U Ampligase) was added to each well of the 96-well plate. The 96-well plate was sealed, vortexed, briefly spun down, and incubated at 45° C. for 30 minutes. After the ligation reaction, the plate was washed twice with PBSt.

All the wash buffer was removed, 50 μL of qPCR master mix (1× PowerUp SYBR green master mix (SYBR Green dye, Dual-Lock Taq DNA Polymerase, dNTPs with dUTP/dTTP blend, heat-labile UDG, ROX passive reference dye, and optimized buffer components), 0.5 μM forward primer, and 0.5 μM reverse primer) was added to each well of the 96-well plate. The 96-well plate was sealed, vortexed, and briefly spun down. qPCR was then run on the 96-well plate with the following protocol: 50° C. for 2 minutes, 95° C. for 2 minutes, and sixty-five cycles of 95° C. for 15 seconds, 67° C. for 30 seconds, and 72° C. for 30 seconds.

FIG. 14 depicts an example of the product formed from the protocol described in this example wherein three antibodies are each attached to an oligonucleotide (Ab-A1, Ab-A2, and Ab-A3) and the addition of individual oligonucleotides (O1, O2, and O3) that can hybridize to regions on the oligonucleotides attached to the antibodies can allow for the oligonucleotides attached to the antibodies to become connected and form the circular product described herein. Performing the above protocol using probes with toehold lengths t12 at varying probe concentrations with IL-1RA polyclonal antibody resulted in high threshold cycles across probe concentrations (FIG. 10). In FIG. 10 the distal nucleic acids, or O1, O2, and O3 were added during the ligation step Performing the above protocol using probes with toehold lengths t9 at 25 pM concentration with GDF-15 polyclonal antibody resulted in high threshold (FIG. 11). The top line in the graph in FIG. 11 corresponds to a reaction protocol wherein 250 pM of O1, O2, and O3 (distal nucleic acids) were added during the ligation step. The bottom line of the graph in FIG. 11 corresponds to a reaction protocol wherein 2.5 nM of O1, O2, and O3 (distal nucleic acids) were added during the ligation step.

TABLE 1
Nucleic acid Sequences Used in this Examples 1 and 2

| | | | SEQ ID |
|---|---|---|---|
| Name | Structure | Sequence | NO: |
| Oligo 1 t9 | Ab1 hybridization | /5Phos/ | SEQ ID |
| | region′-Optional- | CTGTCTCTTATACACATCTGACGCT | NO: 4 |
| | UMII-Index1- | GCCGACGAGAGGTGGAAGTAGGTG | |
| | Toehold2′ | ATGT | |
| | | GATATCGAGGGTGAAGGTGAATAGGG | |
| | | GTCTCGTGGGCTCGGAGATGTGTATAAG | |
| | | AGACAG NNNNNNN GCACAGTT | |
| | | AAAGATCCG | |
| Oligo 2 t9 | Ab2 hybridization | /5Phos/ | SEQ ID |
| | region′-Optional- | AGTAGAGGTGGAGGTAATGTGAAG | NO: 5 |
| | UMI2-Index2- | G NNNNN NNNNNNN TTGCTCCT | |
| | Toehold3′ | CTATTCCCG | |
| Oligo 3 t9 | Ab3 hybridization | /5Phos/ | SEQ ID |
| | region′-Optional- | GGTGGAGGTAAGTTGGAAGTAGAG | NO: 6 |
| | UMI3-Index3- | A NNNNN NNNNNNN AGGTCAAG | |
| | Toehold2′ | TTACGGTCG | |
| Oligo 1 t6 | Ab1 hybridization | /5Phos/ | SEQ ID |
| | region′-Optional- | CTGTCTCTTATACACATCTGACGCT | NO: 7 |
| | UMII-Index1- | GCCGACGAGAGGTGGAAGTAGGTG | |
| | Toehold2′ | ATGT GATATC | |
| | | GAGGGTGAAGGTGAATAGGG | |
| | | GTCTCGTGGGCTCGGAGATGTGTATAAG | |
| | | AGACAG NNNNNNN GCACAGTT | |
| | | GATCCG | |
| Oligo 2 t6 | Ab2 hybridization | /5Phos/ | SEQ ID |
| | | AGTAGAGGTGGAGGTAATGTGAAG | NO: 8 |
| | region′-Optional- | G NNNNN NNNNNNN TTGCTCCT | |
| | UMI2-Index2- | | |
| | Toehold3′ | TTCCCG | |
| Oligo 3 t6 | Ab3 hybridization | /5Phos/ | SEQ ID |
| | region′-Optional- | GGTGGAGGTAAGTTGGAAGTAGAG | NO: 9 |
| | UMI3-Index3- | A NNNNN NNNNNNN AGGTCAAG | |
| | Toehold2′ | TTACGGTCG AGGTCAAG | |
| | | CGGTCG | |
| Ab 1 | Conjugation | /5AmMC12/AA AAA AAA AAA AAA AAA A | SEQ ID |
| | Chemistry-Spacer- | ACATCACCTACTTCCACCTCTCGTCGGC | NO: 1 |
| | Abl hybridization | AGCGTCAGATGTGTATAAGAGACAG | |
| | region-Toehold1 | CGACCGTAA | |
| Ab2 | Conjugation | /5AmMC12/AA AAA AAA AAA AAA AAA A | SEQ ID |
| | Chemistry-Spacer- | CCTTCACATTACCTCCACCTCTACT | |
| | Ab2 hybridization | CGGATCTTT | NO: 2 |
| | region-Toehold2 | | |
| Ab3 | Conjugation | /5AmMC12/AA AAA AAA AAA AAA AAA A | SEQ ID |
| | Chemistry-Spacer- | TCTCTACTTCCAACTTACCTCCACC | NO: 3 |
| | Ab3 hybridization | CGGGAATAG | |
| | region-Toehold3 | | |

Note:
“hybridization regions” can be replaced with a suitable sequencing primer binding sequence.

### Example 3. Circular Product from Two Ab with Tunable Partially Double-Stranded Nucleic Acid

The procedure for detecting an analyte follows Example 1 above, except in this example, two antibodies and two oligos are used. The two antibodies were prehybridized with the corresponding O strands, diluted and then incubated with the beads. Following the 90-minute incubation, the antibodies were washed away in 2×PBSt. The beads were then incubated with a solution comprising the tunable partially double-stranded nucleic acid, or bridge for 15 minutes. During the incubation the bridge hybridizes to the toeholds of the two antibodies present on the beads. Two more washes were performed followed by a ligation reaction. Additionally, the antibodies were specific to Growth Differentiation Factor 15 (GDF-15). Along with the two antibodies and the two oligos, a tunable partially double-stranded nucleic acid is used in the reaction to form the circular product. The tunable partially double-stranded nucleic acid is comprised of two oligos that hybridize to each other and looks like an A oligo hybridized to an O oligo. These two sequences are hybridized together prior to being added to the reaction mixture. The tunable partially double-stranded nucleic acid may be used at a high concentration (100 nM) as in the qPCR graph shown FIG. 8A. The reaction generating the qPCR graph in FIG. 8A also added the tunable partially double-stranded nucleic acid after probe washing. In the reaction generating the qPCR graph in FIG. 8B, the tunable partially double-stranded nucleic acid concentration was 750 pM and the tunable partially double-stranded nucleic acid was added at the same time as the probes rather than washing away excess antibodies. FIG. 15 depicts an example of the product formed for the protocol described and performed to generate the graph in FIG. 8A. In FIG. 15 the reaction contains two antibodies each with attached oligonucleotides. The use of two individual oligonucleotides in combination with a tunable partially double-stranded nucleic acid allows the oligonucleotides attached to the antibodies to generate a circular product through hybridization with the individual oligonucleotides and the tunable partially double-stranded nucleic acid. The results generated using the two antibodies, the two oligos, and the tunable partially double-stranded nucleic acid was consistent across experiments, as shown in FIG. 8C.

### Example 4. Linear Product from Three Ab

The combination of three antibodies and three triple lock probes can be used to generate circular product as above in Example 1 or can follow the same procedure as Example 1 except the combination of the three antibodies and three triple lock probes can also generate a linear product. FIG. 16 depicts an example of the product formed for the protocol described herein. FIG. 16, like FIG. 14 comprises three antibodies each attached to an oligonucleotide and three individual oligonucleotides that can hybridize to the oligonucleotide attached to the antibodies to connect all three antibodies and their attached oligonucleotides. However, in FIG. 16, unlike FIG. 14, the individual oligonucleotides do not allow for the oligonucleotides attached to the antibodies to connect in a fashion that would generate a circular product from the combination of the three antibodies and attached oligonucleotides but rather a linear product. FIG. 12 compares a linear product (such as that depicted in FIG. 16) versus a circular product (such as that depicted in FIG. 14) formed with 25 pM oligo/probes and antibodies specific for IL-1RA.

### Example 5. Linear Product from Two Ab and a Single-Stranded Template Oligonucleotide

The reaction in this example comprises two antibodies attached to partially double-stranded nucleic acids and a single-stranded template oligonucleotide. The single-stranded template oligonucleotide links the partially double-stranded oligonucleotides each attached to an antibody (e.g. via respective overhangs in the two of the partially double-stranded oligonucleotides). FIG. 17 depicts the linear product/intermediate formed when the antibodies and their attached partially double-stranded oligonucleotides are close enough for the single-stranded template oligonucleotide to bind to a portion of each of the two partially double-stranded oligonucleotides attached to an antibody generating a single linear nucleic acid product. The length of the single-stranded template oligonucleotide can be altered depending on the desired distance between the partially double-stranded oligonucleotides attached to an antibody. This procedure can be modified to adjust the length of the single-stranded regions of the partially double-stranded oligos. FIG. 18A shows a qPCR graph when performing this protocol using probes at 250 pM with varying distances with GDF-15 polyclonal antibody. Similar results are shown in FIG. 18B with 52 nucleotides between and different probe concentrations. The recitation of 52 nucleotides between can mean that 26 nucleotides single stranded on one side and 26 nucleotides single stranded on the other side, not including the dsDNA lengths.

### Example 6. Circular Product from Three Ab and Three Single-Stranded Template Oligonucleotides

The reaction in this example comprises three antibodies attached to partially double-stranded nucleic acids and three single-stranded template oligonucleotides. FIG. 19 depicts the product/intermediate of this reaction where each single-stranded template oligonucleotide can connect a portion of the partially double-stranded nucleic acid attached to an antibody with another partially double-stranded nucleic acid attached to an antibody. In example 5 and FIG. 17, the single-stranded template oligonucleotide formed a linear product between two partially double-stranded nucleic acids each attached to antibodies, wherein as described in this example, the additional single-stranded template oligonucleotides continue to connect portions of the partially double-stranded nucleic acids each attached to an antibody until a circular nucleic acid product is generated. FIG. 20 shows a qPCR graph when performing this protocol using probes at varying concentrations with IL-1RA polyclonal antibody.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-72. (canceled)

73. A method for identifying an analyte from a sample, comprising:
(a) providing:
(i) a first analyte binder coupled to said analyte, wherein said first analyte binder is coupled to a first partially double-stranded nucleic acid molecule, wherein a first analyte-binding moiety is coupled to only one strand of said the first partially double-stranded nucleic acid molecule; and
(ii) a first nucleic acid molecule comprising a first index sequence;
(b) subsequent to (a), generating a second nucleic acid molecule with said first partially double-stranded nucleic acid molecule and said first nucleic acid molecule; and
(c) detecting said first index sequence from said second nucleic acid molecule to: (1) detect a presence of said analyte present within said sample, (2) to identify said analyte as originated from said sample, or (3) a combination of (1) and (2).

74. The method of claim 73, further comprising repeating (a)-(c) for one or more additional samples.

75. The method of claim 74, wherein said first index sequence comprises a first sample index sequence.

76. The method of claim 75, wherein (a) further comprises providing a third nucleic acid molecule, and wherein the third nucleic acid molecule comprises a second sample index sequence.

77. The method of claim 76, wherein said sample is identified by a combination of said first sample index sequence and said second sample index sequence.

78. The method of claim 74, wherein a number of unique index sequences detected is less than a number of samples for which (a)-(c) is performed.

79. The method of claim 73, wherein said first index sequence comprises a unique molecular identifier sequence or a sample index sequence.

80. The method of claim 73, wherein said first analyte binder comprises an antigen-binding moiety comprising an antigen-binding fragment, a derivative of an antibody, or a nucleic acid aptamer.

81. The method of claim 73, wherein said first analyte binder comprises an antigen-binding moiety comprising an antibody.

82. The method of claim 73, wherein said sample comprises a bodily fluid, an environmental sample, food, or any combination thereof.

83. The method of claim 82, wherein (c) comprises using sequencing, a quantitative polymerase chain reaction, a microarray, gel electrophoresis, immunochemistry, in situ hybridization, cytochemistry, droplet digital polymerase chain reaction, or any combination thereof to detect said first index sequence from said second nucleic acid molecule.

84. The method of claim 73, where said first partially double-stranded nucleic acid molecule further comprises a primer sequence, a sequencing adapter, an endonuclease site, or any combinations thereof.

85. The method of claim 73, where said analyte comprises a hapten, a metabolite, a sugar, a lipid, a hormone, a phospholipid, or any combination thereof.

86. The method of claim 73, where said analyte comprises a protein.

87. The method of claim 73, wherein (b) comprises: (1) ligating said first partially double-stranded nucleic acid molecule and said first nucleic acid molecule, (2) using a nucleic acid amplification reaction, or (3) a combination of (1)-(2).

88. The method of claim 73, wherein (a) further comprises providing a second analyte binder coupled to said analyte, wherein said second analyte binder is coupled to a second partially double-stranded nucleic acid molecule; and wherein (b) further comprises subsequent to (a), generating said second nucleic acid molecule with said first partially double-stranded nucleic acid molecule, said first nucleic acid molecule, and said second partially double-stranded nucleic acid molecule.

89. A method for identifying an analyte from a sample, comprising:
(a) providing:
(i) a first analyte binder coupled to said analyte, wherein said first analyte binder is coupled to a first partially double-stranded nucleic acid molecule, wherein a single-stranded region of the first partially double-stranded nucleic acid molecule comprises a first index sequence, and
(ii) a second analyte binder coupled to said analyte, wherein said second analyte binder is coupled to a second partially double-stranded nucleic acid molecule, wherein a single-stranded region of the second partially double-stranded nucleic acid molecule comprises a second index sequence;
(b) subsequent to (a), generating a nucleic acid molecule comprising said first index sequence and second index sequence; and
(c) detecting said first index sequence or said second index sequence from said nucleic acid molecule to: (1) detect a presence of said analyte present within said sample, (2) to identify said analyte as originated from said sample, or (3) a combination of (1) and (2).

90. The method of claim 89, wherein said single-stranded region of said first partially double-stranded nucleic acid molecule and said single-stranded region of said second partially double-stranded nucleic acid molecule are configured to hybridize with each other.

91. The method of claim 89, wherein (a) further comprises providing a second nucleic acid molecule comprising a third index sequence; and wherein (b) comprises subsequent to (a), generating said nucleic acid molecule comprising said first index sequence, said second index sequence, and said third index sequence with said first partially double-stranded nucleic acid molecule, said second partially double-stranded nucleic acid molecule, and said second nucleic acid molecule.

92. The method of claim 91, wherein said first index sequence or said second index sequence comprises a unique molecular identifier sequence, and wherein said third index sequence comprises a sample index sequence.

93. The method of claim 91, wherein at least two of said single-stranded region of said first partially double-stranded nucleic acid molecule, said single-stranded region of said second partially double-stranded nucleic acid molecule, or said second nucleic acid molecule are configured to hybridize with one another.

94. The method of claim 91, wherein (1) said single-stranded region of said first partially double-stranded nucleic acid molecule is configured to and hybridize with said single-stranded region of said second partially double-stranded nucleic acid molecule and are not configured to hybridize with each other, and wherein said single-stranded region of said first partially double-stranded nucleic acid molecule and said single-stranded region of said second partially double-stranded nucleic acid molecule are configured to hybridize with said second nucleic acid molecule; (2) said single-stranded region of said second partially double-stranded nucleic acid molecule is configured to hybridize with said single-stranded region of said first partially double-stranded nucleic acid molecule and said second nucleic acid molecule; (3) said second nucleic acid molecule is configured to hybridize with said single-stranded region of said first partially double-stranded nucleic acid molecule and said single-stranded region of said second partially double-stranded nucleic acid molecule; or (4) any combination of (1)-(3).

95. The method of claim 89, wherein said single stranded region of said first nucleic acid molecule or said single-stranded region of said second partially double-stranded nucleic acid molecule is from about 5 nucleotides to about 70 nucleotides long.

96. The method of claim 89, wherein said first index sequence or said second index sequence comprises a unique molecular identifier sequence or a sample index sequence.

97. The method of claim 89, wherein said first analyte binder or said second analyte binder comprises an antigen-binding moiety, and wherein said antigen-binding moiety comprises an antigen-binding fragment, a derivative of an antibody, or a nucleic acid aptamer.

98. The method of claim 89, wherein said first analyte binder or said second analyte binder comprises an antigen-binding moiety comprises an antibody.

99. The method of claim 89, further comprising, prior to (a), contacting said first analyte binder and said second analyte binder to said analyte.

100. The method of claim 89, wherein (c) comprises using sequencing, a quantitative polymerase chain reaction, a microarray, gel electrophoresis, immunochemistry, in situ hybridization, cytochemistry, droplet digital polymerase chain reaction, or any combination thereof to detect said first index sequence and said second index sequence from said nucleic acid molecule.

101. The method of claim 89, where said analyte comprises a hapten, a metabolite, a sugar, a lipid, a hormone, a phospholipid, or any combination thereof.

102. The method of claim 89, where said analyte comprises a protein.
